(19) United States
(12) Patent Application Publication
(10) Pub. No.: US 2025/0388683 A1
(43) Pub. Date: Dec. 25, 2025

(54) ANTI-CANINE INTERLEUKINE-31-RECEPTOR A (IL-31RA) ANTIBODIES AND THE USE THEREOF

(71) Applicant: VETOQUINOL SA, LURE (FR)

(72) Inventor: Olivier LEGER, SAINT-SIXT (FR)

(73) Assignee: VETOQUINOL SA, LURE (FR)

(21) Appl. No.: 18/722,341

(22) PCT Filed: Dec. 20, 2022
(86) PCT No.: PCT/EP2022/087135

§ 371 Date: Jun. 20, 2024

(30) Foreign Application Priority Data
Dec. 20, 2021 (EP) 21306860.4

(51) Int. Cl. C07K 16/28; A61K 39/00; A61P 17/04

(52) U.S. Cl. CPC C07K 16/2866; A61P 17/04; A61K 2039/505; C07K 2317/24; C07K 2317/31; C07K 2317/52; C07K 2317/565; C07K 2317/76

(57) ABSTRACT

The present invention is related to therapeutic anti-canine interleukine-31-receptor A (cIL-31RA) monoclonal antibodies, with high potency regarding inhibition of IL-31RA signaling pathway. The present invention also relates to the use of such antibodies for treating and/or preventing itch and/or inflammatory skin due to atopic dermatitis and allergies in dogs and in particular for treating canine atopic dermatitis.

### REFERENCE TO ELECTRONIC SEQUENCE LISTING

The application contains a Sequence Listing which has been submitted electronically in .XML format and is hereby incorporated by reference in its entirety. Said .XML copy, created on Apr. 4, 2025, is named “3493-0984PUS1.xml” and is 175,774 bytes in size. The sequence listing contained in this .XML file is part of the specification and is hereby incorporated by reference herein in its entirety.

### TECHNICAL FIELD OF THE INVENTION

The present invention is in the field of therapeutic antibodies and especially anti-canine interleukine-31-receptor A (cIL-31RA) monoclonal antibodies, and antigen-binding fragments or antigen-binding derivatives thereof. In particular, the present invention relates to anti-canine IL-31RA monoclonal antibodies of high potency regarding inhibition of IL-31RA signaling pathway. The present invention thus also relates to the use of such antibodies for treating and/or preventing itch and/or inflammatory skin due to atopic dermatitis and allergies in dogs and in particular for treating canine atopic dermatitis.

### BACKGROUND ART

Atopic dermatitis (AD) in dogs is a genetically predisposed chronic inflammatory and pruritic skin disease with characteristic clinical features. Both genetic and environmental factors are involved in the development of the clinical disease, with both types I and IV hypersensitivity reactions demonstrated. Last, a defect in the epidermal barrier is associated to a higher penetration of allergens through the skin and exacerbation of the inflammatory response. Other major exacerbating factors include bacterial (Staphylococcus pseudintermedius) and fungal (Malassezia pachydermatis) infections along with psychogenic and environmental (eg, humidity) factors (Santoro, 2019) (Nuttall et al., 2019). The estimated prevalence of AD in the dog is approximately 10-15% (Gedon and Mueller, 2018).

The age of onset typically spans between 6 months and 6 years; however, more than 70% of AD dogs develop clinical signs between 1 and 3 years of age. The most common clinical signs include generalized pruritus (seasonal, nonseasonal, or nonseasonal with seasonal worsening), erythema, papules, pustules, crusts, and excoriations. Head (perioral, periocular, and ears), flexor aspect of elbows, carpal and tarsal joints, paws (digits, claws, and interdigital aspects), ventral abdomen, perineum, and ventral tail are most commonly affected. Predilection sites differ from breed to breed (Gedon and Mueller, 2018) (Griffin and DeBoer, 2001) (Wilhem S, et al., 2011) (Santoro, 2018).

Clinical, immunological, histological and pathological features of atopic dermatitis in dogs are so similar to the human counterpart, that canine atopic dermatitis has been suggested as an animal model for human AD (Mineshige et al., 2018) (Marsella and Girolomoni, 2009) (Gedon and Mueller, 2018).

The most important limitations of available treatments for canine atopic dermatitis are cost, side effects, compliance and lag phase. Because of their diversity in lag phases and anti-inflammatory/immunomodulatory properties, some therapeutic options are more suitable for treating acute flares (eg, glucocorticoids, oclacitinib), whereas others are more indicated for maintenance and/or prevention of flares (eg, allergen-specific immunotherapy, cyclosporine) (Santoro, 2019).

However, for a medication required for many months/years it is always prudent to find alternative therapies when treatment is needed for extended periods of time (Marsella and De Benedetto, 2017). In addition, long-term use of glucocorticoids is associated with multiple cutaneous and systemic adverse effects because glucocorticoid receptors are present in almost all cells. In addition to some adverse side effects, short-lived benefits of relief are provided by some treatments (oclacitinib), which sometimes are followed by a rapid return of clinical signs even at a higher level than before the initiation of therapy (rebound). Overall, the currently available treatment modalities cannot provide the much-needed convenient, safe, long-term solution, and alternative treatments are needed.

The pathogenesis of atopic dermatitis is however quite complex. It is likely that a defective skin barrier allows microbial adherence, penetration of allergenic proteins, and initiation of abnormal inflammatory and allergic responses. Initially, the immune response in dogs with atopic dermatitis, as in human, is dominated by TH2 cells and involves cytokines such as IL-4, IL-5, IL-6, IL-13, and IL-31 (Marsella, 2012; Olivry et al., 2016), whereas development of chronic inflammation involves a mix of TH1, TH2, TH17, and TH22-cell mediators (Olivry et al., 2016).

Among treatments, the use of monoclonal antibodies was also disclosed. In dogs, a caninized anti-canine IL-31 mAb has been developed to neutralize the effects of canine IL-31 for inducing pruritus in various species, including rodents, dogs, and non-human primates. Despite effectively controlling pruritus in dogs with atopic dermatitis, the anti-canine IL-31 mAb has a limited anti-inflammatory effect on AD skin lesions and inflammation compared to existing therapeutic options like steroids, JAK-inhibitor or cyclosporin (Tamamoto-Mochizuki et al., 2019).

Also, rat antibodies to canine IL31RA able to block the binding of canine IL-31 to canine IL-31RA and use thereof for the treatment of atopic dermatitis in dogs were described for example in provisional applications U.S. 63/092,294 and U.S. 63/092,296 available in the history file of application WO2021/123094 which claims the priority thereof.

### SUMMARY OF THE INVENTION

In the context of the present invention, the inventors surprisingly found new anti-canine interleukine-31-receptor A (cIL-31RA) monoclonal antibodies of particularly high potency regarding inhibition of IL-31RA signaling pathway. Compared to anti-canine IL-31RA monoclonal antibodies of the prior art, such potent antibodies could have the advantage of using lower doses for disease treatment and having a longer lasting effect, thereby allowing subjects to be treated less frequently. This will bring comfort to the subjects to be treated and lower the overall cost of the treatment.

Firstly, the anti-canine IL-31RA monoclonal antibodies of the present invention have a much higher potency than those disclosed for example in the above-mentioned U.S. 63/092,296 (Intervet) since exhibiting a particularly low IC50 for canine IL-31-induced signaling pathway, whereas the lead antibody 28F12 disclosed in the above-mentioned U.S. 63/092,294 (Intervet) shows an IC50 that is at least 5-fold less potent at inhibiting canine IL-31 than the antibodies described in the present invention. Provisional applications U.S. 63/092,294 and U.S. 63/092,296 are available in the history file of application WO2021/123094 which claims the priority thereof.

In a first aspect, the present invention thus relates to an anti-canine interleukine-31-receptor A (cIL-31RA) monoclonal antibody which has the ability to inhibit the signaling pathway activated by the binding of canine IL-31 to canine IL-31RA in a cell-based assay consisting in mammalian cells expressing STAT3, a STAT3-inducible secreted embryonic alkaline phosphatase (SEAP), and canine IL-31RA and OSMRα with an IC50 at least 5 fold lower than a monoclonal anti-cIL-31RA antibody 28F12 comprising a variable region of the heavy chain (VH) consisting of SEQ ID NO: 1 and a variable region of the light chain (VL) consisting of SEQ ID NO: 2.

Among the antibodies of the present invention, it may particularly be mentioned murine 8D3 chimeric antibodies and the corresponding caninized antibodies (including different variants of these caninized antibodies such as 8D3-VHL/8D3-VLH also named VTQ2101, as well as any antibody able to compete with any of these antibodies for binding to IL31RA, and especially able to compete with 8D3-VHL/8D3-VLH antibody.

The present invention also relates to antigen-binding fragments or antigen-binding derivatives of such anti-canine IL-31RA monoclonal antibodies, as well as to a bispecific antibody comprising said antigen-binding fragment or antigen-binding derivative and further comprising another antigen-binding fragment directed to another target relevant for treating atopic dermatitis.

The present invention also relates to a nucleic acid or a combination of two nucleic acids encoding the heavy and/or light chain(s) of the anti-canine IL-31RA monoclonal antibody as described above or of the antigen-binding fragment or antigen-binding derivative thereof, as well as encoding the heavy and/or light chain(s) of the bispecific antibody according to the invention.

The present invention also relates to a vector comprising the nucleic acid(s) according to the invention.

The present invention also relates to a host cell comprising the nucleic acid(s) or vector(s) according to the invention.

The present invention also relates to the anti-canine IL-31RA antibody according to the invention, antigen-binding fragment or antigen-binding derivative thereof, or the bispecific antibody according to the invention, for use as a medicinal product.

The present invention also relates to the anti-canine IL-31RA antibody according to the invention, antigen-binding fragment or antigen-binding derivative thereof, or the bispecific antibody according to the invention, for use in the treatment and/or prevention of itch and/or inflammatory skin due to atopic dermatitis and allergies in dogs, preferably in the treatment of canine atopic dermatitis.

### DESCRIPTION OF THE FIGURES

FIG. 1 represents IL-31 signaling pathway (Nakahara, T., Furue, M. 2018).

FIG. 2 is a summary table of the preferred sequences of the CDRs for one chimeric and several caninized anti-canine IL-31RA antibodies according to the invention and of the resulting consensus sequences of CDRs.

FIG. 3 represents 10% SDS PAGE: Coomassie staining for the purified product in lane 1 under non-reducing (−DTT) and in lane 3 under reducing (+DTT) conditions. 1.3 μg was loaded per lane. In lane 2, molecular weight markers.

FIG. 4 represents titration curves from the HEK Blue STAT3 canine IL-31 SEAP reporter activation for 8D3 (curve with black squares) and 3F1 (curve with grey circles) chimeric canine IgGB antibodies. A negative control with cells only (Cell control, without IL-31 addition and without antibody, which represents the lowest OD reading possible; background of the assay, see single grey triangle) and a positive control with IL-31 addition but without antibody (Interleukin 31 control, which represents the maximum OD reading possible, see single black square) are also presented.

FIG. 5 represents titration curves from the HEK Blue STAT3 canine IL-31 SEAP reporter activation for VTQ2101 (curve with black squares), Tirnovetmab (curve with grey triangles pointing at bottom), Lokivetmab (Cytopoint®, curve with black diamonds), and Nemolizumab (curve with black triangles pointing at top), antibodies. A negative control with cells only (Cell control, without IL-31 addition and without antibody, which represents the lowest OD value; background of the assay, see single grey square) and a positive control with IL-31 addition but without antibody (Interleukin 31 control, which represents the maximum OD value that can be obtained, see single grey circle) are also presented.

FIG. 6 represents titration curves from the HEK Blue STAT3 canine IL-31 SEAP reporter activation for VTQ2101 (curve with black triangles pointing at bottom) and Intervet candidates from US provisional application U.S. 62/092,296 and U.S. 63/092,294 (10A12 (curve with small black squares), 51G4 (curve with dark grey triangles pointing at top), 27A10 (curve with light grey triangles pointing at bottom), 44E2 (curve with dark grey diamonds), 4G7 (curve with light grey diamonds), 28F12 (curve with big black squares) and 53B3 (curve with light grey triangles pointing at top) antibodies, synthesized based on VH/VL sequences disclosed in U.S. 62/092,296 and U.S. 63/092,294). A negative control with cells only (Cell control, without IL-31 addition and without antibody, which represent the lowest OD value possible; background of the assay, see single dark grey circle) and a positive control with IL-31 addition but without antibody (Interleukin 31 control, which represent the maximum OD value that can be obtained, see single big dark grey diamond) are also presented.

FIG. 7 represents titration curves from the HEK Blue STAT3 canine IL-31 SEAP reporter activation for VTQ2101 (curve with light grey diamonds), 3F1 (curve with light grey triangles pointing at bottom) and Intervet candidates from US provisional application U.S. 62/092,296 and U.S. 63/092,294 (28F12 (curve with dark grey triangles pointing at top), 10A12 (curve with black squares) and caninized 10A12 (curve with dark grey diamonds) antibodies, synthesized based on VH/VL sequences disclosed in U.S. 62/092,296 and U.S. 63/092,294). A negative control with cells only (Cell control, without IL-31 addition and without antibody, which represents the lowest OD value that can be obtained, background of the assay, see single grey triangle pointing at top) and a positive control with IL-31 addition but without antibody (Interleukin 31 control, which represents the maximum OD value that can be obtained, see single black square) are also presented.

FIG. 8 represents dermatologic score measured on average (before (baseline) and after IL-31 administration in the three groups of dogs, wherein Group 1 were untreated group, Group 2 received a single subcutaneous injection of Lokivetmab (Cytopoint®) at a dose of 1 mg/kg, and Group 3 received a single subcutaneous injection of 8D3 caninized anti-IL-31RA (VTQ 2101) at 1 mg/kg.

FIG. 9 represents titration curves from the HEK Blue STAT3 canine IL-31 SEAP reporter activation for VTQ2101 (curve with black circles) and scFv-Fc-VTQ2201 (curve with black squares). A negative control with cells only (Cell control, without IL-31 addition and without antibody, which represents the lowest OD reading possible; background of the assay, see single black triangle pointing down) and a positive control with IL-31 addition but without antibody (Interleukin 31 control, which represents the maximum OD reading possible, see single black triangle pointing up) are also presented.

FIG. 10 represents titration curves from the HEK Blue STAT3 canine IL-31 SEAP reporter activation for VTQ2101 (curve with black circles) and scFv-Fc-VTQ2202 (curve with black squares). A negative control with cells only (Cell control, without IL-31 addition and without antibody, which represents the lowest OD reading possible; background of the assay, see single black triangle pointing down) and a positive control with IL-31 addition but without antibody (Interleukin 31 control, which represents the maximum OD reading possible, see single black triangle pointing up) are also presented.

FIG. 11 represents titration curves from the HEK Blue STAT3 canine IL-31 SEAP reporter activation for VTQ2101 (curve with black circles) and scFv-VTQ2101-LH-GS18-Fc (curve with black squares). A negative control with cells only (Cell control, without IL-31 addition and without antibody, which represents the lowest OD reading possible; background of the assay, see single black triangle pointing down) and a positive control with IL-31 addition but without antibody (Interleukin 31 control, which represents the maximum OD reading possible, see single black triangle pointing up) are also presented.

FIG. 12 represents titration curves from the HEK Blue STAT3 canine IL-31 SEAP reporter activation for VTQ2101 (curve with black circles) and scFv-VTQ2101-LHλcap-GS18-Fc (curve with black squares). A negative control with cells only (Cell control, without IL-31 addition and without antibody, which represents the lowest OD reading possible; background of the assay, see single black triangle pointing down) and a positive control with IL-31 addition but without antibody (Interleukin 31 control, which represents the maximum OD reading possible, see single black triangle pointing up) are also presented.

FIG. 13 represents titration curves from the HEK Blue STAT3 canine IL-31 SEAP reporter activation for VTQ2101 (curve with black circles) and scFv-VTQ2102-HLλcap-GS18-Fc (curve with black squares). A negative control with cells only (Cell control, without IL-31 addition and without antibody, which represents the lowest OD reading possible; background of the assay, see single black triangle pointing down) and a positive control with IL-31 addition but without antibody (Interleukin 31 control, which represents the maximum OD reading possible, see single black triangle pointing up) are also presented.

FIG. 14 represents titration curves from the HEK Blue STAT3 canine IL-31 SEAP reporter activation for VTQ2101 (curve with black circles) and scFab-VTQ2101-Fc (curve with black squares). A negative control with cells only (Cell control, without IL-31 addition and without antibody, which represents the lowest OD reading possible; background of the assay, see single black triangle pointing down) and a positive control with IL-31 addition but without antibody (Interleukin 31 control, which represents the maximum OD reading possible, see single black triangle pointing up) are also presented.

### DETAILED DESCRIPTION OF THE INVENTION

### Antibodies

In a first aspect, the present invention relates to an anti-canine interleukine-31-receptor A (cIL-31RA) monoclonal antibody, an antigen-binding fragment or an antigen-binding derivative thereof, which has the ability to inhibit the signaling pathway activated by the binding of canine IL-31 to canine IL-31RA in a cell-based assay consisting in mammalian cells expressing STAT3, a STAT3-inducible secreted embryonic alkaline phosphatase (SEAP), and canine IL-31RA and OSMRα with an IC50 at least 5 fold lower than a monoclonal anti-cIL-31RA antibody 28F12 comprising a variable region of the heavy chain (VH) consisting of SEQ ID NO: 1 and a variable region of the light chain (VL) consisting of SEQ ID NO: 2.

Preferably, the anti-canine interleukine-31-receptor A (cIL-31RA) monoclonal antibody, the antigen-binding fragment or the antigen-binding derivative thereof, according to the present invention has a stronger ability to inhibit the signaling pathway activated by the binding of canine IL-31 to canine IL-31RA as defined above, compared to a monoclonal anti-cIL-31RA antibody 28F12 comprising a heavy chain (HC) consisting of SEQ ID NO: 1 fused to SEQ ID NO: 72 (Canine IgGB WT constant region) and a light chain (LC) consisting of SEQ ID NO: 2 fused to SEQ ID NO: 88 (Canine Kappa type constant region).

Throughout the present description, “canine” may also be referred to as a “dog”. Canines can be categorized as belonging to the subspecies with the trinomial name Canis lupus familiaris (Canis familiaris domesticus) or Canis lupus dingo. Canines include any species of dog Canis sp. and includes both feral and pet varieties, the latter also being referred to as companion animals;

As used herein, “antibody” or “immunoglobulin” means a glycoprotein that specifically binds to another molecule referred to as its “antigen”. An antibody is generally composed of two types of glycopeptide chains called “heavy chain” (abbreviated as “HC”) and “light chain” (abbreviated as “LC”), an antibody being made up of two heavy chains and two light chains, bound by disulfide bridges. Each chain is made up of a variable region and a constant region. The constant region of a particular isotype of heavy or light chain is normally identical from one antibody to another of the same isotype, excluding somatic mutations. In return, the variable region varies from one antibody to another. Indeed, genes coding for antibody heavy chains and light chains are generated by recombination of, respectively, three and two segments of distinct genes called VH, DH and JH-CH for the heavy chain and VL and JL-CL for the light chain. The CH and CL segments do not participate in recombination and form the constant regions of the heavy and light chains respectively. In the constant region, the Fc fragment naturally consists of the heavy chain constant region excluding the CH1 domain and upper hinge region, i.e. the Fc fragment consists of the lower hinge region and the constant domains CH2 and CH3 or CH2 to CH4 (depending on the isotype). Recombinations of the VH-DH-JH and VL-JL segments form the variable regions of heavy and light chains, respectively. The VH and VL regions have three hypervariable zones or complementarity determining regions (CDR) called CDR1, CDR2 and CDR3, the CDR3 region being the most variable, since it is located at the recombination zone. These three CDR regions, and particularly the CDR3 region, are found in the part of the antibody that will be in contact with the antigen and are therefore very important for antigen recognition. Thus, antibodies maintaining the three CDR regions and each of the heavy and light chains of an antibody mostly keep the antigenic specificity of the original antibody. In a certain number of cases, an antibody only maintaining one of the CDRs, and particularly CDR3, also keeps the specificity of the original antibody. The CDR1, CDR2 and CDR3 regions are each preceded by FR1, FR2 and FR3 regions, respectively, corresponding to framework regions (FR) which vary from one VH or VL segment to another. The CDR3 region is also followed by a framework region FR4.

The CDRs of an antibody are defined from the amino acid sequence of its heavy and light chains compared to criteria known to the skilled person. Various methods for determining CDRs have been proposed, and the portion of the amino acid sequence from a heavy or light chain variable region of an antibody defined as a CDR varies depending on the method chosen. The first determination method is the one proposed by Kabat et al. (Kabat et al. Sequences of proteins of immunological interest, 5^{th} Ed., U.S. Department of Health and Human Services, NIH, 1991, and later editions). In this method, CDRs are defined based on sequence variability. Another method was proposed by Chothia et al., 1987. In this method, CDRs are defined based on the location of the structural loop regions. Another method is referred to as “Abm”, which CDRs corresponds to a compromise between the Kabat and Chothia methods (Whitelegg &t Rees, 2000 and 2004). Still another method was proposed by the IMGT, based on determining hypervariable regions. In this method, a unique numbering has been defined to compare variable regions regardless of the antigen receptor, the chain type or the species (Lefranc et al., 2003). This numbering provides a standardized definition of framework regions ((FR1-IMGT: positions 1 to 26, FR2-IMGT: 39 to 55, FR3-IMGT: 66 to 104 and FR4-IMGT: 118 to 128) and complementarity determining regions (CDR1-IMGT: positions 27 to 38, CDR2-IMGT: positions 56 to 65 and CDR3-IMGT: positions 105 to 117). Throughout the present description, the CDR sequences are defined according to the Kabat numbering. In particular, CDRs have been determined by using either IgBLAST, a sequence analysis tool for antibody variable domain sequences, developed by NCBI and freely accessible at https://www.ncbi.nLm.nih.gov/igblast or the program AbNum (antibody numbering) from professor's Andrew C. R. Martin group at UCL website; http://www.bioinf.org.uk/abs/abnum/, which lead to exactly the same CDR sequences.

### Functional Features

In the present invention, the antibody is directed against the canine interleukine-31-receptor A (IL-31RA). cIL-31RA has Gene ID 487212 on Entrez Gene database of NCBI. Three distinct isoforms X1 (787 amino acids, exemplary Reference sequence: XP_038514839.1), X2 (728 amino acids, exemplary Reference sequence: XP_038514842.1) and X3 (649 amino acids, exemplary Reference sequence: XP_038514843.1) of the protein are known.

The amino acid sequence of canine IL-31RA extra cellular domain mature polypeptide chain from isoform X2, which was produced as a recombinant protein and used for immunization and screening is shown below, this sequence has a ten-histidine tag at the C-terminal end; which is as follows (SEQ ID NO: 89):

VLPAKPENISCIFYYEENFTCTWSPEKEASYTWYKVKRTYSYGYKS
DICSTDNSTRGNHASCSFLPPTITNPDNYTIQVEAQNADGIMKSD
ITYWNLDAIMKIEPPEIFSVKSVLGIKRMLQIKWIRPVLAPHSST
LKYTLRFRTINSAYWMEVNFTKEDIDRDETYNLTELQAFTEYVMT
LRCAPAESMFWSGWSQEKVGTTEEEAPYGLDLWRVLKPAMVDGRR
PVQLMWKKATGAPVLEKALGYNIWYFPENNTNLTETVNTTNQTHE
LYLGGKTYWVYVVSYNSLGESPVATLRIPALNEKTFQCIEAMQAC
LTQDQLVVEWQSSAPEVDTWMVEWFPDVDSEPSSFSWESVSQARN
WTIQKDELKPLWCYNISVYPVLRDRVGQPYSTQAYVQEGIPSAGP
VTQADSIGVKTVTITWKEIPKSKRNGFIKNYTIFYQAEDGKEFSK
TVNSNILQYRLESLTRRTSYSLQVMASTNAGGTNGTKINFKTLSI
SVLEGGGGSHHHHHHHHHH

The IL-31RA or interleukine-31-receptor A or interleukine-31-receptor subunit alpha is related to gp130 (IL6ST), the common receptor subunit for IL6-type cytokines. Oncostatin M receptor (OSMR) and IL31RA form the heterodimeric receptor through which IL31 is signaling.

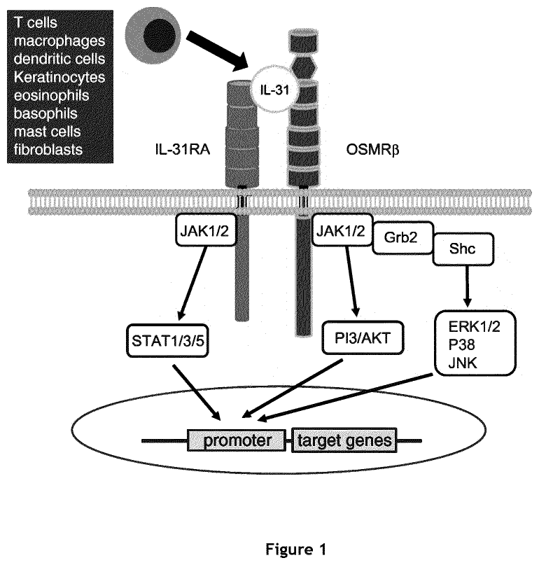

Signaling pathways activated by IL-31 binding to the heterodimeric IL-31RA/OSMR receptor are summarized in FIG. 1.

The antibodies according to the invention bind to canine IL-31RA, and do not bind with significant affinity to canine antigens other than canine IL-31RA. The antibodies according to the invention may however bind to some orthologs of canine IL-31RA. However, the antibodies according to the invention preferably do not bind with significant affinity to human IL-31RA.

The terms “binds” or “binding” as used herein refer to an interaction between molecules to form a complex which, under physiologic conditions, is relatively stable. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions or forces. The strength of the total non-covalent interactions between a single antigen-binding site on an antibody and a single epitope of a target molecule, such as IL-31RA, is the affinity of the antibody or functional fragment for that epitope. The ratio of association (k1) to dissociation (k−1) of an antibody to a monovalent antigen (k1/k−1) is the association constant K, which is a measure of affinity. The value of K varies for different complexes of antibody and antigen and depends on both k1 and k−1. The association constant K for an antibody provided herein can be determined using any method provided herein or any other method well known to those skilled in the art, including Surface Plasmon resonance (SPR) and biolayer interferometry (BLI) technologies. Preferably, antibodies according to the invention bind to canine IL-31RA with an affinity lower than 10E-12 M as measured using BLI technology (Octet K2 instrument). Antibodies according to the invention also preferably do not show any measurable affinity to human IL-31RA by using BLI technology (Octet K2 instrument).

Antibodies according to the invention have the ability to inhibit the signaling pathway activated by the binding of canine IL-31 to canine IL-31RA in a cell-based assay consisting in mammalian cells expressing STAT3, a STAT3-inducible secreted embryonic alkaline phosphatase (SEAP), and canine IL-31RA and OSMRα with an IC50 at least 5 fold lower than a monoclonal anti-cIL-31RA antibody 28F12 comprising a variable region of the heavy chain (VH) consisting of SEQ ID NO: 1 and a variable region of the light chain (VL) consisting of SEQ ID NO: 2.

Signaling pathways activated by IL-31 binding to the heterodimeric IL-31RA/OSMR receptor are summarized in FIG. 1.

“IC50” is defined as the concentration necessary in order to inhibit 50% of a given phenomenon, here preferably the STAT3-signaling. Inhibition of signaling activated by canine IL-31, and in particular of STAT3-signaling activated by canine IL-31, may be measured by any method known in the art. However, especially for measuring the STAT3-signaling activated by canine IL-31, a cell-based assay expressing STAT3, a STAT3-inducible secreted embryonic alkaline phosphatase (SEAP), and canine IL-31RA and OSMRbeta is preferably used.

More preferably, a cell-based assay using mammalian cells expressing, preferably stably, all the necessary signaling pathway components required for evaluation of STAT3-signaling after IL31RA/OSMRbeta heterodimeric receptor activation is used.

Even more preferably, a cell-based assay using HEK293 cells transfected by expression vectors of STAT3, a STAT3-inducible secreted embryonic alkaline phosphatase (SEAP), and canine IL-31RA and OSMRbeta is used for measuring activation of STAT3 transcription factors. Most preferably, the cell-based assay uses the cells deposited under Budapest treaty at Collection Nationale de Cultures de Microorganismes (CNCM), Pasteur Institute, 25 rue du Dr ROUX, 75724 Paris, Cedex 15, under number 1-5792 on Dec. 8, 2021.

No matter the cell-based assay used, it is preferably carried out with culture supernatants or with purified antibodies, and more preferably with purified antibodies.

The cells deposited under Budapest treaty at Collection Nationale de Cultures de Microorganismes (CNCM), Pasteur Institute, 25 rue du Dr ROUX, 75724 Paris, Cedex 15, under number 1-5792 on Dec. 8, 2021, represents a further aspect of the present invention. In addition, another aspect covered by the present invention is a method for screening anti canine IL-31RA antibodies having potent inhibitory ability on IL-31RA signaling pathways using these deposited cells.

Antibodies according to the present invention also encompass an antibody, antigen-binding fragment or antigen-binding derivative which competes for binding to cIL-31RA with a caninized monoclonal anti-cIL-31RA monoclonal antibody comprising:
a) a variable region of the heavy chain (VH) consisting of SEQ ID NO: 3, and
b) a variable region of the light chain (VL) consisting of SEQ ID NO: 4.

The term “compete” when used in the context of antigen binding proteins (e.g. antibodies or antigen-binding fragments or antigen-binding derivatives thereof) that compete for the same epitope means as determined by an assay in which the antigen binding protein being tested prevents or inhibits (e.g., reduces) specific binding of a reference antigen binding protein (e.g., a ligand, or a reference antibody) to a common antigen (e.g., here, cIL-31RA). Numerous types of competitive binding assays can be used to determine if one antigen binding protein competes with another, for example: solid phase direct or indirect radioimmunoassay (RIA), solid phase direct or indirect enzyme immunoassay (EIA), sandwich competition assay (see, e.g., Stahli et al, 1983, Methods in Enzymology 9:242-253); solid phase direct biotin-avidin EIA (see, e.g., Kirkland et al, 1986, J. Immunol. 137:3614-3619) solid phase direct labeled assay, solid phase direct labeled sandwich assay (see, e.g., Harlow and Lane, 1988, Antibodies, A Laboratory Manual, Cold Spring Harbor Press); solid phase direct label RIA using 1-125 label (see, e.g., Morel et al, 1988, Molec. Immunol. 25:7-15); solid phase direct biotin-avidin EIA (see, e.g., Cheung, et al, 1990, Virology 176:546-552); and direct labeled RIA (Moldenhauer et al, 1990, Scand. J. Immunol. 32:77-82). Typically, such an assay involves the use of purified antigen bound to a solid surface or cells bearing the antigen at their surface, an unlabeled test antigen binding protein and a labeled reference antigen binding protein. Competitive inhibition is measured by determining the amount of label bound to the solid surface or cells in the presence of the test antigen-binding protein. Usually, the test antigen binding protein is present in excess. Antigen-binding proteins identified by competition assay (competing antigen binding proteins) include antigen-binding proteins binding to the same epitope as the reference antigen binding protein and antigen binding proteins binding to an adjacent epitope sufficiently proximal to the epitope bound by the reference antigen binding protein for steric hindrance to occur. Usually, when a competing antigen-binding protein is present in excess, it will inhibit (e.g., reduce) specific binding of a reference antigen binding protein to a common antigen by at least 40, at least 45%, at least 50%, at least 55%, at least 60%, at least 65%, at least 70%, at least 75% or more. In some instances, binding is inhibited by at least 80%, at least 85%, at least 90%, at least 95%, at least 97% or more.

Therefore, in the context of the invention, an antibody, antigen-binding fragment or antigen-binding derivative which competes for binding to cIL-31RA with a caninized monoclonal anti-cIL-31RA monoclonal antibody comprising a) a variable region of the heavy chain (VH) consisting of SEQ ID NO: 3, and b) a variable region of the light chain (VL) consisting of SEQ ID NO: 4, preferably reduces specific binding of the caninized monoclonal anti-cIL-31RA monoclonal antibody comprising a) a variable region of the heavy chain (VH) consisting of SEQ ID NO: 3, and b) a variable region of the light chain (VL) consisting of SEQ ID NO: 4 by at least 40, at least 45%, at least 50%, at least 55%, at least 60%, at least 65%, at least 70%, at least 75%, at least 80%, at least 85%, at least 90%, at least 95%, at least 97% or more in an assay measuring the amount of label bound to a solid surface coated with cIL-31RA or cells expressing IL-31RA at their surface in the presence of labeled caninized monoclonal anti-cIL-31RA monoclonal antibody comprising a) a variable region of the heavy chain (VH) consisting of SEQ ID NO: 3, and b) a variable region of the light chain (VL) consisting of SEQ ID NO: 4 and an excess (i.e. a concentration higher than the concentration needed for saturation of all antigen-binding sites on the solid surface or cells) of the competing antibody.

### Structural Features

The percent identities referred to in the context of the disclosure of the present invention are determined on the after optimal global alignment of the sequences to be compared, which may therefore comprise one or more insertions, deletions, truncations and/or substitutions.

This percent identity may be calculated by any sequence analysis method well-known to the person skilled in the art.

The percent identity is determined after global alignment of the sequences to be compared of the sequences taken in their entirety over their entire length. In addition to manual comparison, it is possible to determine global alignment using the algorithm of Needleman and Wunsch (1970).

For nucleotide sequences, the sequence comparison may be performed using any software well-known to a person skilled in the art, such as the Needle software. The parameters used may notably be the following: “Gap open” equal to 10.0, “Gap extend” equal to 0.5, and the EDNAFULL matrix (NCBI EMBOSS Version NUC4.4).

For amino acid sequences, the sequence comparison may be performed using any software well-known to a person skilled in the art, such as the Needle software. The parameters used may notably be the following: “Gap open” equal to 10.0, “Gap extend” equal to 0.5, and the BLOSUM62 matrix.

The percent identify as defined in the context of the present invention is determined via the global alignment of sequences compared over their entire length.

The anti-canine IL-31RA antibodies according to the invention, antigen-binding fragment or antigen-binding derivative thereof, have been shown to have a huge potency for blocking signaling mediated by IL-31.

In particular, despite initial difficulties in obtaining caninized versions maintaining the functions of the chimeric antibodies, several caninized variants of the initial 8D3 antibody exhibit these advantageous properties, and also show ability to improve symptoms associated with IL31 mediated disorders and diseases, especially those associated with atopic dermatitis in dogs.

### Preferred CDRs

In one embodiment, the anti-canine IL-31RA antibody, antigen-binding fragment or antigen-binding derivative according to the invention comprises a heavy chain comprising at least one complementarity determining region (CDR) with the following amino acid sequences to Kabat numbering:
CDR-H-1: X1SFIH (SEQ ID NO: 5), wherein X1 is selected from S or D,
CDR-H-2: X21DPX3X4GX5TEYX6X7X8FQG (SEQ ID NO: 6), wherein:
   X2 is selected from R and Y,
   X3 is selected from A and L,
   X4 is selected from F, N and Q,
   X5 is selected from A, N and G,
   X6 is selected from D and N,
   X7 is selected from A and P,
   X8 is selected from A, N and V,
CDR-H-3: YX9YX10X11SHFDX12 (SEQ ID NO: 7), wherein:
   X9 is selected from H and Y,
   X10 is selected from A and G,
   X11 is selected from A, R, N, T and Q,
   X12 is selected from A and C, and preferably is A.

In another embodiment, the anti-canine IL-31RA antibody, antigen-binding fragment or antigen-binding derivative according to the invention comprises a light chain comprising at least one complementarity determining region (CDR) with the following amino acid sequences to Kabat numbering:
CDR-L-1: KX13SQSVTNDX14T (SEQ ID NO: 8), wherein:
   X13 is selected from S and A,
   X14 is selected from L and V,
CDR-L-2: YASX15RYX16 (SEQ ID NO: 9), wherein:
   X15 is selected from Q, N and I,
   X16 is selected from T, S and P, and
CDR-L-3: QQDYX17SPFT (SEQ ID NO: 10), wherein X17 is selected from A and S.

Advantageously, the anti-canine IL-31RA antibody, antigen-binding fragment or antigen-binding derivative according to the invention comprises:
a) a heavy chain comprising at least one complementarity determining region (CDR) with the following amino acid sequences to Kabat numbering:
CDR-H-1: X1SFIH (SEQ ID NO: 5), wherein X1 is selected from S or D,
CDR-H-2: X21DPX3X4GX5TEYX6X7X8FQG (SEQ ID NO: 6), wherein:
   X2 is selected from R and Y,
   X3 is selected from A and L,
   X4 is selected from F, N and Q,
   X5 is selected from A, N and G,
   X6 is selected from D and N,
   X7 is selected from A and P,
   X8 is selected from A, N and V,
CDR-H-3: YX9YX10X11SHFDX12 (SEQ ID NO: 7), wherein:
   X9 is selected from H and Y,
   X10 is selected from A and G,
   X11 is selected from A, R, N, T and Q,
   X12 is selected from A and C, and preferably is A, and
b) a light chain comprising at least one complementarity determining region (CDR) with the following amino acid sequences to Kabat numbering:
CDR-L-1: KX13SQSVTNDX14T (SEQ ID NO: 8), wherein:
   X13 is selected from S and A,
   X14 is selected from L and V,
CDR-L-2: YASX15RYX16 (SEQ ID NO: 9), wherein:
   X15 is selected from Q, N and I,
   X16 is selected from T, S and P, and
CDR-L-3: QQDYX17SPFT (SEQ ID NO: 10), wherein X17 is selected from A and S.

The anti-canine IL-31RA antibody, antigen-binding fragment or antigen-binding derivative according to the invention may advantageously comprise a heavy chain comprising three CDR-H (heavy chain CDR) with the following amino acid sequences according to Kabat numbering:
CDR-H-1: X1SFIH (SEQ ID NO: 5), wherein X1 is selected from S or D,
CDR-H-2: X21DPX3X4GX5TEYX6X7X8FQG (SEQ ID NO: 6), wherein:
   X2 is selected from R and Y,
   X3 is selected from A and L,
   X4 is selected from F, N and Q,
   X5 is selected from A, N and G,
   X6 is selected from D and N,
   X7 is selected from A and P,
   X8 is selected from A, N and V,
CDR-H-3: YX9YX10X11SHFDX12 (SEQ ID NO: 7), wherein:
   X9 is selected from H and Y,
   X10 is selected from A and G,
   X11 is selected from A, R, N, T and Q,
   X12 is selected from A and C, and preferably is A.

The anti-canine IL-31RA antibody, antigen-binding fragment or antigen-binding derivative according to the invention may also advantageously comprise a light chain comprising three CDR-L (light chain CDR) with the following amino acid sequences according to Kabat numbering:
CDR-L-1: KX13SQSVTNDX14T (SEQ ID NO: 8), wherein:
   X13 is selected from S and A,
   X14 is selected from L and V,
CDR-L-2: YASX15RYX16 (SEQ ID NO: 9), wherein:
   X15 is selected from Q, N and I,
   X16 is selected from T, S and P, and
CDR-L-3: QQDYX17SPFT (SEQ ID NO: 10), wherein X17 is selected from A and S.

Still advantageously, the anti-canine IL-31RA antibody, antigen-binding fragment or antigen-binding derivative according to the invention has heavy and light chains respectively comprising CDR-H and CDR-L with the following amino acid sequences according to Kabat numbering:
CDR-H-1: X1SFIH (SEQ ID NO: 5), wherein X1 is selected from S or D,
CDR-H-2: X21DPX3X4GX5TEYX6X7X8FQG (SEQ ID NO: 6), wherein:
   X2 is selected from R and Y,
   X3 is selected from A and L,
   X4 is selected from F, N and Q,
   X5 is selected from A, N and G,
   X6 is selected from D and N,
   X7 is selected from A and P,
   X8 is selected from A, N and V,
CDR-H-3: YX9YX10X11SHFDX12 (SEQ ID NO: 7), wherein:
   X9 is selected from H and Y,
   X10 is selected from A and G,
   X11 is selected from A, R, N, T and Q,
   X12 is selected from A and C, and preferably is A,
CDR-L-1: KX13SQSVTNDX14T (SEQ ID NO: 8), wherein:
   X13 is selected from S and A,
   X14 is selected from L and V,
CDR-L-2: YASX15RYX16 (SEQ ID NO: 9), wherein:
   X15 is selected from Q, N and G,
   X16 is selected from T, S and P, and
CDR-L-3: QQDYX17SPFT (SEQ ID NO: 10), wherein X17 is selected from A and S.

In all the embodiments described above, X12 is preferably A in CDR-H-3.

Table 1 below summarizes the sequences of the preferred CDRs according to SEQ ID NO: 5 to 10 of the heavy and light chains of anti-canine IL-31RA antibodies, antigen-binding fragment or antigen-binding derivative thereof according to the invention:

| Region | Sequence |
|---|---|
| CDR-H-1 | X1SFIH (SEQ ID NO: 5), |
| | wherein X1 is selected from S or D |
| CDR-H-2 | X2IDPX3X4GX5TEYX6X7X8FQG (SEQ ID NO: 6), wherein: |
| | X2 is selected from R and Y, |
| | X3 is selected from A and L, |
| | X4 is selected from F, N and Q, |
| | X5 is selected from A, N and G, |
| | X6 is selected from D and N, |
| | X7 is selected from A and P, |
| | X8 is selected from A, N and V |
| CDR-H-3 | YX9YX10X11SHFDX12 (SEQ ID NO: 7), wherein: |
| | X9 is selected from H and Y, |
| | X10 is selected from A and G, |
| | X11 is selected from A, R, N, T and Q, |
| | X12 is selected from A and C, and preferably is A, |
| CDR-L-1 | KX13SQSVTNDX14T (SEQ ID NO: 8), wherein: |
| | X13 is selected from S and A, |
| | X14 is selected from L and V |
| CDR-L-2 | YASX15RYX16 (SEQ ID NO: 9), wherein: |
| | X15 is selected from Q, N and I, |
| | X16 is selected from T, S and P |
| CDR-L-3 | QQDYX17SPFT (SEQ ID NO: 10), |
| | wherein X17 is selected from A and S |

### Chimeric and Caninized Antibodies

Any antibody, antigen-binding fragment or antigen-binding derivative according to the invention can advantageously be chimeric or caninized. This prevents canine immune reactions against the antibody administered.

In particular, the antibody according to the invention can advantageously be any one of the chimeric or caninized versions of antibodies 8D3. Antibodies according to the invention thus include mouse monoclonal antibody 8D3 (as well as antigen-binding fragments or antigen-binding derivatives thereof as defined herein).

A “chimeric” antibody means an antibody that contains natural variable regions (light chain and heavy chain) derived from an antibody from a given species in combination with the constant regions of the light chain and heavy chain of an antibody of a species heterologous to said given species (U.S. Pat. No. 4,816,567; and Morrison et al., 1984). Chimeric antibodies according to the invention use non-canine variable regions fused to canine constant regions and can be prepared by using genetic recombinant techniques. For example, chimeric antibodies can be made by cloning recombinant DNA bearing a promoter and a sequence coding for the variable region of a non-canine monoclonal antibody according to the invention and a sequence coding for the constant region of a canine antibody. A chimeric antibody of the invention encoded by such recombinant gene will be, for example, a chicken-canine chimera or a rabbit-canine chimera, the specificity of this antibody being determined by the variable region derived from chicken or rabbit DNA and its isotype determined by the constant region derived from canine DNA. This will notably be the case of chimeric antibodies obtained from mouse monoclonal antibody 8D3 described in the present application, the heavy and light chains of which will be the fusion of a mouse variable region to a canine constant region. For chimeric antibody preparation methods, refer, for example, to Bergeron et al., 2014.

A “caninized” antibody means an antibody that contains CDRs derived from an antibody of non-canine origin, the other parts of the antibody molecule being derived from one (or more) canine antibodies. Caninized antibodies may be prepared using a similar approach as the well-known techniques described for humanization and offer the advantage of reduced immunogenicity when administered as therapeutics to dogs. Procedures for the production of humanized monoclonal antibodies include those described in Riechmann et al., 1988, Liu et al., 1987, Larrick et al., 1989, and Winter and Harris, 1993.

Caninized antibodies according to the invention may be prepared from techniques known to the skilled person. Antibodies were caninized by grafting the three CDRs, as defined by the Kabat nomenclature, from the light chain variable region (VL) into a canine germline VL with a sequence as-homologous-as-possible to the one of the parental antibody VL. Similarly, the three CDRs from the heavy chain variable region (VH) were grafted into a canine germline VH with a sequence as-homologous-as-possible to the parental antibody VH. As used herein, “germline sequence” refers to a sequence of unrearranged immunoglobulin DNA sequences. The source of unrearranged immunoglobulin sequences used for the invention is the IMGT database (Giudicelli et al. Nucl. Acids Res., 2005; http://www.imgt.org). In addition, a few amino acid residues in the canine framework regions of the selected canine germline variable regions may be changed to the amino acid residues that were present in the parental variable regions (so called back-mutations intended to maintain high affinity to the antigen). As used herein the term “canine framework” refers to the amino acid sequence of the heavy chain and light chain of a canine antibody other than the CDR residues as defined by the Kabat nomenclature. Based upon information on the structure of immunoglobulin variable regions, and with the guidance of an homology molecular model of the Fv of the parental monoclonal antibody, a few residues in the framework regions that are identified as having key roles in either maintaining the CDRs in the right conformation or in VH/VL packing, may or not be retained in caninized versions after comparing caninized versions retaining them with caninized versions substituting them with their canine germline counterparts. Under guidance of the homology molecular model, some CDR residues, as defined by KABAT, may also be substituted or not for their canine germline counterparts (so called germlining) in caninized versions when judged possible the CDR residues, in order to increase the degree of canineness (i.e. percentage sequence identity for both VH and VL between the caninized versions and the closest canine germline used as acceptor sequence for the CDR-grafting).

The added-value of combining a structural model with pure sequence analysis is the potential to discriminate between paratope-facing and non-paratopic residues in the CDR regions. The purpose of the structural model is to permit expanding the limits of the caninization process, taking it beyond mere CDR-grafting. Also, the structural models permit making more intelligent choices regarding back-mutations in light of the particular germlines involved. Note that the Kabat CDR definitions are not as strictly structural as those of other systems; thus, for some germlines the Kabat definitions are too broad. For both chains, heavy and light, we can usually be fairly confident that the assignment of residues from CDR1 and 2 as paratopic and non-paratopic, based upon the structural model, is correct. Similarly, the light chain CDR3 is usually well-described with high probability. The difficult case is invariably CDR3 of the heavy chain.

### Chimeric Antibodies

In one embodiment of the invention, the anti-canine IL-31RA antibody, antigen-binding fragment or antigen-binding derivative according to the invention is chimeric.

### CDRs

In one embodiment, the anti-canine IL-31RA antibody, antigen-binding fragment or antigen-binding derivative according to the invention is a chimeric antibody comprising a heavy chain comprising at least one complementarity determining region (CDR) with the following amino acid sequences to Kabat numbering:

CDR-H-1:
(SEQ ID NO: 11)
DSFIH,
CDR-H-2:
(SEQ ID NO: 12)
RIDPANGNTEYDPNFQG,
CDR-H-3:
(SEQ ID NO: 13)
YYYGNSHFDC
or preferably
(SEQ ID NO: 93)
YYYGNSHFDA.

In another embodiment, the anti-canine IL-31RA antibody, antigen-binding fragment or antigen-binding derivative according to the invention is a chimeric antibody comprising a light chain comprising at least one complementarity determining region (CDR) with the following amino acid sequences to Kabat numbering:

CDR-L-1:
(SEQ ID NO: 14)
KASQSVTNDVT,
CDR-L-2:
(SEQ ID NO: 15)
YASNRYT,
CDR-L-3:
(SEQ ID NO: 16)
QQDYSSPFT.

Advantageously, the anti-canine IL-31RA chimeric antibody, antigen-binding fragment or antigen-binding derivative according to the invention has:
a) a heavy chain comprising at least one complementarity determining region (CDR) with the following amino acid sequences to Kabat numbering:

CDR-H-1:
(SEQ ID NO: 11)
DSFIH,
CDR-H-2:
(SEQ ID NO: 12)
RIDPANGNTEYDPNFQG,
CDR-H-3:
(SEQ ID NO: 13)
YYYGNSHFDC
or preferably
(SEQ ID NO: 93)
YYYGNSHFDA,

and
b) a light chain comprising at least one complementarity determining region (CDR) with the following amino acid sequences to Kabat numbering:

CDR-L-1:
(SEQ ID NO: 14)
KASQSVTNDVT,
CDR-L-2:
(SEQ ID NO: 15)
YASNRYT,
CDR-L-3:
(SEQ ID NO: 16)
QQDYSSPFT.

The anti-canine IL-31RA chimeric antibody, antigen-binding fragment or antigen-binding derivative according to the invention may advantageously have a heavy chain comprising three CDR-H (heavy chain CDR) with the following amino acid sequences according to Kabat numbering:

CDR-H-1:
(SEQ ID NO: 11)
DSFIH,
CDR-H-2:
(SEQ ID NO: 12)
RIDPANGNTEYDPNFQG,
and
CDR-H-3:
(SEQ ID NO: 13)
YYYGNSHFDC
or preferably
(SEQ ID NO: 93)
YYYGNSHFDA.

The anti-canine IL-31RA chimeric antibody, antigen-binding fragment or antigen-binding derivative according to the invention may also advantageously have a light chain comprising three CDR-L (light chain CDR) with the following amino acid sequences according to Kabat numbering:

CDR-L-1:
(SEQ ID NO: 14)
KASQSVTNDVT,
CDR-L-2:
(SEQ ID NO: 15)
YASNRYT,
and
CDR-L-3:
(SEQ ID NO: 16)
QQDYSSPFT.

Still advantageously, the anti-canine IL-31RA chimeric antibody, antigen-binding fragment or antigen-binding derivative according to the invention has heavy and light chains respectively comprising CDR-H and CDR-L with the following amino acid sequences according to Kabat numbering:

CDR-H-1:
(SEQ ID NO: 11)
DSFIH,
CDR-H-2:
(SEQ ID NO: 12)
RIDPANGNTEYDPNFQG,
CDR-H-3:
(SEQ ID NO: 13)
YYYGNSHFDC
or preferably
(SEQ ID NO: 93)
YYYGNSHFDA,
CDR-L-1:
(SEQ ID NO: 14)
KASQSVTNDVT,
CDR-L-2:
(SEQ ID NO: 15)
YASNRYT,
and
CDR-L-3:
(SEQ ID NO: 16)
QQDYSSPFT.

In all the embodiments described above, CDR-H-3 is preferably YYYGNSHFDA (SEQ ID NO: 93).

### Variable Regions

In one advantageous embodiment of the invention, the anti-canine IL-31RA antibody, antigen-binding fragment or antigen-binding derivative according to the invention comprises a heavy chain comprising a variable region with at least 80%, at least 85%, at least 90%, at least 95%, at least 96%, at least 97%, at least 98%, or at least 99% identity with SEQ ID NO: 17, preferably a variable region of SEQ ID NO: 17, or a variable region with at least 80%, at least 85%, at least 90%, at least 95%, at least 96%, at least 97%, at least 98%, or at least 99% identity with SEQ ID NO: 94, preferably a variable region of SEQ ID NO: 94.

In another advantageous embodiment of the invention, the anti-canine IL-31RA antibody, antigen-binding fragment or antigen-binding derivative according to the invention comprise a light chain comprising a variable region with at least 80%, at least 85%, at least 90%, at least 95%, at least 96%, at least 97%, at least 98%, or at least 99% identity with SEQ ID NO: 18, preferably a variable region of SEQ ID NO: 18.

Advantageously, the anti-canine IL-31RA antibody, antigen-binding fragment or antigen-binding derivative according to the invention comprises:
a) a variable region of the heavy chain (VH) with at least 80%, at least 85%, at least 90%, at least 95%, at least 96%, at least 97%, at least 98%, or at least 99% identity with SEQ ID NO: 17, preferably a variable region of SEQ ID NO: 17, or a variable region with at least 80%, at least 85%, at least 90%, at least 95%, at least 96%, at least 97%, at least 98%, or at least 99% identity with SEQ ID NO: 94, preferably a variable region of SEQ ID NO: 94, and
b) a variable region of the light chain (VL) with at least 80%, at least 85%, at least 90%, at least 95%, at least 96%, at least 97%, at least 98%, or at least 99% identity with SEQ ID NO: 18, preferably a variable region of SEQ ID NO: 18.

Preferably, the anti-canine IL-31RA antibody, antigen-binding fragment or antigen-binding derivative according to the invention comprises:
1) a variable region of the heavy chain (VH) with at least 80%, at least 85%, at least 90%, at least 95%, at least 96%, at least 97%, at least 98%, or at least 99% identity with SEQ ID NO: 17, or a variable region with at least 80%, at least 85%, at least 90%, at least 95%, at least 96%, at least 97%, at least 98%, or at least 99% identity with SEQ ID NO: 94, preferably a variable region of SEQ ID NO: 94 and a variable region of the light chain (VL) with at least 80%, at least 85%, at least 90%, at least 95%, at least 96%, at least 97%, at least 98%, or at least 99% identity with SEQ ID NO: 18, and
2) wherein said heavy and light chains respectively comprise CDR-H and CDR-L with the following amino acid sequences:
   a heavy chain comprising three CDR-H (heavy chain CDR) with the following amino acid sequences according to Kabat numbering:

CDR-H-1:
(SEQ ID NO: 11)
DSFIH,
CDR-H-2:
(SEQ ID NO: 12)
RIDPANGNTEYDPNFQG,
and
CDR-H-3:
(SEQ ID NO: 13)
YYYGNSHFDC
or preferably
(SEQ ID NO: 93)
YYYGNSHFDA,

and
   a light chain comprising three CDR-L (light chain CDR) with the following amino acid sequences according to Kabat numbering:

CDR-L-1:
(SEQ ID NO: 14)
KASQSVTNDVT,
CDR-L-2:
(SEQ ID NO: 15)
YASNRYT,
and
CDR-L-3:
(SEQ ID NO: 16)
QQDYSSPFT

In particular, the anti-canine IL-31RA antibody, antigen-binding fragment or antigen-binding derivative according to the invention preferably comprises:
2) a variable region of the heavy chain (VH) with at least 80%, at least 85%, at least 90%, at least 95%, at least 96%, at least 97%, at least 98%, or at least 99% identity with SEQ ID NO: 94, preferably a variable region of SEQ ID NO: 94 and a variable region of the light chain (VL) with at least 80%, at least 85%, at least 90%, at least 95%, at least 96%, at least 97%, at least 98%, or at least 99% identity with SEQ ID NO: 18, and
2) wherein said heavy and light chains respectively comprise CDR-H and CDR-L with the following amino acid sequences:
   a heavy chain comprising three CDR-H (heavy chain CDR) with the following amino acid sequences according to Kabat numbering:

CDR-H-3:
(SEQ ID NO: 93)
YYYGNSHFDA,
CDR-H-1:
(SEQ ID NO: 11)
DSFIH,
CDR-H-2:
(SEQ ID NO: 12)
RIDPANGNTEYDPNFQG,
and

and
   a light chain comprising three CDR-L (light chain CDR) with the following amino acid sequences according to Kabat numbering:

CDR-L-1:
(SEQ ID NO: 14)
KASQSVTNDVT,
CDR-L-2:
(SEQ ID NO: 15)
YASNRYT,
and
CDR-L-3:
(SEQ ID NO: 16)
QQDYSSPFT

In one advantageous embodiment of the invention, the anti-canine IL-31RA antibody, antigen-binding fragment or antigen-binding derivative according to the invention comprises a heavy chain comprising a variable region with SEQ ID NO: 17. In another advantageous embodiment of the invention, the anti-canine IL-31RA antibody, antigen-binding fragment or antigen-binding derivative according to the invention comprises a heavy chain comprising a variable region with SEQ ID NO: 94.

In another advantageous embodiment of the invention, the anti-canine IL-31RA antibody, antigen-binding fragment or antigen-binding derivative according to the invention comprises a light chain comprising a variable region with SEQ ID NO: 18.

Advantageously, the anti-canine IL-31RA antibody, antigen-binding fragment or antigen-binding derivative according to the invention comprises a heavy chain comprising a variable region with SEQ ID NO: 17, or a variable region with at least 80%, at least 85%, at least 90%, at least 95%, at least 96%, at least 97%, at least 98%, or at least 99% identity with SEQ ID NO: 94, preferably a variable region of SEQ ID NO: 94 and a light chain comprising a variable region with SEQ ID NO: 18.

More advantageously, the anti-canine IL-31RA antibody, antigen-binding fragment or antigen-binding derivative according to the invention comprises a heavy chain comprising a variable region with SEQ ID NO: 94, and a light chain comprising a variable region with SEQ ID NO: 18.

Table 2 below summarizes the preferred sequences of the variable regions of the heavy and light chains of chimeric anti-canine IL-31RA antibodies and the corresponding CDRs according to the invention:

| | | Corresponding CDRs (Kabat |
|---|---|---|
| Region name | Sequence | numbering) |
| VH chimeric | EVQLQQSGAELVKPGASVKLSCTAS | CDR-H-1: DSFIH (SEQ ID NO: 11), |
| | GFNIKDSFIHWLKQRPEQGLEWIGR | CDR-H-2: RIDPANGNTEYDPNFQG (SEQ |
| | IDPANGNTEYDPNFQGKDTITADTS | ID NO: 12), |
| | SNTAYLQVSSLTSEDTAVYYCARYY | CDR-H-3: YYYGNSHFDC (SEQ ID NO: |
| | YGNSHFDCWGQGTTLTVSS (SEQ | 13) |
| | ID NO: 17) | |
| Preferred VH | EVQLQQSGAELVKPGASVKLSCTAS | CDR-H-1: DSFIH (SEQ ID NO: 11), |
| chimeric | GFNIKDSFIHWLKQRPEQGLEWIGR | CDR-H-2: RIDPANGNTEYDPNFQG (SEQ |
| | IDPANGNTEYDPNFQGKVTITADTS | ID NO: 12), |
| | SNTAYLQLSSLTSEDTAVYYCARYY | CDR-H-3: YYYGNSHFDA (SEQ ID NO: |
| | YGNSHFDAWGQGTTLTVSS (SEQ | 93) |
| | ID NO: 94) | |
| VL chimeric | RIVMNQTPKFLPISAGDRVIITCKAS | CDR-L-1: KASQSVTNDVT (SEQ ID NO: |
| | QSVTNDVTWYQQKPGQSPKVLIHY | 14), |
| | ASNRYTGVPDRFTGSGYGTDFTFTI | CDR-L-2: YASNRYT (SEQ ID NO: 15), |
| | STVQAEDLAVYFCQQDYSSPFTFGS | and |
| | GTKLEIK (SEQ ID NO: 18) | CDR-L-3: QQDYSSPFT (SEQ ID NO: 16) |

### Caninized Antibodies

In another embodiment of the invention, the anti-canine IL-31RA antibody, antigen-binding fragment or antigen-binding derivative according to the invention is caninized.

In another embodiment of the invention, the anti-canine IL-31RA antibody, antigen-binding fragment or antigen-binding derivative according to the invention is caninized and comprises a heavy chain comprising CDR-H-1 of sequence SEQ ID NO: 19, CDR-H-2 of sequence SEQ ID NO: 6, CDR-H-3 of sequence SEQ ID NO: 7 and a light chain comprising CDR-L-1 of sequence SEQ ID NO: 8, CDR-L-2 of sequence SEQ ID NO: 9, CDR-L-3 of sequence SEQ ID NO: 10.

### CDRs

In one embodiment, the anti-canine IL-31RA antibody, antigen-binding fragment or antigen-binding derivative according to the invention is a caninized antibody comprising a heavy chain comprising at least one complementarity determining region (CDR) with the following amino acid sequences to Kabat numbering:
CDR-H-1: X1SFIH (SEQ ID NO: 5), wherein X1 is selected from S or D, and preferably SSFIH (SEQ ID NO: 19),
CDR-H-2: X21DPX3X4GX5TEYX6X7X8FQG (SEQ ID NO: 20), wherein:
   X2 is selected from R and Y,
   X3 is selected from A and L,
   X4 is selected from F and Q,
   X5 is selected from A, N and G,
   X6 is selected from D and N,
   X7 is selected from A and P,
   X8 is selected from A, N and V,
CDR-H-3: YX9YX10X11SHFDA (SEQ ID NO: 21), wherein:
   X9 is selected from H and Y,
   X10 is selected from A and G,
   X11 is selected from A, R, T and Q.

In another embodiment, the anti-canine IL-31RA antibody, antigen-binding fragment or antigen-binding derivative according to the invention is a caninized antibody comprising a light chain comprising at least one complementarity determining region (CDR) with the following amino acid sequences to Kabat numbering:
CDR-L-1: KX13SQSVTNDX14T (SEQ ID NO: 8), wherein:
   X13 is selected from S and A,
   X14 is selected from L and V,
CDR-L-2: YASX15RYX16 (SEQ ID NO: 9), wherein:
   X15 is selected from Q, N and I,
   X16 is selected from T, S and P, and
CDR-L-3: QQDYX17SPFT (SEQ ID NO: 10), wherein X17 is selected from A and S.

Advantageously, the anti-canine IL-31RA caninized antibody, antigen-binding fragment or antigen-binding derivative according to the invention has:
a) a heavy chain comprising at least one complementarity determining region (CDR) with the following amino acid sequences to Kabat numbering:
CDR-H-1: X1SFIH (SEQ ID NO: 5), wherein X1 is selected from S or D, and preferably SSFIH (SEQ ID NO: 19),
CDR-H-2: X21DPX3X4GX5TEYX6X7X8FQG (SEQ ID NO: 20), wherein:
   X2 is selected from R and Y,
   X3 is selected from A and L,
   X4 is selected from F and Q,
   X5 is selected from A, N and G,
   X6 is selected from D and N,
   X7 is selected from A and P,
   X8 is selected from A, N and V,
CDR-H-3: YX9YX10X11SHFDA (SEQ ID NO: 21), wherein:
   X9 is selected from H and Y,
   X10 is selected from A and G,
   X11 is selected from A, R, T and Q.
b) a light chain comprising at least one complementarity determining region (CDR) with the following amino acid sequences to Kabat numbering:
CDR-L-1: KX13SQSVTNDX14T (SEQ ID NO: 8), wherein:
   X13 is selected from S and A,
   X14 is selected from L and V,
CDR-L-2: YASX15RYX16 (SEQ ID NO: 9), wherein:
   X15 is selected from Q, N and I,
   X16 is selected from T, S and P, and
CDR-L-3: QQDYX17SPFT (SEQ ID NO: 10), wherein X17 is selected from A and S.

The caninized anti-canine IL-31RA antibody, antigen-binding fragment or antigen-binding derivative according to the invention may advantageously have a heavy chain comprising three CDR-H (heavy chain CDR) with the following amino acid sequences according to Kabat numbering:
CDR-H-1: X1SFIH (SEQ ID NO: 5), wherein X1 is selected from S or D, and preferably SSFIH (SEQ ID NO: 19),
CDR-H-2: X21DPX3X4GX5TEYX6X7X8FQG (SEQ ID NO: 20), wherein:
   X2 is selected from R and Y,
   X3 is selected from A and L,
   X4 is selected from F and Q,
   X5 is selected from A, N and G,
   X6 is selected from D and N,
   X7 is selected from A and P,
   X8 is selected from A, N and V, and
CDR-H-3: YX9YX10X11SHFDA (SEQ ID NO: 21), wherein:
   X9 is selected from H and Y,
   X10 is selected from A and G,
   X11 is selected from A, R, T and Q.

The caninized anti-canine IL-31RA antibody, antigen-binding fragment or antigen-binding derivative according to the invention may also advantageously have a light chain comprising three CDR-L (light chain CDR) with the following amino acid sequences according to Kabat numbering:
CDR-L-1: KX13SQSVTNDX14T (SEQ ID NO: 8), wherein:
   X13 is selected from S and A,
   X14 is selected from L and V,
CDR-L-2: YASX15RYX16 (SEQ ID NO: 9), wherein:
   X15 is selected from Q, N and I,
   X16 is selected from T, S and P, and
CDR-L-3: QQDYX17SPFT (SEQ ID NO: 10), wherein X17 is selected from A and S.

Still advantageously, the caninized anti-canine IL-31RA antibody, antigen-binding fragment or antigen-binding derivative according to the invention has heavy and light chains respectively comprising CDR-H and CDR-L with the following amino acid sequences according to Kabat numbering:
CDR-H-1: X1SFIH (SEQ ID NO: 5), wherein X1 is selected from S or D, and preferably SSFIH (SEQ ID NO: 19),
CDR-H-2: X21DPX3X4GX5TEYX6X7X8FQG (SEQ ID NO: 20), wherein:
   X2 is selected from R and Y,
   X3 is selected from A and L,
   X4 is selected from F and Q,
   X5 is selected from A, N and G,
   X6 is selected from D and N,
   X7 is selected from A and P,
   X8 is selected from A, N and V,
CDR-H-3: YX9YX10X11SHFDA (SEQ ID NO: 21), wherein:
   X9 is selected from H and Y,
   X10 is selected from A and G,
   X11 is selected from A, R, T and Q.
CDR-L-1: KX13SQSVTNDX14T (SEQ ID NO: 8), wherein:
   X13 is selected from S and A,
   X14 is selected from L and V,
CDR-L-2: YASX15RYX16 (SEQ ID NO: 9), wherein:
   X15 is selected from Q, N and I,
   X16 is selected from T, S and P, and
CDR-L-3: QQDYX17SPFT (SEQ ID NO: 10), wherein X17 is selected from A and S.

Preferably, the caninized anti-canine IL-31RA antibody, antigen-binding fragment or antigen-binding derivative according to the invention comprises heavy and light chains respectively comprising CDR-H and CDR-L with one of the following amino acid sequences sets a) to n) according to Kabat numbering:

a) 8D3-VHL/8D3-VLH:
CDR-H-1:
(SEQ ID NO: 22)
SSFIH,
CDR-H-2:
(SEQ ID NO: 23)
RIDPAFGATEYNPAFQG,
CDR-H-3:
(SEQ ID NO: 24)
YHYAASHFDA,
CDR-L-1:
(SEQ ID NO: 25)
KSSQSVTNDLT,
CDR-L-2:
(SEQ ID NO: 26)
YASQRYT,
and
CDR-L-3:
(SEQ ID NO: 27)
QQDYASPFT;
b) 8D3-clone7v2-VH/8D3-clone 7-VL:
CDR-H-1:
(SEQ ID NO: 28)
SSFIH,
CDR-H-2:
(SEQ ID NO: 29)
RIDPLQGGTEYNPVFQG,
CDR-H-3:
(SEQ ID NO: 30)
YYYAQSHFDA,
CDR-L-1:
(SEQ ID NO: 31)
KSSQSVTNDLT,
CDR-L-2:
(SEQ ID NO: 32)
YASQRYT,
and
CDR-L-3:
(SEQ ID NO: 33)
QQDYSSPFT;
c) 8D3- VH-L/8D3-VL-Ev2:
CDR-H-1:
(SEQ ID NO: 22)
SSFIH,
CDR-H-2:
(SEQ ID NO: 23)
RIDPAFGATEYNPAFQG,
CDR-H-3:
(SEQ ID NO: 24)
YHYAASHFDA,
CDR-L-1:
(SEQ ID NO: 34)
KSSQSVTNDVT,
CDR-L-2:
(SEQ ID NO: 35)
YASQRYS,
and
CDR-L-3:
(SEQ ID NO: 36)
QQDYASPFT;
d) 8D3-VH-L/8D3-VL-G:
CDR-H-1:
(SEQ ID NO: 22)
SSFIH,
CDR-H-2:
(SEQ ID NO: 23)
RIDPAFGATEYNPAFQG,
CDR-H-3:
(SEQ ID NO: 24)
YHYAASHFDA,
CDR-L-1:
(SEQ ID NO: 37)
KSSQSVTNDLT,
CDR-L-2:
(SEQ ID NO: 38)
YASQRYP,
and
CDR-L-3:
(SEQ ID NO: 39)
QQDYASPFT;
e) 8D3-VH-Lv2/8D3-VL-Ev2:
CDR-H-1:
(SEQ ID NO: 40)
SSFIH,
CDR-H-2:
(SEQ ID NO: 41)
RIDPLQGATEYNPVFQG,
CDR-H-3:
(SEQ ID NO: 42)
YHYAQSHFDA,
CDR-L-1:
(SEQ ID NO: 34)
KSSQSVTNDVT,
CDR-L-2:
(SEQ ID NO: 35)
YASQRYS,
and
CDR-L-3:
(SEQ ID NO: 36)
QQDYASPFT;
f) 8D3-VH-Lv2/8D3-VL-G:
CDR-H-1:
(SEQ ID NO: 40)
SSFIH,
CDR-H-2:
(SEQ ID NO: 41)
RIDPLQGATEYNPVFQG,
CDR-H-3:
(SEQ ID NO: 42)
YHYAQSHFDA,
CDR-L-1:
(SEQ ID NO: 37)
KSSQSVTNDLT,
CDR-L-2:
(SEQ ID NO: 38)
YASQRYP,
and
CDR-L-3:
(SEQ ID NO: 39)
QQDYASPFT;
g) 8D3-VH-N/8D3-VL-E:
CDR-H-1:
(SEQ ID NO: 43)
SSFIH,
CDR-H-2:
(SEQ ID NO: 44)
YIDPLQGGTEYNPVFQG,
CDR-H-3:
(SEQ ID NO: 45)
YYYAQSHFDA,
CDR-L-1:
(SEQ ID NO: 46)
KSSQSVTNDVT,
CDR-L-2:
(SEQ ID NO: 47)
YASIRYS,
and
CDR-L-3:
(SEQ ID NO: 48)
QQDYASPFT;
h) 8D3-VH-H/8D3-VL-Ev2:
CDR-H-1:
(SEQ ID NO: 49)
DSFIH,
CDR-H-2:
(SEQ ID NO: 50)
RIDPAQGATEYDANFQG,
CDR-H-3:
(SEQ ID NO: 51)
YYYGASHFDA,
CDR-L-1:
(SEQ ID NO: 34)
KSSQSVINDVT,
CDR-L-2:
(SEQ ID NO: 35)
YASQRYS,
and
CDR-L-3:
(SEQ ID NO: 36)
QQDYASPFT;
i) 8D3-VH-Lv2/8D3-VL-E:
CDR-H-1:
(SEQ ID NO: 40)
SSFIH,
CDR-H-2:
(SEQ ID NO: 41)
RIDPLQGATEYNPVFQG,
CDR-H-3:
(SEQ ID NO: 42)
YHYAQSHFDA,
CDR-L-1:
(SEQ ID NO: 46)
KSSQSVTNDVT,
CDR-L-2:
(SEQ ID NO: 47)
YASIRYS,
and
CDR-L-3:
(SEQ ID NO: 48)
QQDYASPFT;
j) 8D3-VH-Lv2/8D3-VL-H:
CDR-H-1:
(SEQ ID NO: 40)
SSFIH,
CDR-H-2:
(SEQ ID NO: 41)
RIDPLQGATEYNPVFQG,
CDR-H-3:
(SEQ ID NO: 42)
YHYAQSHFDA,
CDR-L-1:
(SEQ ID NO: 25)
KSSQSVTNDLT,
CDR-L-2:
(SEQ ID NO: 26)
YASQRYT,
and
CDR-L-3:
(SEQ ID NO: 27)
QQDYASPFT;
k) 8D3-VH-N/8D3-VL-Ev2:
CDR-H-1:
(SEQ ID NO: 43)
SSFIH,
CDR-H-2:
(SEQ ID NO: 44)
YIDPLQGGTEYNPVFQG,
CDR-H-3:
(SEQ ID NO: 45)
YYYAQSHFDA,
CDR-L-1:
(SEQ ID NO: 34)
KSSQSVTNDVT,
CDR-L-2:
(SEQ ID NO: 35)
YASQRYS,
and
CDR-L-3:
(SEQ ID NO: 36)
QQDYASPFT;
l) 8D3-VH-518/8D3-VL-A:
CDR-H-1:
(SEQ ID NO: 52)
DSFIH,
CDR-H-2:
(SEQ ID NO: 53)
YIDPLQGNTEYDPVFQG,
CDR-H-3:
(SEQ ID NO: 54)
YYYARSHFDA,
CDR-L-1:
(SEQ ID NO: 55)
KASQSVTNDVT,
CDR-L-2:
(SEQ ID NO: 56)
YASNRYT,
and
CDR-L-3:
(SEQ ID NO: 57)
QQDYSSPFT;
m) 8D3-VH-518/8D3-VL-Ev2:
CDR-H-1:
(SEQ ID NO: 52)
DSFIH,
CDR-H-2:
(SEQ ID NO: 53)
YIDPLQGNTEYDPVFQG,
CDR-H-3:
(SEQ ID NO: 54)
YYYARSHFDA,
CDR-L-1:
(SEQ ID NO: 34)
KSSQSVTNDVT,
CDR-L-2:
(SEQ ID NO: 35)
YASQRYS,
and
CDR-L-3:
(SEQ ID NO: 36)
QQDYASPFT;
and
n) 8D3-VH-518H/8D3-VL-A:
CDR-H-1:
(SEQ ID NO: 58)
SSFIH,
CDR-H-2:
(SEQ ID NO: 59)
YIDPLQGGTEYNPVFQG,
CDR-H-3:
(SEQ ID NO: 60)
YHYATSHFDA,
CDR-L-1:
(SEQ ID NO: 55)
KASQSVTNDVT,
CDR-L-2:
(SEQ ID NO: 56)
YASNRYT,
and
CDR-L-3:
(SEQ ID NO: 57)
QQDYSSPFT.

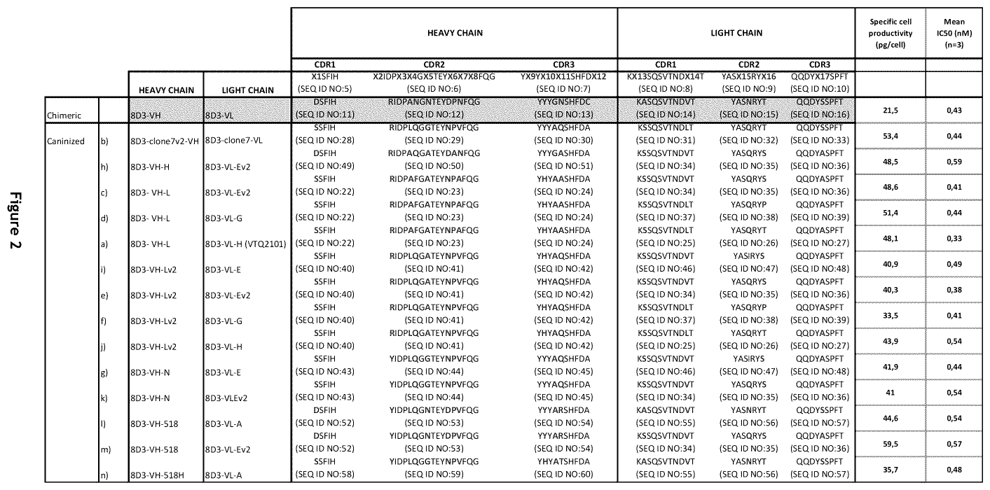

The preferred sequences of the CDRs for one chimeric and several caninized anti-canine IL-31RA antibodies according to the invention and the resulting consensus sequences of CDRs are summarized in FIG. 2.

### Variable Regions

Some positions in the FR regions of caninized versions of the 8D3 antibody were found to be important for production or maintenance of function.

Therefore, preferably, the caninized anti-canine IL-31RA antibody, antigen-binding fragment or antigen-binding derivative according to the invention has a heavy chain variable region comprising:
an amino acid selected from F, I, and L at position H67 according to Kabat numbering,
an amino acid selected from A and T at position H74 according to Kabat numbering,
an amino acid selected from A and V at position H78 according to Kabat numbering, and
an amino acid selected from S and T at position H87 according to Kabat numbering.

Also preferably, the caninized anti-canine IL-31RA antibody, antigen-binding fragment or antigen-binding derivative according to the invention has a light chain variable region comprising:
an amino acid selected from G and V at position L13 according to Kabat numbering,
an amino acid selected from A and V at position L15 according to Kabat numbering,
an amino acid selected from W and Q at position L38 according to Kabat numbering,
an amino acid selected from R and A at position L43 according to Kabat numbering,
an amino acid selected from T and H at position L49 according to Kabat numbering,
an amino acid selected from S and Y at position L67 according to Kabat numbering,
an amino acid selected from F and L at position L73 according to Kabat numbering, and
an amino acid selected from D and V at position L85 according to Kabat numbering.

More preferably, the caninized anti-canine IL-31RA antibody, antigen-binding fragment or antigen-binding derivative according to the invention has
1) a heavy chain variable region comprising:
   an amino acid selected from F, I, and L at position H67 according to Kabat numbering,
   an amino acid selected from A and T at position H74 according to Kabat numbering,
   an amino acid selected from A and V at position H78 according to Kabat numbering, and
   an amino acid selected from S and T at position H87 according to Kabat numbering, and
2) a light chain variable region comprising:
   an amino acid selected from G and V at position L13 according to Kabat numbering,
   an amino acid selected from A and V at position L15 according to Kabat numbering,
   an amino acid selected from W and Q at position L38 according to Kabat numbering,
   an amino acid selected from R and A at position L43 according to Kabat numbering,
   an amino acid selected from T and H at position L49 according to Kabat numbering,
   an amino acid selected from S and Y at position L67 according to Kabat numbering,
   an amino acid selected from F and L at position L73 according to Kabat numbering, and
   an amino acid selected from D and V at position L85 according to Kabat numbering.

Still advantageously, the caninized anti-canine IL-31RA antibody, antigen-binding fragment or antigen-binding derivative according to the invention has heavy and light chains respectively comprising CDR-H and CDR-L with the following amino acid sequences according to Kabat numbering:
CDR-H-1: X1SFIH (SEQ ID NO: 5), wherein X1 is selected from S or D, and preferably SSFIH (SEQ ID NO: 19),
CDR-H-2: X21DPX3X4GX5TEYX6X7X8FQG (SEQ ID NO: 20), wherein:
   X2 is selected from R and Y,
   X3 is selected from A and L,
   X4 is selected from F and Q,
   X5 is selected from A, N and G,
   X6 is selected from D and N,
   X7 is selected from A and P,
   X8 is selected from A, N and V,
CDR-H-3: YX9YX10X11SHFDA (SEQ ID NO: 21), wherein:
   X9 is selected from H and Y,
   X10 is selected from A and G,
   X11 is selected from A, R, T and Q.
CDR-L-1: KX13SQSVTNDX14T (SEQ ID NO: 8), wherein:
   X13 is selected from S and A,
   X14 is selected from L and V,
CDR-L-2: YASX15RYX16 (SEQ ID NO: 9), wherein:
   X15 is selected from Q, N and I,
   X16 is selected from T, S and P, and
CDR-L-3: QQDYX17SPFT (SEQ ID NO: 10), wherein X17 is selected from A and S; and with a heavy chain variable region further comprising:
   an amino acid selected from F, I, and L at position H67 according to Kabat numbering,
   an amino acid selected from A and T at position H74 according to Kabat numbering,
   an amino acid selected from A and V at position H78 according to Kabat numbering, and
   an amino acid selected from S and T at position H87 according to Kabat numbering; and
with a light chain variable region further comprising:
   an amino acid selected from G and V at position L13 according to Kabat numbering,
   an amino acid selected from A and V at position L15 according to Kabat numbering,
   an amino acid selected from W and Q at position L38 according to Kabat numbering,
   an amino acid selected from R and A at position L43 according to Kabat numbering,
   an amino acid selected from T and H at position L49 according to Kabat numbering,
   an amino acid selected from S and Y at position L67 according to Kabat numbering,
   an amino acid selected from F and L at position L73 according to Kabat numbering, and
   an amino acid selected from D and V at position L85 according to Kabat numbering.

Preferably, the caninized anti-canine IL-31RA antibody, antigen-binding fragment or antigen-binding derivative according to the invention comprises heavy and light chains respectively comprising CDR-H and CDR-L with one of the following amino acid sequences sets a) to n according to Kabat numbering:

a) 8D3-VHL/8D3-VLH:
CDR-H-1:
(SEQ ID NO: 22)
SSFIH,
CDR-H-2:
(SEQ ID NO: 23)
RIDPAFGATEYNPAFQG,
CDR-H-3:
(SEQ ID NO: 24)
YHYAASHFDA,
CDR-L-1:
(SEQ ID NO: 25)
KSSQSVTNDLT,
CDR-L-2:
(SEQ ID NO: 26)
YASQRYT,
and
CDR-L-3:
(SEQ ID NO: 27)
QQDYASPFT;
b) 8D3-clone7v2-VH/8D3-clone 7-VL:
CDR-H-1:
(SEQ ID NO: 28)
SSFIH,
CDR-H-2:
(SEQ ID NO: 29)
RIDPLQGGTEYNPVFQG,
CDR-H-3:
(SEQ ID NO: 30)
YYYAQSHFDA,
CDR-L-1:
(SEQ ID NO: 31)
KSSQSVTNDLT,
CDR-L-2:
(SEQ ID NO: 32)
YASQRYT,
and
CDR-L-3:
(SEQ ID NO: 33)
QQDYSSPFT;
c) 8D3-VH-L/8D3-VL-Ev2:
CDR-H-1:
(SEQ ID NO: 22)
SSFIH,
CDR-H-2:
(SEQ ID NO: 23)
RIDPAFGATEYNPAFQG,
CDR-H-3:
(SEQ ID NO: 24)
YHYAASHFDA,
CDR-L-1:
(SEQ ID NO: 34)
KSSQSVTNDVT,
CDR-L-2:
(SEQ ID NO: 35)
YASQRYS,
and
CDR-L-3:
(SEQ ID NO: 36)
QQDYASPFT;
d) 8D3-VH-L/8D3-VL-G:
CDR-H-1:
(SEQ ID NO: 22)
SSFIH,
CDR-H-2:
(SEQ ID NO: 23)
RIDPAFGATEYNPAFQG,
CDR-H-3:
(SEQ ID NO: 24)
YHYAASHFDA,
CDR-L-1:
(SEQ ID NO: 37)
KSSQSVTNDLT,
CDR-L-2:
(SEQ ID NO: 38)
YASQRYP,
and
CDR-L-3:
(SEQ ID NO: 39)
QQDYASPFT;
e) 8D3-VH-Lv2/8D3-VL-Ev2:
CDR-H-1:
(SEQ ID NO: 40)
SSFIH,
CDR-H-2:
(SEQ ID NO: 41)
RIDPLQGATEYNPVFQG,
CDR-H-3:
(SEQ ID NO: 42)
YHYAQSHFDA,
CDR-L-1:
(SEQ ID NO: 34)
KSSQSVTNDVT,
CDR-L-2:
(SEQ ID NO: 35)
YASQRYS,
and
CDR-L-3:
(SEQ ID NO: 36)
QQDYASPFT;
f) 8D3-VH-Lv2/8D3-VL-G:
CDR-H-1:
(SEQ ID NO: 40)
SSFIH,
CDR-H-2:
(SEQ ID NO: 41)
RIDPLQGATEYNPVFQG,
CDR-H-3:
(SEQ ID NO: 42)
YHYAQSHFDA,
CDR-L-1:
(SEQ ID NO: 37)
KSSQSVTNDLT,
CDR-L-2:
(SEQ ID NO: 38)
YASQRYP,
and
CDR-L-3:
(SEQ ID NO: 39)
QQDYASPFT;
g) 8D3-VH-N/8D3-VL-E:
CDR-H-1:
(SEQ ID NO: 43)
SSFIH,
CDR-H-2:
(SEQ ID NO: 44)
YIDPLQGGTEYNPVFQG,
CDR-H-3:
(SEQ ID NO: 45)
YYYAQSHFDA,
CDR-L-1:
(SEQ ID NO: 46)
KSSQSVTNDVT,
CDR-L-2:
(SEQ ID NO: 47)
YASIRYS,
and
CDR-L-3:
(SEQ ID NO: 48)
QQDYASPFT;
h) 8D3-VH-H/8D3-VL-Ev2:
CDR-H-1:
(SEQ ID NO: 49)
DSFIH,
CDR-H-2:
(SEQ ID NO: 50)
RIDPAQGATEYDANFQG,
CDR-H-3:
(SEQ ID NO: 51)
YYYGASHFDA,
CDR-L-1:
(SEQ ID NO: 34)
KSSQSVTNDVT,
CDR-L-2:
(SEQ ID NO: 35)
YASQRYS,
and
CDR-L-3:
(SEQ ID NO: 36)
QQDYASPFT;
i) 8D3-VH-Lv2/8D3-VL-E:
CDR-H-1:
(SEQ ID NO: 40)
SSFIH,
CDR-H-2:
(SEQ ID NO: 41)
RIDPLQGATEYNPVFQG,
CDR-H-3:
(SEQ ID NO: 42)
YHYAQSHFDA,
CDR-L-1:
(SEQ ID NO: 46)
KSSQSVTNDVT,
CDR-L-2:
(SEQ ID NO: 47)
YASIRYS,
and
CDR-L-3:
(SEQ ID NO: 48)
QQDYASPFT;
j) 8D3-VH-Lv2/8D3-VL-H:
CDR-H-1:
(SEQ ID NO: 40)
SSFIH,
CDR-H-2:
(SEQ ID NO: 41)
RIDPLQGATEYNPVFQG,
CDR-H-3:
(SEQ ID NO: 42)
YHYAQSHFDA,
CDR-L-1:
(SEQ ID NO: 25)
KSSQSVTNDLT,
CDR-L-2:
(SEQ ID NO: 26)
YASQRYT,
and
CDR-L-3:
(SEQ ID NO: 27)
QQDYASPFT;
k) 8D3-VH-N/8D3-VL-Ev2:
CDR-H-1:
(SEQ ID NO: 43)
SSFIH,
CDR-H-2:
(SEQ ID NO: 44)
YIDPLQGGTEYNPVFQG,
CDR-H-3:
(SEQ ID NO: 45)
YYYAQSHFDA,
CDR-L-1:
(SEQ ID NO: 34)
KSSQSVTNDVT,
CDR-L-2:
(SEQ ID NO: 35)
YASQRYS,
and
CDR-L-3:
(SEQ ID NO: 57)
QQDYSSPFT;
CDR-L-3:
(SEQ ID NO: 36)
QQDYASPFT;
l) 8D3-VH-518/8D3-VL-A:
CDR-H-1:
(SEQ ID NO: 52)
DSFIH,
CDR-H-2:
(SEQ ID NO: 53)
YIDPLQGNTEYDPVFQG,
CDR-H-3:
(SEQ ID NO: 54)
YYYARSHFDA,
CDR-L-1:
(SEQ ID NO: 55)
KASQSVTNDVT,
CDR-L-2:
(SEQ ID NO: 56)
YASNRYT,
and
CDR-L-3:
(SEQ ID NO: 57)
QQDYSSPFT;
m) 8D3-VH-518/8D3-VL-Ev2:
CDR-H-1:
(SEQ ID NO: 52)
DSFIH,
CDR-H-2:
(SEQ ID NO: 53)
YIDPLQGNTEYDPVFQG,
CDR-H-3:
(SEQ ID NO: 54)
YYYARSHFDA,
CDR-L-1:
(SEQ ID NO: 34)
KSSQSVTNDVT,
CDR-L-2:
(SEQ ID NO: 35)
YASQRYS,
and
CDR-L-3:
(SEQ ID NO: 36)
QQDYASPFT;
and
n) 8D3-VH-518H/8D3-VL-A:
CDR-H-1:
(SEQ ID NO: 58)
SSFIH,
CDR-H-2:
(SEQ ID NO: 59)
YIDPLQGGTEYNPVFQG,
CDR-H-3:
(SEQ ID NO: 60)
YHYATSHFDA,
CDR-L-1:
(SEQ ID NO: 55)
KASQSVTNDVT,
CDR-L-2:
(SEQ ID NO: 56)
YASNRYT,
and

and
with a heavy chain variable region further comprising:
an amino acid selected from F, I, and L at position H67 according to Kabat numbering,
an amino acid selected from A and T at position H74 according to Kabat numbering,
an amino acid selected from A and V at position H78 according to Kabat numbering, and
an amino acid selected from S and T at position H87 according to Kabat numbering; and
   with a light chain variable region further comprising:
an amino acid selected from G and V at position L13 according to Kabat numbering,
an amino acid selected from A and V at position L15 according to Kabat numbering,
an amino acid selected from W and Q at position L38 according to Kabat numbering,
an amino acid selected from R and A at position L43 according to Kabat numbering,
an amino acid selected from T and H at position L49 according to Kabat numbering,
an amino acid selected from S and Y at position L67 according to Kabat numbering,
an amino acid selected from F and L at position L73 according to Kabat numbering, and
an amino acid selected from D and V at position L85 according to Kabat numbering.
   Even more preferably, the caninized anti-canine IL-31RA antibody, antigen-binding fragment or antigen-binding derivative according to the invention comprises one of the following amino acid sequences sets a) to n):
a1) 8D3-VHL/8D3-VLH:
   a variable region of the heavy chain (VH) with at least 80%, at least 85%, at least 90%, at least 95%, at least 96%, at least 97%, at least 98%, or at least 99% identity with SEQ ID NO: 3, and/or
   a variable region of the light chain (VL) with at least 80%, at least 85%, at least 90%, at least 95%, at least 96%, at least 97%, at least 98%, or at least 99% identity with SEQ ID NO: 4;
a2) 8D3-VHL/8D3-VLHλcap:
   a variable region of the heavy chain (VH) with at least 80%, at least 85%, at least 90%, at least 95%, at least 96%, at least 97%, at least 98%, or at least 99% identity with SEQ ID NO: 3, and/or
   a variable region of the light chain (VL) with at least 80%, at least 85%, at least 90%, at least 95%, at least 96%, at least 97%, at least 98%, or at least 99% identity with SEQ ID NO: 113;
b) 8D3-clone7v2-VH/8D3-clone 7-VL:
   a variable region of the heavy chain (VH) with at least 80%, at least 85%, at least 90%, at least 95%, at least 96%, at least 97%, at least 98%, or at least 99% identity with SEQ ID NO: 61, and/or
   a variable region of the light chain (VL) with at least 80%, at least 85%, at least 90%, at least 95%, at least 96%, at least 97%, at least 98%, or at least 99% identity with SEQ ID NO: 67;
c) 8D3-VH-L/8D3-VL-Ev2:
   a variable region of the heavy chain (VH) with at least 80%, at least 85%, at least 90%, at least 95%, at least 96%, at least 97%, at least 98%, or at least 99% identity with SEQ ID NO: 3, and/or
   a variable region of the light chain (VL) with at least 80%, at least 85%, at least 90%, at least 95%, at least 96%, at least 97%, at least 98%, or at least 99% identity with SEQ ID NO: 68;
d) 8D3-VH-L/8D3-VL-G:
   a variable region of the heavy chain (VH) with at least 80%, at least 85%, at least 90%, at least 95%, at least 96%, at least 97%, at least 98%, or at least 99% identity with SEQ ID NO: 3, and/or
   a variable region of the light chain (VL) with at least 80%, at least 85%, at least 90%, at least 95%, at least 96%, at least 97%, at least 98%, or at least 99% identity with SEQ ID NO: 69;
e) 8D3-VH-Lv2/8D3-VL-Ev2:
   a variable region of the heavy chain (VH) with at least 80%, at least 85%, at least 90%, at least 95%, at least 96%, at least 97%, at least 98%, or at least 99% identity with SEQ ID NO: 62, and/or
   a variable region of the light chain (VL) with at least 80%, at least 85%, at least 90%, at least 95%, at least 96%, at least 97%, at least 98%, or at least 99% identity with SEQ ID NO: 68;
f) 8D3-VH-Lv2/8D3-VL-G:
   a variable region of the heavy chain (VH) with at least 80%, at least 85%, at least 90%, at least 95%, at least 96%, at least 97%, at least 98%, or at least 99% identity with SEQ ID NO: 62, and/or
   a variable region of the light chain (VL) with at least 80%, at least 85%, at least 90%, at least 95%, at least 96%, at least 97%, at least 98%, or at least 99% identity with SEQ ID NO: 69;
g) 8D3-VH-N/8D3-VL-E:
   a variable region of the heavy chain (VH) with at least 80%, at least 85%, at least 90%, at least 95%, at least 96%, at least 97%, at least 98%, or at least 99% identity with SEQ ID NO: 63, and/or
   a variable region of the light chain (VL) with at least 80%, at least 85%, at least 90%, at least 95%, at least 96%, at least 97%, at least 98%, or at least 99% identity with SEQ ID NO: 70;
h) 8D3-VH-H/8D3-VL-Ev2:
   a variable region of the heavy chain (VH) with at least 80%, at least 85%, at least 90%, at least 95%, at least 96%, at least 97%, at least 98%, or at least 99% identity with SEQ ID NO: 64, and/or
   a variable region of the light chain (VL) with at least 80%, at least 85%, at least 90%, at least 95%, at least 96%, at least 97%, at least 98%, or at least 99% identity with SEQ ID NO: 68;
i) 8D3-VH-Lv2/8D3-VL-E:
   a variable region of the heavy chain (VH) with at least 80%, at least 85%, at least 90%, at least 95%, at least 96%, at least 97%, at least 98%, or at least 99% identity with SEQ ID NO: 62, and/or
   a variable region of the light chain (VL) with at least 80%, at least 85%, at least 90%, at least 95%, at least 96%, at least 97%, at least 98%, or at least 99% identity with SEQ ID NO: 70;
j) 8D3-VH-Lv2/8D3-VL-H:
   a variable region of the heavy chain (VH) with at least 80%, at least 85%, at least 90%, at least 95%, at least 96%, at least 97%, at least 98%, or at least 99% identity with SEQ ID NO: 62, and/or
   a variable region of the light chain (VL) with at least 80%, at least 85%, at least 90%, at least 95%, at least 96%, at least 97%, at least 98%, or at least 99% identity with SEQ ID NO: 4;
k) 8D3-VH-N/8D3-VL-Ev2:
   a variable region of the heavy chain (VH) with at least 80%, at least 85%, at least 90%, at least 95%, at least 96%, at least 97%, at least 98%, or at least 99% identity with SEQ ID NO: 63, and/or
   a variable region of the light chain (VL) with at least 80%, at least 85%, at least 90%, at least 95%, at least 96%, at least 97%, at least 98%, or at least 99% identity with SEQ ID NO: 68;
l) 8D3-VH-518/8D3-VL-A:
   a variable region of the heavy chain (VH) with at least 80%, at least 85%, at least 90%, at least 95%, at least 96%, at least 97%, at least 98%, or at least 99% identity with SEQ ID NO: 65, and/or
   a variable region of the light chain (VL) with at least 80%, at least 85%, at least 90%, at least 95%, at least 96%, at least 97%, at least 98%, or at least 99% identity with SEQ ID NO: 71;
m) 8D3-VH-518/8D3-VL-Ev2:
   a variable region of the heavy chain (VH) with at least 80%, at least 85%, at least 90%, at least 95%, at least 96%, at least 97%, at least 98%, or at least 99% identity with SEQ ID NO: 65, and/or
   a variable region of the light chain (VL) with at least 80%, at least 85%, at least 90%, at least 95%, at least 96%, at least 97%, at least 98%, or at least 99% identity with SEQ ID NO: 68; and
n) 8D3-VH-518H/8D3-VL-A:
   a variable region of the heavy chain (VH) with at least 80%, at least 85%, at least 90%, at least 95%, at least 96%, at least 97%, at least 98%, or at least 99% identity with SEQ ID NO: 66, and/or
   a variable region of the light chain (VL) with at least 80%, at least 85%, at least 90%, at least 95%, at least 96%, at least 97%, at least 98%, or at least 99% identity with SEQ ID NO: 71.

Preferably, the anti-canine IL-31RA antibody, antigen-binding fragment or antigen-binding derivative according to the invention comprises one of the following amino acid sequences sets a) to n):
a1) 8D3-VHL/8D3-VLH:
   a variable region of the heavy chain (VH) with:

CDR-H-1:
(SEQ ID NO: 22)
SSFIH,
CDR-H-2:
(SEQ ID NO: 23)
RIDPAFGATEYNPAFQG,
CDR-H-3:
(SEQ ID NO: 24)
YHYAASHFDA,

and
   at least 80%, at least 85%, at least 90%, at least 95%, at least 96%, at least 97%, at least 98%, or at least 99% identity with SEQ ID NO: 3, and/or
   a variable region of the light chain (VL) with:

CDR-L-1:
(SEQ ID NO: 25)
KSSQSVTNDLT,
CDR-L-2:
(SEQ ID NO: 26)
YASQRYT,
CDR-L-3:
(SEQ ID NO: 27)
QQDYASPFT,

and
   at least 80%, at least 85%, at least 90%, at least 95%, at least 96%, at least 97%, at least 98%, or at least 99% identity with SEQ ID NO: 4;
a2) 8D3-VHL/8D3-VLHλcap:
   a variable region of the heavy chain (VH) with:

CDR-H-1:
(SEQ ID NO: 22)
SSFIH,
CDR-H-2:
(SEQ ID NO: 23)
RIDPAFGATEYNPAFQG,
CDR-H-3:
(SEQ ID NO: 24)
YHYAASHFDA,

and
   at least 80%, at least 85%, at least 90%, at least 95%, at least 96%, at least 97%, at least 98%, or at least 99% identity with SEQ ID NO: 3, and/or
   a variable region of the light chain (VL) with:

CDR-L-1:
(SEQ ID NO: 25)
KSSQSVTNDLT,
CDR-L-2:
(SEQ ID NO: 26)
YASQRYT,
CDR-L-3:
(SEQ ID NO: 27)
QQDYASPFT,

and
   at least 80%, at least 85%, at least 90%, at least 95%, at least 96%, at least 97%, at least 98%, or at least 99% identity with SEQ ID NO: 113;
b) 8D3-clone7v2-VH/8D3-clone 7-VL:
   a variable region of the heavy chain (VH) with:

CDR-H-1:
(SEQ ID NO: 28)
SSFIH,
CDR-H-2:
(SEQ ID NO: 29)
RIDPLQGGTEYNPVFQG,
CDR-H-3:
(SEQ ID NO: 30)
YYYAQSHFDA,

and
   at least 80%, at least 85%, at least 90%, at least 95%, at least 96%, at least 97%, at least 98%, or at least 99% identity with SEQ ID NO: 61, and/or
   a variable region of the light chain (VL) with:

CDR-L-1:
(SEQ ID NO: 31)
KSSQSVTNDLT,
CDR-L-2:
(SEQ ID NO: 32)
YASQRYT,
CDR-L-3:
(SEQ ID NO: 33)
QQDYSSPFT,

and
   at least 80%, at least 85%, at least 90%, at least 95%, at least 96%, at least 97%, at least 98%, or at least 99% identity with SEQ ID NO: 67;
c) 8D3-VH-L/8D3-VL-Ev2:
   a variable region of the heavy chain (VH) with:

CDR-H-1:
(SEQ ID NO: 22)
SSFIH,
CDR-H-2:
(SEQ ID NO: 23)
RIDPAFGATEYNPAFQG,
CDR-H-3:
(SEQ ID NO: 24)
YHYAASHFDA,

and
   at least 80%, at least 85%, at least 90%, at least 95%, at least 96%, at least 97%, at least 98%, or at least 99% identity with SEQ ID NO: 3, and/or
   a variable region of the light chain VL with:

CDR-L-1:
(SEQ ID NO: 34)
KSSQSVTNDVT,
CDR-L-2:
(SEQ ID NO: 35)
YASQRYS,
CDR-L-3:
(SEQ ID NO: 36)
QQDYASPFT,

and
   at least 80%, at least 85%, at least 90%, at least 95%, at least 96%, at least 97%, at least 98%, or at least 99% identity with SEQ ID NO: 68;
d) 8D3-VH-L/8D3-VL-G:
   a variable region of the heavy chain (VH) with:

CDR-H-1:
(SEQ ID NO: 22)
SSFIH,
CDR-H-2:
(SEQ ID NO: 23)
RIDPAFGATEYNPAFQG,
CDR-H-3:
(SEQ ID NO: 24)
YHYAASHFDA,

and
   at least 80%, at least 85%, at least 90%, at least 95%, at least 96%, at least 97%, at least 98%, or at least 99% identity with SEQ ID NO: 3, and/or
   a variable region of the light chain (VL) with:

CDR-L-1:
(SEQ ID NO: 37)
KSSQSVTNDLT,
CDR-L-2:
(SEQ ID NO: 38)
YASQRYP,
CDR-L-3:
(SEQ ID NO: 39)
QQDYASPFT,

and
   at least 80%, at least 85%, at least 90%, at least 95%, at least 96%, at least 97%, at least 98%, or at least 99% identity with SEQ ID NO: 69;
e) 8D3-VH-Lv2/8D3-VL-Ev2:
   a variable region of the heavy chain (VH) with:

CDR-H-1:
(SEQ ID NO: 40)
SSFIH,
CDR-H-2:
(SEQ ID NO: 41)
RIDPLQGATEYNPVFQG,
CDR-H-3:
(SEQ ID NO: 42)
YHYAQSHFDA,

and
   at least 80%, at least 85%, at least 90%, at least 95%, at least 96%, at least 97%, at least 98%, or at least 99% identity with SEQ ID NO: 62, and/or
   a variable region of the light chain (VL) with:

CDR-L-1:
(SEQ ID NO: 34)
KSSQSVTNDVT,
CDR-L-2:
(SEQ ID NO: 35)
YASQRYS,
CDR-L-3:
(SEQ ID NO: 36)
QQDYASPFT,

and
   at least 80%, at least 85%, at least 90%, at least 95%, at least 96%, at least 97%, at least 98%, or at least 99% identity with SEQ ID NO: 68;
f) 8D3-VH-Lv2/8D3-VL-G:
   a variable region of the heavy chain (VH) with:

CDR-H-1:
(SEQ ID NO: 40)
SSFIH,
CDR-H-2:
(SEQ ID NO: 41)
RIDPLQGATEYNPVFQG,
CDR-H-3:
(SEQ ID NO: 42)
YHYAQSHFDA,

and
   at least 80%, at least 85%, at least 90%, at least 95%, at least 96%, at least 97%, at least 98%, or at least 99% identity with SEQ ID NO: 62, and/or
   a variable region of the light chain (VL) with:

CDR-L-1:
(SEQ ID NO: 37)
KSSQSVTNDLT,
CDR-L-2:
(SEQ ID NO: 38)
YASQRYP,
CDR-L-3:
(SEQ ID NO: 39)
QQDYASPFT,

and
   at least 80%, at least 85%, at least 90%, at least 95%, at least 96%, at least 97%, at least 98%, or at least 99% identity with SEQ ID NO: 69;
g) 8D3-VH-N/8D3-VL-E:
   a variable region of the heavy chain (VH) with:

CDR-H-1:
(SEQ ID NO: 43)
SSFIH,
CDR-H-2:
(SEQ ID NO: 44)
YIDPLQGGTEYNPVFQG,
CDR-H-3:
(SEQ ID NO: 45)
YYYAQSHFDA,

and
   at least 80%, at least 85%, at least 90%, at least 95%, at least 96%, at least 97%, at least 98%, or at least 99% identity with SEQ ID NO: 63, and/or
   a variable region of the light chain (VL) with:

CDR-L-1:
(SEQ ID NO: 46)
KSSQSVTNDVT,
CDR-L-2:
(SEQ ID NO: 47)
YASIRYS,
CDR-L-3:
(SEQ ID NO: 48)
QQDYASPFT,

and
   at least 80%, at least 85%, at least 90%, at least 95%, at least 96%, at least 97%, at least 98%, or at least 99% identity with SEQ ID NO: 70;
h) 8D3-VH-H/8D3-VL-Ev2:
   a variable region of the heavy chain (VH) with:

CDR-H-1:
(SEQ ID NO: 49)
DSFIH,
CDR-H-2:
(SEQ ID NO: 50)
RIDPAQGATEYDANFQG,
CDR-H-3:
(SEQ ID NO: 51)
YYYGASHFDA,

and
   at least 80%, at least 85%, at least 90%, at least 95%, at least 96%, at least 97%, at least 98%, or at least 99% identity with SEQ ID NO: 64, and/or
   a variable region of the light chain (VL) with:

CDR-L-1:
(SEQ ID NO: 34)
KSSQSVTNDVT,
CDR-L-2:
(SEQ ID NO: 35)
YASQRYS,
CDR-L-3:
(SEQ ID NO: 36)
QQDYASPFT,

and
   at least 80%, at least 85%, at least 90%, at least 95%, at least 96%, at least 97%, at least 98%, or at least 99% identity with SEQ ID NO: 68;
i) 8D3-VH-Lv2/8D3-VL-E:
   a variable region of the heavy chain (VH) with:

CDR-H-1:
(SEQ ID NO: 40)
SSFIH,
CDR-H-2:
(SEQ ID NO: 41)
RIDPLQGATEYNPVFQG,
CDR-H-3:
(SEQ ID NO: 42)
YHYAQSHFDA,

and
   at least 80%, at least 85%, at least 90%, at least 95%, at least 96%, at least 97%, at least 98%, or at least 99% identity with SEQ ID NO: 62, and/or
   a variable region of the light chain (VL) with:

CDR-L-1:
(SEQ ID NO: 46)
KSSQSVTNDVT,
CDR-L-2:
(SEQ ID NO: 47)
YASIRYS,
CDR-L-3:
(SEQ ID NO: 48)
QQDYASPFT,

and
   at least 80%, at least 85%, at least 90%, at least 95%, at least 96%, at least 97%, at least 98%, or at least 99% identity with SEQ ID NO: 70;
j) 8D3-VH-Lv2/8D3-VL-H:
   a variable region of the heavy chain (VH) with:

CDR-H-1:
(SEQ ID NO: 40)
SSFIH,
CDR-H-2:
(SEQ ID NO: 41)
RIDPLQGATEYNPVFQG,
CDR-H-3:
(SEQ ID NO: 42)
YHYAQSHFDA,

and
   at least 80%, at least 85%, at least 90%, at least 95%, at least 96%, at least 97%, at least 98%, or at least 99% identity with SEQ ID NO: 62, and/or
   a variable region of the light chain (VL) with:

CDR-L-1:
(SEQ ID NO: 25)
KSSQSVTNDLT,
CDR-L-2:
(SEQ ID NO: 26)
YASQRYT,
CDR-L-3:
(SEQ ID NO: 27)
QQDYASPFT,

and
   at least 80%, at least 85%, at least 90%, at least 95%, at least 96%, at least 97%, at least 98%, or at least 99% identity with SEQ ID NO: 4;
k) 8D3-VH-N/8D3-VL-Ev2:
   a variable region of the heavy chain (VH) with:

CDR-H-1:
(SEQ ID NO: 43)
SSFIH,
CDR-H-2:
(SEQ ID NO: 44)
YIDPLQGGTEYNPVFQG,
CDR-H-3:
(SEQ ID NO: 45)
YYYAQSHFDA,

and
   at least 80%, at least 85%, at least 90%, at least 95%, at least 96%, at least 97%, at least 98%, or at least 99% identity with SEQ ID NO: 63, and/or
   a variable region of the light chain (VL with:

CDR-L-1:
(SEQ ID NO: 34)
KSSQSVTNDVT,
CDR-L-2:
(SEQ ID NO: 35)
YASQRYS,
CDR-L-3:
(SEQ ID NO: 36)
QQDYASPFT,

and
   at least 80%, at least 85%, at least 90%, at least 95%, at least 96%, at least 97%, at least 98%, or at least 99% identity with SEQ ID NO: 68;
l) 8D3-VH-518/8D3-VL-A:
   a variable region of the heavy chain (VH) with:

CDR-H-1:
(SEQ ID NO: 52)
DSFIH,
CDR-H-2:
(SEQ ID NO: 53)
YIDPLQGNTEYDPVFQG,
CDR-H-3:
(SEQ ID NO: 54)
YYYARSHFDA,

and
   at least 80%, at least 85%, at least 90%, at least 95%, at least 96%, at least 97%, at least 98%, or at least 99% identity with SEQ ID NO: 65, and/or
   a variable region of the light chain (VL) with:

CDR-L-1:
(SEQ ID NO: 55)
KASQSVTNDVT,
CDR-L-2:
(SEQ ID NO: 56)
YASNRYT,
CDR-L-3:
(SEQ ID NO: 57)
QQDYSSPFT,

and
   at least 80%, at least 85%, at least 90%, at least 95%, at least 96%, at least 97%, at least 98%, or at least 99% identity with SEQ ID NO: 71;
m) 8D3-VH-518/8D3-VL-Ev2:
   a variable region of the heavy chain (VH) with:

CDR-H-1:
(SEQ ID NO: 52)
DSFIH,
CDR-H-2:
(SEQ ID NO: 53)
YIDPLQGNTEYDPVFQG,
CDR-H-3:
(SEQ ID NO: 54)
YYYARSHFDA,

and
   at least 80%, at least 85%, at least 90%, at least 95%, at least 96%, at least 97%, at least 98%, or at least 99% identity with SEQ ID NO: 65, and/or
   a variable region of the light chain (VL) with:

CDR-L-1:
(SEQ ID NO: 34)
KSSQSVTNDVT,
CDR-L-2:
(SEQ ID NO: 35)
YASQRYS,
and
CDR-L-3:
(SEQ ID NO: 36)
QQDYASPFT;

and
   at least 80%, at least 85%, at least 90%, at least 95%, at least 96%, at least 97%, at least 98%, or at least 99% identity with SEQ ID NO: 68; and
n) 8D3-VH-518H/8D3-VL-A:
   a variable region of the heavy chain (VH) with:

CDR-H-1:
(SEQ ID NO: 58)
SSFIH,
CDR-H-2:
(SEQ ID NO: 59)
YIDPLQGGTEYNPVFQG,
CDR-H-3:
(SEQ ID NO: 60)
YHYATSHFDA,

and
   at least 80%, at least 85%, at least 90%, at least 95%, at least 96%, at least 97%, at least 98%, or at least 99% identity with SEQ ID NO: 66, and/or
   a variable region of the light chain (VL) with:

CDR-L-1:
(SEQ ID NO: 55)
KASQSVTNDVT,
CDR-L-2:
(SEQ ID NO: 56)
YASNRYT,
CDR-L-3:
(SEQ ID NO: 57)
QQDYSSPFT,

and
   at least 80%, at least 85%, at least 90%, at least 95%, at least 96%, at least 97%, at least 98%, or at least 99% identity with SEQ ID NO: 71;
preferably wherein:
   the heavy chain variable region of said antibody, antigen-binding fragment or antigen-binding derivative further comprises:
      an amino acid selected from F, I, and L at position H67 according to Kabat numbering,
      an amino acid selected from A and T at position H74 according to Kabat numbering,
      an amino acid selected from A and V at position H78 according to Kabat numbering, and
      an amino acid selected from S and T at position H87 according to Kabat numbering; and
   the light chain variable region of said antibody further comprises:
      an amino acid selected from G and V at position L13 according to Kabat numbering,
      an amino acid selected from A and V at position L15 according to Kabat numbering,
      an amino acid selected from W and Q at position L38 according to Kabat numbering,
      an amino acid selected from R and A at position L43 according to Kabat numbering,
      an amino acid selected from T and H at position L49 according to Kabat numbering,
      an amino acid selected from S and Y at position L67 according to Kabat numbering,
      an amino acid selected from F and L at position L73 according to Kabat numbering, and
      an amino acid selected from D and V at position L85 according to Kabat numbering.

The most preferred anti-canine IL-31RA antibody, antigen-binding fragment or antigen-binding derivative according to the invention comprises one of the following amino acid sequences sets a) to n):
a1) 8D3-VHL/8D3-VLH:
a variable region of the heavy chain (VH) with SEQ ID NO: 3, and
a variable region of the light chain (VL) with SEQ ID NO: 4;
a2) 8D3-VHL/8D3-VLHλcap:
a variable region of the heavy chain (VH) with SEQ ID NO: 3, and
a variable region of the light chain (VL) with SEQ ID NO: 113;
b) 8D3-clone7v2-VH/8D3-clone 7-VL:
a variable region of the heavy chain (VH) with SEQ ID NO: 61, and
a variable region of the light chain (VL) with SEQ ID NO: 67;
c) 8D3-VH-L/8D3-VL-Ev2:
a variable region of the heavy chain (VH) with SEQ ID NO: 3, and
a variable region of the light chain (VL) with SEQ ID NO: 68;
d) 8D3-VH-L/8D3-VL-G:
a variable region of the heavy chain (VH) with SEQ ID NO: 3, and
a variable region of the light chain (VL) with SEQ ID NO: 69;
e) 8D3-VH-Lv2/8D3-VL-Ev2:
a variable region of the heavy chain (VH) with SEQ ID NO: 62, and
a variable region of the light chain (VL) with SEQ ID NO: 68;
f) 8D3-VH-Lv2/8D3-VL-G:
a variable region of the heavy chain (VH) with SEQ ID NO: 62, and
a variable region of the light chain (VL) with SEQ ID NO: 69;
g) 8D3-VH-N/8D3-VL-E:
a variable region of the heavy chain (VH) with SEQ ID NO: 63, and
a variable region of the light chain (VL) with SEQ ID NO: 70;
h) 8D3-VH-H/8D3-VL-Ev2:
a variable region of the heavy chain (VH) with SEQ ID NO: 64, and
a variable region of the light chain (VL) with SEQ ID NO: 68;
i) 8D3-VH-Lv2/8D3-VL-E:
a variable region of the heavy chain (VH) with SEQ ID NO: 62, and
a variable region of the light chain (VL) with SEQ ID NO: 70;
j) 8D3-VH-Lv2/8D3-VL-H:
a variable region of the heavy chain (VH) with SEQ ID NO: 62, and
a variable region of the light chain (VL) with SEQ ID NO: 4;
k) 8D3-VH-N/8D3-VL-Ev2:
a variable region of the heavy chain (VH) with SEQ ID NO: 63, and
a variable region of the light chain (VL) with SEQ ID NO: 68;
l) 8D3-VH-518/8D3-VL-A:
a variable region of the heavy chain (VH) with SEQ ID NO: 65, and
a variable region of the light chain (VL) with SEQ ID NO: 71;
m) 8D3-VH-518/8D3-VL-Ev2:
a variable region of the heavy chain (VH) with SEQ ID NO: 65, and
a variable region of the light chain (VL) with SEQ ID NO: 68; and
n) 8D3-VH-518H/8D3-VL-A:
a variable region of the heavy chain (VH) with SEQ ID NO: 66, and
a variable region of the light chain (VL) with SEQ ID NO: 71.

Table 3 below summarizes the preferred sequences of the variable regions of the heavy chain and associated CDRs of caninized anti-canine IL-31RA antibodies according to the invention:

| | | Corresponding CDRs (Kabat |
|---|---|---|
| VH name | Sequence | numbering) |
| 8D3-VHL | EVTLQESGPGLVKPSQTLSLTCVAS | CDR-H-1: SSFIH (SEQ ID NO: 22), |
| | GFSIKSSFIHWLRQRPGRGLEWIGRI | CDR-H-2: RIDPAFGATEYNPAFQG (SEQ |
| | DPAFGATEYNPAFQGRFSITADTAK | ID NO: 23), |
| | NQASLQLSSMTTEDSAVYYCARYHY | CDR-H-3: YHYAASHFDA (SEQ ID NO: 24) |
| | AASHFDAWGQGTLVTVSS | |
| | (SEQ ID NO: 3) | |
| 8D3- | EVTLQESGPGLVKPSQTLSLTCVAS | CDR-H-1: SSFIH (SEQ ID NO: 28), |
| clone7v2-VH | GFSIKSSFIHWLRQRPGRGLEWIGRI | CDR-H-2: RIDPLQGGTEYNPVFQG (SEQ |
| | DPLQGGTEYNPVFQGRISITADTTK | ID NO: 29), |
| | NQVSLQLSSMTTEDTAVYYCARYYY | CDR-H-3: YYYAQSHFDA (SEQ ID NO: |
| | AQSHFDAWGQGTLVTVSS | 30), |
| | (SEQ ID NO: 61) | |
| 8D3-VH-Lv2 | EVTLQESGPGLVKPSQTLSLTCVAS | CDR-H-1: SSFIH (SEQ ID NO: 40), |
| | GFSIKSSFIHWLRQRPGRGLEWIGRI | CDR-H-2: RIDPLQGATEYNPVFQG (SEQ |
| | DPLQGATEYNPVFQGRLSITADTAK | ID NO: 41), |
| | NQVSLQLSSMTTEDTAVYYCARYH | CDR-H-3: YHYAQSHFDA (SEQ ID NO: 42) |
| | YAQSHFDAWGQGTLVTVSS | |
| | (SEQ ID NO: 62) | |
| 8D3-VH-N | EVTLQESGPGLVKPSQTLSLTCVAS | CDR-H-1: SSFIH (SEQ ID NO: 43), |
| | GFSIKSSFIHWLRQRPGRGLEWIGYI | CDR-H-2: YIDPLQGGTEYNPVFQG (SEQ |
| | DPLQGGTEYNPVFQGRISITADTAK | ID NO: 44), |
| | NQVSLQLSSMTTEDTAVYYCARYYY | CDR-H-3: YYYAQSHFDA (SEQ ID NO: 45) |
| | AQSHFDAWGQGTLVTVSS | |
| | (SEQ ID NO: 63) | |
| 8D3-VH-H | EVTLQESGPGLVKPSQTLSLTCVAS | CDR-H-1: DSFIH (SEQ ID NO: 49), |
| | GFSIKDSFIHWLRQRPGRGLEWIGR | CDR-H-2: RIDPAQGATEYDANFQG (SEQ |
| | IDPAQGATEYDANFQGRISITADTA | ID NO: 50), |
| | KNQASLQLSSMTTEDTAVYYCARYY | CDR-H-3: YYYGASHFDA (SEQ ID NO: 51) |
| | YGASHFDAWGQGTLVTVSS | |
| | (SEQ ID NO: 64) | |
| 8D3-VH-518 | VTLQESGPGLVKPSQTLSLTCVASG | CDR-H-1: DSFIH (SEQ ID NO: 52), |
| | FSIKDSFIHWLRQRPGRGLEWIGYID | |
| | PLQGNTEYDPVFQGRLSITADTAKN | CDR-H-2: YIDPLQGNTEYDPVFQG (SEQ |
| | | ID NO: 53), |
| | QVSLQLSSMTTEDTAVYYCARYYYA | CDR-H-3: YYYARSHFDA (SEQ ID NO: 54) |
| | RSHFDAWGQGTLVTVSS | |
| | (SEQ ID NO: 65) | |
| 8D3-VH-518H | EVTLQESGPGLVKPSQTLSLTCVAS | CDR-H-1: SSFIH (SEQ ID NO: 58), |
| | GFSIKSSFIHWLRQRPGRGLEWIGYI | CDR-H-2: YIDPLQGGTEYNPVFQG (SEQ |
| | DPLQGGTEYNPVFQGRLSITADTAK | ID NO: 59), |
| | NQVSLQLSSMTTEDTAVYYCARYH | CDR-H-3: YHYATSHFDA (SEQ ID NO: 60) |
| | YATSHFDAWGQGTLVTVSS | |
| | (SEQ ID NO: 66) | |

Table 4 below summarizes the preferred sequences of the variable regions of the light chain and associated CDRs of caninized anti-canine IL-31RA antibodies according to the invention:

| | | Corresponding CDRs (Kabat |
|---|---|---|
| VL name | Sequence | numbering) |
| 8D3-VLH | RIVMTQSPGSLAGSVGESVSINCKSS | CDR-L-1: KSSQSVTNDLT (SEQ ID NO: |
| | QSVTNDLTWYQQKPGEAPKVLITY | 25), |
| | ASQRYTGVPARFSGSGYGTDFTLTI | CDR-L-2: YASQRYT (SEQ ID NO: 26), |
| | NNLQAEDVGDYFCQQDYASPFTFG | and |
| | QGTKLEIK | CDR-L-3: QQDYASPFT (SEQ ID NO: 27) |
| | (SEQ ID NO: 4) | |
| 8D3-VLHAcap | RIVMTQSPGSLAGSVGESVSINCKSS | CDR-L-1: KSSQSVTNDLT (SEQ ID NO: |
| | QSVTNDLTWYQQKPGEAPKVLITY | 25), |
| | ASQRYTGVPARFSGSGYGTDFTLTI | CDR-L-2: YASQRYT (SEQ ID NO: 26), |
| | NNLQAEDVGDYFCQQDYASPFTFG | and |
| | GGTKLTVLG (SEQ ID NO: 113) | CDR-L-3: QQDYASPFT (SEQ ID NO: 27) |
| 8D3-clone 7- | RIVMTQSPGSLAGSAGESVSINCKSS | CDR-L-1: KSSQSVTNDLT (SEQ ID NO: |
| VL | QSVTNDLTWYQQKPGEAPKVLIHY | 31), |
| | ASQRYTGVPARFSGSGYGTDFTLTI | CDR-L-2: YASQRYT (SEQ ID NO: 32), |
| | NNLQAEDVGVYFCQQDYSSPFTFG | and |
| | QGTKLEIK | CDR-L-3: QQDYSSPFT (SEQ ID NO: 33) |
| | (SEQ ID NO: 67) | |
| 8D3-VL-Ev2 | RIVMTQSPGSLAGSVGESVSINCKSS | CDR-L-1: KSSQSVTNDVT (SEQ ID NO: |
| | QSVTNDVTWYQQKPGEAPKVLIHY | 34), |
| | ASQRYSGVPARFSGSGYGTDFTLTI | CDR-L-2: YASQRYS (SEQ ID NO: 35), and |
| | NNLQAEDVGDYFCQQDYASPFTFG | CDR-L-3: QQDYASPFT (SEQ ID NO: 36); |
| | QGTKLEIK | |
| | (SEQ ID NO: 68) | |
| 8D3-VL-G | RIVMTQSPGSLAGSVGESVSINCKSS | CDR-L-1: KSSQSVTNDLT (SEQ ID NO: |
| | QSVTNDLTWYQQKPGEAPKVLIHY | 37), |
| | ASQRYPGVPARFSGSGYGTDFTLTI | CDR-L-2: YASQRYP (SEQ ID NO: 38), |
| | NNLQAEDVGDYFCQQDYASPFTFG | and |
| | QGTKLEIK | CDR-L-3: QQDYASPFT (SEQ ID NO: 39) |
| | (SEQ ID NO: 69) | |
| 8D3-VL-E | RIVMTQSPGSLAVSVGESVSINCKSS | CDR-L-1: KSSQSVTNDVT (SEQ ID NO: |
| | QSVTNDVTWYQWKPGEAPKVLITY | 46), |
| | ASIRYSGVPARFSGSGYGTDFTLTIN | CDR-L-2: YASIRYS (SEQ ID NO: 47), and |
| | NLQAEDVGDYFCQQDYASPFTFGQ | CDR-L-3: QQDYASPFT (SEQ ID NO: 48); |
| | GTKLEIK | |
| | (SEQ ID NO: 70) | |
| 8D3-VL-A | RIVMTQSPGSLAGSAGESVSINCKAS | CDR-L-1: KASQSVINDVT (SEQ ID NO: |
| | QSVTNDVTWYQQKPGERPKVLIHY | 55), |
| | ASNRYTGVPARFSGSGSGTDFTFTI | CDR-L-2: YASNRYT (SEQ ID NO: 56), |
| | NNLQAEDVGDYFCQQDYSSPFTFG | and |
| | QGTKLEIK | CDR-L-3: QQDYSSPFT (SEQ ID NO: 57) |
| | (SEQ ID NO: 71) | |

The most preferred sequences of the variable regions for caninized antibodies according to the present invention are those of 8D3-VHL/8D3-VLH and 8D3-VHL/8D3-VLHλcap, as described above in Tables 3 and 4.

### Constant Regions of Chimeric and Caninized Antibodies

The constant regions of chimeric antibodies according to the invention are preferably canine constant regions and the constant regions of caninized antibodies according to the invention are canine constant regions.

The anti-canine IL-31RA antibodies of the present invention may be of several canine isotypes, according to the nature of their constant region and which correspond to the canine immunoglobulins IgG, IgA, IgM, IgE and IgD.

Advantageously, the anti-canine IL-31RA antibody according to the present invention is of canine isotype IgG, and more preferably of canine isotype IgGB. In canine, there are four IgG heavy chains referred to as A, B, C and D. These heavy chains represent four different subclasses of dog IgG, which are referred to as IgGA, IgGB, IgGC and IgGD. Each IgG heavy chain consist of one variable domain (VH) and three constant domains referred to as CH1, CH2 and CH3. The CH1 domain is connected to the CH2 domain via an amino acid sequence referred to as the “hinge” or alternatively as the “hinge region”.

The DNA and amino acid sequences of these four heavy chains were first identified by Tang et al. 2001. The amino acid and DNA sequences for these heavy chains are also available from the GenBank data bases. For example, the amino acid sequence of IgGA heavy chain has the gene accession number AF354264.1, IgGB has accession number AF354265.1, IgGC has accession number AF354266.1, and IgGD has accession number AF354267.1. Canine antibodies also contain two types of light chains, kappa and lambda. The amino acid sequence of these light chains can be obtained from UniProtKB or IMGT databases. For example, the kappa light chain amino acid sequence has the accession number F1NY2 in UniProtKB and the lambda light chain amino acid sequence can be found in IMGT at the following address; http://www.imgt.org/IMGTrepertoire/index.php?section=LocusGenesftrepertoire=genetable&species=dog&group=IGLC.

Thus, in one advantageous embodiment the heavy chain of the anti-canine IL-31RA antibody according the invention comprises a wild type canine IgGB constant region (SEQ ID NO: 72):

ASTTAPSVFPLAPSCGSTSGSTVALACLVSGYFPEPVTVSWNSGS
LTSGVHTFPSVLQSSGLYSLSSMVTVPSSRWPSETFTCNVAHPAS
KTKVDKPVPKRENGRVPRPPDCPKCPAPEMLGGPSVFIFPPKPKD
TLLIARTPEVTCVVVDLDPEDPEVQISWFVDGKQMQTAKTQPREE
QFNGTYRVVSVLPIGHQDWLKGKQFTCKVNNKALPSPIERTISKA
RGQAHQPSVYVLPPSREELSKNTVSLTCLIKDFFPPDIDVEWQSN
GQQEPESKYRTTPPQLDEDGSYFLYSKLSVDKSRWQRGDTFICAV
MHEALHNHYTQESLSHSPG

The anti-canine IL-31RA antibody according to the invention can also be optimized for absence or reduction of certain effector functions, and especially in order to avoid or reduce ADCC (antibody-dependent cell cytotoxicity), ADCP (antibody-dependent phagocytosis); the cell-mediated reactions wherein nonspecific cytotoxic cells that express FcγRs recognize bound antibody on a target cell and subsequently cause lysis of the target cell and/or CDC (complement-dependent cytotoxicity); activation of the classical complement pathway by binding of protein C1q to bound antibody on a target cell and subsequently resulting in target cell lysis. Thus, its heavy chain's constant region may particularly comprise mutations for reducing its affinity for the Fcγ receptor(s) or complement protein(s) to which its isotype binds. Generally, any antibody IgG isotype can be used in which the Fc portion is modified (e.g., by introducing 1, 2, 3, 4, 5 or more amino acid substitutions) to minimize or eliminate binding to Fc receptors (see, e.g., WO 2003/101485, the disclosure of which is herein incorporated by reference). Assays such as cell-based assays, to assess Fc receptor binding are well known in the art, and are described in, e.g. WO 2003/101485.

For example, it has been shown that several types of mutations in the Fc region of canine IgGB region may influence binding to Fcγ receptor(s) (e.g., any one or more of CD16A, CD16B, CD32A, CD32B and/or CD64) or complement protein(s) (e.g., C1q) and result in “Fc silent” antibodies that have minimal interaction with effector cells or complement protein(s).

In the context of the present invention, the preferred mutants are in the CH2 domain of canine IgGB (SEQ ID NO: 72) heavy chain's constant regions and comprise at least one of the following mutations (numbering of the positions being according to Eu numbering nomenclature (Edelman et al. 1969):
hinge mutation K228P, to avoid Fab arm exchange and improve manufacturability of the molecule,
CH2 mutation(s) M234A and/or L235A reducing binding to Fc gamma receptors, thus avoiding ADCP and ADCC,
CH2 mutation D265A, which reduces binding to Fc gamma receptors, thus avoiding ADCP and ADCC,
CH2 mutation P329G or P329A, which reduces binding of C1q complement protein;
CH2 mutation N297G or N297A, which destroys the N-glycosylation site NST in the Fc, resulting in a non-glycosylated (aglycosylated) antibody with reduced ADCP and ADCC activities, or
any combination thereof.

In particular, preferred mutant canine IgGB heavy chain's constant regions may contain:
the three mutations M234A-L235A-P329G in order to reduce binding to Fc gamma receptors and avoid ADCP and ADCC (mutations M234A-L235A) and reduce binding of C1q complement protein (P329G), such as the sequence (SEQ ID NO: 73):

ASTTAPSVFPLAPSCGSTSGSTVALACLVSGYFPEPVTVSWNSGS
LTSGVHTFPSVLQSSGLYSLSSMVTVPSSRWPSETFTCNVAHPAS
KTKVDKPVPKRENGRVPRPPDCPKCPAPEAAGGPSVFIFPPKPKD
TLLIARTPEVTCVVVDLDPEDPEVQISWFVDGKQMQTAKTQPREE
QFNGTYRVVSVLPIGHQDWLKGKQFTCKVNNKALGSPIERTISKA
RGQAHQPSVYVLPPSREELSKNTVSLTCLIKDFFPPDIDVEWQSN
GQQEPESKYRTTPPQLDEDGSYFLYSKLSVDKSRWQRGDTFICAV
MHEALHNHYTQESLSHSPG

the three mutations M234A-L235A-P329A in order to reduce binding to Fc gamma receptors and avoid ADCP and ADCC (mutations M234A-L235A) and reduce binding of C1q complement protein (P329A), such as the sequence (SEQ ID NO: 74):

ASTTAPSVFPLAPSCGSTSGSTVALACLVSGYFPEPVTVSWNSGS
LTSGVHTFPSVLQSSGLYSLSSMVTVPSSRWPSETFTCNVAHPAS
KTKVDKPVPKRENGRVPRPPDCPKCPAPEAAGGPSVFIFPPKPKD
TLLIARTPEVTCVVVDLDPEDPEVQISWFVDGKQMQTAKTQPREE
QFNGTYRVVSVLPIGHQDWLKGKQFTCKVNNKALASPIERTISKA
RGQAHQPSVYVLPPSREELSKNTVSLTCLIKDFFPPDIDVEWQSN
GQQEPESKYRTTPPQLDEDGSYFLYSKLSVDKSRWQRGDTFICAV
MHEALHNHYTQESLSHSPG

the four mutations K228P-M234A-L235A-P329G in order to improve manufacturability (K228P), reduce binding to Fc gamma receptors and avoid ADCP and ADCC (mutations M234A-L235A) and reduce binding of C1q complement protein (P329G), such as the sequence (SEQ ID NO: 75):

ASTTAPSVFPLAPSCGSTSGSTVALACLVSGYFPEPVTVSWNSGS
LTSGVHTFPSVLQSSGLYSLSSMVTVPSSRWPSETFTCNVAHPAS
KTKVDKPVPKRENGRVPRPPDCPPCPAPEAAGGPSVFIFPPKPKD
TLLIARTPEVTCVVVDLDPEDPEVQISWFVDGKQMQTAKTQPREE
QFNGTYRVVSVLPIGHQDWLKGKQFTCKVNNKALGSPIERTISKA
RGQAHQPSVYVLPPSREELSKNTVSLTCLIKDFFPPDIDVEWQSN
GQQEPESKYRTTPPQLDEDGSYFLYSKLSVDKSRWQRGDTFICAV
MHEALHNHYTQESLSHSPG

the four mutations K228P-M234A-L235A-P329A in order to improve manufacturability (K228P), reduce binding to Fc gamma receptors and avoid ADCP and ADCC (mutations M234A-L235A) and reduce binding of C1q complement protein (P329G), such as the sequence (SEQ ID NO: 76):

ASTTAPSVFPLAPSCGSTSGSTVALACLVSGYFPEPVTVSWNSGS
LTSGVHTFPSVLQSSGLYSLSSMVTVPSSRWPSETFTCNVAHPAS
KTKVDKPVPKRENGRVPRPPDCPPCPAPEAAGGPSVFIFPPKPKD
TLLIARTPEVTCVVVDLDPEDPEVQISWFVDGKQMQTAKTQPREE
QFNGTYRVVSVLPIGHQDWLKGKQFTCKVNNKALASPIERTISKA
RGQAHQPSVYVLPPSREELSKNTVSLTCLIKDFFPPDIDVEWQSN
GQQEPESKYRTTPPQLDEDGSYFLYSKLSVDKSRWQRGDTFICAV
MHEALHNHYTQESLSHSPG

the two mutations N297G-P329G for having a non-glycosylated (aglycosylated) Fc with reduced ADCP and ADCC activities (N297G) and reduce binding of C1q complement protein (P329G), such as sequence (SEQ ID NO: 77):

ASTTAPSVFPLAPSCGSTSGSTVALACLVSGYFPEPVTVSWNSGS
LTSGVHTFPSVLQSSGLYSLSSMVTVPSSRWPSETFTCNVAHPAS
KTKVDKPVPKRENGRVPRPPDCPKCPAPEMLGGPSVFIFPPKPKD
TLLIARTPEVTCVVVDLDPEDPEVQISWFVDGKQMQTAKTQPREE
QFGGTYRVVSVLPIGHQDWLKGKQFTCKVNNKALGSPIERTISKA
RGQAHQPSVYVLPPSREELSKNTVSLTCLIKDFFPPDIDVEWQSN
GQQEPESKYRTTPPQLDEDGSYFLYSKLSVDKSRWQRGDTFICAV
MHEALHNHYTQESLSHSPG

the two mutations N297G-P329A for having a non-glycosylated (aglycosylated) Fc with reduced ADCP and ADCC activities (N297G) and reduce binding of C1q complement protein (P329A), such as sequence (SEQ ID NO: 78):

ASTTAPSVFPLAPSCGSTSGSTVALACLVSGYFPEPVTVSWNSGS
LTSGVHTFPSVLQSSGLYSLSSMVTVP SSRWPSETFTCNVAHPA
SKTKVDKPVPKRENGRVPRPPDCPKCPAPEMLGGPSVFIFPPKPK
DTLLIARTPEVTCVVVDLDPEDPEVQISWFVDGKQMQTAKTQPRE
EQFGGTYRVVSVLPIGHQDWLKGKQFTCKVNNKALASPIERTISK
ARGQAHQPSVYVLPPSREELSKNTVSLTCLIKDFFPPDIDVEWQS
NGQQEPESKYRTTPPQLDEDGSYFLYSKLSVDKSRWQRGDTFICA
VMHEALHNHYTQESLSHSPG

the two mutations N297A-P329G for having a non-glycosylated (aglycosylated) Fc with reduced ADCP and ADCC activities (N297A) and reduce binding of C1 q complement protein (P329G), such as sequence (SEQ ID NO: 79):

ASTTAPSVFPLAPSCGSTSGSTVALACLVSGYFPEPVTVSWNSGSLTSGVHTFPSVLQSSGLYSLSSMVTVPSSRWPSETFT
CNVAHPASKTKVDKPVPKRENGRVPRPPDCPKCPAPEMLGGPSVFIFPPKPKDTLLIARTPEVTCVVVDLDPEDPEVQISWF
VDGKQMQTAKTQPREEQFAGTYRVVSVLPIGHQDWLKGKQFTCKVNNKALGSPIERTISKARGQAHQPSVYVLPPSREEL
SKNTVSLTCLIKDFFPPDIDVEWQSNGQQEPESKYRTTPPQLDEDGSYFLYSKLSVDKSRWQRGDTFICAVMHEALHNHYT
QESLSHSPG

the two mutations N297A-P329A for having a non-glycosylated (aglycosylated) Fc with reduced ADCP and ADCC activities (N297A) and reduce binding of C1 q complement protein (P329A), such as sequence (SEQ ID NO: 80):

ASTTAPSVFPLAPSCGSTSGSTVALACLVSGYFPEPVTVSWNSGSLTSGVHTFPSVLQSSGLYSLSSMVTVPSSRWPSETFT
CNVAHPASKTKVDKPVPKRENGRVPRPPDCPKCPAPEMLGGPSVFIFPPKPKDTLLIARTPEVTCVVVDLDPEDPEVQISWF
VDGKQMQTAKTQPREEQFAGTYRVVSVLPIGHQDWLKGKQFTCKVNNKALASPIERTISKARGQAHQPSVYVLPPSREELS
KNTVSLTCLIKDFFPPDIDVEWQSNGQQEPESKYRTTPPQLDEDGSYFLYSKLSVDKSRWQRGDTFICAVMHEALHNHYTQ
ESLSHSPG

the two mutations K228P-N297G for improving manufacturability (K228P) and having a non-glycosylated (aglycosylated) Fc with reduced ADCP and ADCC activities (N297G), such as sequence (SEQ ID NO: 81):

ASTTAPSVFPLAPSCGSTSGSTVALACLVSGYFPEPVTVSWNSGSLTSGVHTFPSVLQSSGLYSLSSMVTVPSSRWPSETFT
CNVAHPASKTKVDKPVPKRENGRVPRPPDCPPCPAPEMLGGPSVFIFPPKPKDTLLIARTPEVTCVVVDLDPEDPEVQISWF
VDGKQMQTAKTQPREEQFGGTYRVVSVLPIGHQDWLKGKQFTCKVNNKALPSPIERTISKARGQAHQPSVYVLPPSREEL
SKNTVSLTCLIKDFFPPDIDVEWQSNGQQEPESKYRTTPPQLDEDGSYFLYSKLSVDKSRWQRGDTFICAVMHEALHNHYT
QESLSHSPG

the two mutations K228P-N297A for improving manufacturability (K228P) and having a non-glycosylated (aglycosylated) Fc with reduced ADCP and ADCC activities (N297A), such as sequence (SEQ ID NO: 82):

ASTTAPSVFPLAPSCGSTSGSTVALACLVSGYFPEPVTVSWNSGSLTSGVHTFPSVLQSSGLYSLSSMVTVPSSRWPSETFT
CNVAHPASKTKVDKPVPKRENGRVPRPPDCPPCPAPEMLGGPSVFIFPPKPKDTLLIARTPEVTCVVVDLDPEDPEVQISWF
VDGKQMQTAKTQPREEQFAGTYRVVSVLPIGHQDWLKGKQFTCKVNNKALPSPIERTISKARGQAHQPSVYVLPPSREELS
KNTVSLTCLIKDFFPPDIDVEWQSNGQQEPESKYRTTPPQLDEDGSYFLYSKLSVDKSRWQRGDTFICAVMHEALHNHYTQ
ESLSHSPG

the two mutations D265A-N297G for reducing binding to both FcγR and C1q, such as sequence (SEQ ID NO: 83):

ASTTAPSVFPLAPSCGSTSGSTVALACLVSGYFPEPVTVSWNSGSLTSGVHTFPSVLQSSGLYSLSSMVTVPSSRWPSETFT
CNVAHPASKTKVDKPVPKRENGRVPRPPDCPKCPAPEMLGGPSVFIFPPKPKDTLLIARTPEVTCVVVALDPEDPEVQISWF
VDGKQMQTAKTQPREEQFGGTYRVVSVLPIGHQDWLKGKQFTCKVNNKALPSPIERTISKARGQAHQPSVYVLPPSREEL
SKNTVSLTCLIKDFFPPDIDVEWQSNGQQEPESKYRTTPPQLDEDGSYFLYSKLSVDKSRWQRGDTFICAVMHEALHNHYT
QESLSHSPG

the two mutations D265A-N297A for reducing binding to both FcγR and C1q, such as sequence (SEQ ID NO: 84):

ASTTAPSVFPLAPSCGSTSGSTVALACLVSGYFPEPVTVSWNSGSLTSGVHTFPSVLQSSGLYSLSSMVTVPSSRWPSETFT
CNVAHPASKTKVDKPVPKRENGRVPRPPDCPKCPAPEMLGGPSVFIFPPKPKDTLLIARTPEVTCVVVALDPEDPEVQISWF
VDGKQMQTAKTQPREEQFAGTYRVVSVLPIGHQDWLKGKQFTCKVNNKALPSPIERTISKARGQAHQPSVYVLPPSREELS
KNTVSLTCLIKDFFPPDIDVEWQSNGQQEPESKYRTTPPQLDEDGSYFLYSKLSVDKSRWQRGDTFICAVMHEALHNHYTQ
ESLSHSPG

the single mutation N297G for reducing binding to both FcγR and C1q, such as sequence (SEQ ID NO: 85):

ASTTAPSVFPLAPSCGSTSGSTVALACLVSGYFPEPVTVSWNSGSLTSGVHTFPSVLQSSGLYSLSSMVTVPSSRWPSETFT
CNVAHPASKTKVDKPVPKRENGRVPRPPDCPKCPAPEMLGGPSVFIFPPKPKDTLLIARTPEVTCVVVDLDPEDPEVQISWF
VDGKQMQTAKTQPREEQFGGTYRVVSVLPIGHQDWLKGKQFTCKVNNKALPSPIERTISKARGQAHQPSVYVLPPSREEL
SKNTVSLTCLIKDFFPPDIDVEWQSNGQQEPESKYRTTPPQLDEDGSYFLYSKLSVDKSRWQRGDTFICAVMHEALHNHYT
QESLSHSPG

the single mutation N297A for reducing binding to both FcγR and C1q, such as sequence (SEQ ID NO: 86):

ASTTAPSVFPLAPSCGSTSGSTVALACLVSGYFPEPVTVSWNSGSLTSGVHTFPSVLQSSGLYSLSSMVTVPSSRWPSETFT
CNVAHPASKTKVDKPVPKRENGRVPRPPDCPKCPAPEMLGGPSVFIFPPKPKDTLLIARTPEVTCVVVDLDPEDPEVQISWF
VDGKQMQTAKTQPREEQFAGTYRVVSVLPIGHQDWLKGKQFTCKVNNKALPSPIERTISKARGQAHQPSVYVLPPSREELS
KNTVSLTCLIKDFFPPDIDVEWQSNGQQEPESKYRTTPPQLDEDGSYFLYSKLSVDKSRWQRGDTFICAVMHEALHNHYTQ
ESLSHSPG

The amino acid sequences SEQ ID NO:72 to SEQ ID NO:86 defined above correspond to the entire IgGB constant region. In each of these sequences, amino acids 99 to 334 correspond to the Fc fragment.

Also, in one advantageous embodiment, the constant region of the light chain of the anti-canine IL-31RA antibody according the invention is a lambda type of sequence: GQPKASPSVTLFPPSSEELGANKATLVCLISDFYPSGVTVAWKADGSPVTQGVETTKPSKQSNNKYAASSYLSLTPDKWKS HSSFSCLVTHECSTVEKKVAPAECS (SEQ ID NO: 87), or a kappa type of sequence: RNDAQPAVYLFQPSPDQLHTGSASVVCLLNSFYPKDINVKWKVDGVIQDTGIQESVTEQDKDSTYSLSSTLTMSSTEYLSHE LYSCEITHKSLPSTLIKSFQRSEC (SEQ ID NO: 88), and preferably a lambda type.

In particular, one even more advantageous embodiment concerns a chimeric or caninized isotype IgGB anti-canine IL-31RA antibody, comprising a lambda type light chain constant region associated with a heavy chain constant region of canine IgGB type with the four mutations K228P-M234A-L235A-P329G or three mutations M234A-L235A-P329G, and preferably three mutations M234A-L235A-P329G.

Alternatively, or in combination with mutations intended to limit the antibody's effector (ADCC and/or CDC) functions, the glycosylation of the antibody heavy chain constant region may be altered in order to reduce its affinity for the Fcγ receptor(s) or C1q complement protein(s) to which normally binds.

### Preferred Full-Length Antibodies

By adding the preferred heavy and light chain constant regions, the preferred complete amino acid sequences of the antibodies according to the present invention are obtained, as described in Table 5 below:

| Heavy chain | | Light chain | |
|---|---|---|---|
| variable region | Constant region | variable region | Constant region |

Preferred chimeric full-length antibodies

| | | | |
|---|---|---|---|
| 8D3 VH chimeric | Canine IgGB WT | 8D3 VL chimeric | Kappa type |
| (SEQ ID NO: 17) | (SEQ ID NO: 72) | (SEQ ID NO: 18) | (SEQ ID NO: 88) |
| 8D3 VH chimeric | Canine IgGB | 8D3 VL chimeric | Kappa type |
| (SEQ ID NO: 17) | M234A-L235A-P329G | (SEQ ID NO: 18) | (SEQ ID NO: 88) |
| | (SEQ ID NO: 73) | | |

Preferred caninized full-length antibodies

| | | | |
|---|---|---|---|
| 8D3-VHL | Canine IgGB WT | 8D3-VLH | Kappa type |
| (SEQ ID NO: 3) | (SEQ ID NO: 72) | (SEQ ID NO: 4) | (SEQ ID NO: 88) |
| 8D3-VHL | Canine IgGB M234A- | 8D3-VLH | Kappa type |
| (SEQ ID NO: 3) | L235A-P329G | (SEQ ID NO: 4) | (SEQ ID NO: 88) |
| | (SEQ ID NO: 73) | | |
| 8D3-VHL | Canine IgGB WT | 8D3-VLHλcap | Lambda type |
| (SEQ ID NO: 3) | (SEQ ID NO: 72) | (SEQ ID NO: 113) | (SEQ ID NO: 87) |
| 8D3-VHL | Canine IgGB M234A- | 8D3- VLHλcap | Lambda type |
| (SEQ ID NO: 3) | L235A-P329G | (SEQ ID NO: 113) | (SEQ ID NO: 87) |
| | (SEQ ID NO: 73) | | |
| 8D3-clone7v2-VH | Canine IgGB WT | 8D3-clone 7-VL | Kappa type |
| (SEQ ID NO: 61) | (SEQ ID NO: 72) | (SEQ ID NO: 67) | (SEQ ID NO: 88) |
| 8D3-clone7v2-VH | Canine IgGB M234A- | 8D3-clone 7-VL | Kappa type |
| (SEQ ID NO: 61) | L235A-P329G | (SEQ ID NO: 67) | (SEQ ID NO: 88) |
| | (SEQ ID NO: 73) | | |
| 8D3-VH-L | Canine IgGB WT | 8D3-VL-Ev2 | Kappa type |
| (SEQ ID NO: 3) | (SEQ ID NO: 72) | (SEQ ID NO: 68) | (SEQ ID NO: 88) |
| 8D3-VH-L | Canine IgGB M234A- | 8D3-VL-Ev2 | Kappa type |
| (SEQ ID NO: 3) | L235A-P329G | (SEQ ID NO: 68) | (SEQ ID NO: 88) |
| | (SEQ ID NO: 73) | | |
| 8D3-VH-L | Canine IgGB WT | 8D3-VL-G | Kappa type |
| (SEQ ID NO: 3) | (SEQ ID NO: 72) | (SEQ ID NO: 69) | (SEQ ID NO: 88) |
| 8D3-VH-L | Canine IgGB M234A- | 8D3-VL-G | Kappa type |
| (SEQ ID NO: 3) | L235A-P329G | (SEQ ID NO: 69) | (SEQ ID NO: 88) |
| | (SEQ ID NO: 73) | | |
| 8D3-VH-Lv2 | Canine IgGB WT | 8D3-VL-Ev2 | Kappa type |
| (SEQ ID NO: 62) | (SEQ ID NO: 72) | (SEQ ID NO: 68) | (SEQ ID NO: 88) |
| 8D3-VH-Lv2 | Canine lgGB M234A- | 8D3-VL-Ev2 | Kappa type |
| (SEQ ID NO: 62) | L235A-P329G | (SEQ ID NO: 68) | (SEQ ID NO: 88) |
| | (SEQ ID NO: 73) | | |
| 8D3-VH-Lv2 | Canine IgGB WT | 8D3-VL-G | Kappa type |
| (SEQ ID NO: 62) | (SEQ ID NO: 72) | (SEQ ID NO: 69) | (SEQ ID NO: 88) |
| 8D3-VH-Lv2 | Canine IgGB M234A- | 8D3-VL-G | Kappa type |
| (SEQ ID NO: 62) | L235A-P329G | (SEQ ID NO: 69) | (SEQ ID NO: 88) |
| | (SEQ ID NO: 73) | | |
| 8D3-VH-N | Canine IgGB WT | 8D3-VL-E | Kappa type |
| (SEQ ID NO: 63) | (SEQ ID NO: 72) | (SEQ ID NO: 70) | (SEQ ID NO: 88) |
| 8D3-VH-N | Canine IgGB M234A- | 8D3-VL-E | Kappa type |
| (SEQ ID NO: 63) | L235A-P329G | (SEQ ID NO: 70) | (SEQ ID NO: 88) |
| | (SEQ ID NO: 73) | | |
| 8D3-VH-H | Canine IgGB WT | 8D3-VL-Ev2 | Kappa type |
| (SEQ ID NO: 64) | (SEQ ID NO: 72) | (SEQ ID NO: 68) | (SEQ ID NO: 88) |
| 8D3-VH-H | Canine IgGB M234A- | 8D3-VL-Ev2 | Kappa type |
| (SEQ ID NO: 64) | L235A-P329G | (SEQ ID NO: 68) | (SEQ ID NO: 88) |
| | (SEQ ID NO: 73) | | |
| 8D3-VH-Lv2 | Canine IgGB WT | 8D3-VL-E | Kappa type |
| (SEQ ID NO: 62) | (SEQ ID NO: 72) | (SEQ ID NO: 70) | (SEQ ID NO: 88) |
| 8D3-VH-Lv2 | Canine IgGB M234A- | 8D3-VL-E | Kappa type |
| (SEQ ID NO: 62) | L235A-P329G | (SEQ ID NO: 70) | (SEQ ID NO: 88) |
| | (SEQ ID NO: 73) | | |
| 8D3-VH-Lv2 | Canine IgGB WT | 8D3-VLH | Kappa type |
| (SEQ ID NO: 62) | (SEQ ID NO: 72) | (SEQ ID NO: 4) | (SEQ ID NO: 88) |
| 8D3-VH-Lv2 | Canine IgGB M234A- | 8D3-VLH | Kappa type |
| (SEQ ID NO: 62) | L235A-P329G | (SEQ ID NO: 4) | (SEQ ID NO: 88) |
| | (SEQ ID NO: 73) | | |
| 8D3-VH-N | Canine IgGB WT | 8D3-VL-Ev2 | Kappa type |
| (SEQ ID NO: 63) | (SEQ ID NO: 72) | (SEQ ID NO: 68) | (SEQ ID NO: 88) |
| 8D3-VH-N | Canine IgGB M234A- | 8D3-VL-Ev2 | Kappa type |
| (SEQ ID NO: 63) | L235A-P329G | (SEQ ID NO: 68) | (SEQ ID NO: 88) |
| | (SEQ ID NO: 73) | | |
| 8D3-VH-518 | Canine IgGB WT | 8D3-VL-A | Kappa type |
| (SEQ ID NO: 65) | (SEQ ID NO: 72) | (SEQ ID NO: 71) | (SEQ ID NO: 88) |
| 8D3-VH-518 | Canine IgGB M234A- | 8D3-VL-A | Kappa type |
| (SEQ ID NO: 65) | L235A-P329G | (SEQ ID NO: 71) | (SEQ ID NO: 88) |
| | (SEQ ID NO: 73) | | |
| 8D3-VH-518 | Canine IgGB WT | 8D3-VL-Ev2 | Kappa type |
| (SEQ ID NO: 65) | (SEQ ID NO: 72) | (SEQ ID NO: 68) | (SEQ ID NO: 88) |
| 8D3-VH-518 | Canine IgGB M234A- | 8D3-VL-Ev2 | Kappa type |
| (SEQ ID NO: 65) | L235A-P329G | (SEQ ID NO: 68) | (SEQ ID NO: 88) |
| | (SEQ ID NO: 73) | | |
| 8D3-VH-518H | Canine IgGB WT | 8D3-VL-A | Kappa type |
| (SEQ ID NO: 66) | (SEQ ID NO: 72) | (SEQ ID NO: 71) | (SEQ ID NO: 88) |
| 8D3-VH-518H | Canine IgGB M234A- | 8D3-VL-A | Kappa type |
| (SEQ ID NO: 66) | L235A-P329G | (SEQ ID NO: 71) | (SEQ ID NO: 88) |
| | (SEQ ID NO: 73) | | |

### Antigen-Binding Fragments and Antigen-Binding Derivatives

By “antigen-binding fragment” is meant an antibody fragment retaining the antigen-binding domain and thus having the same antigen specificity as the original antibody as well as similar potency for inhibiting the canine IL-31RA signaling pathway. An antigen-binding fragment according to the invention is advantageously selected from the group consisting of a Fab fragment, a Fab′ fragment, a Fab′-SH fragment, a F(ab)2 fragment, a F(ab)′2 fragment, an Fv fragment, a Heavy chain Ig (a llama or camel Ig), a VHH fragment, a single domain Fv and a single-chain antibody fragment.

By “antigen-binding derivative” of an antibody is meant at least one antibody fragment according to the invention as defined above linked to at least one peptide or polypeptide or other polymers. Such antigen-binding derivatives are notable selected in the group consisting of scFv, a dsFV, a minibody, a diabody, a tribody, a kappa body, an IgNAR, scFv-Fc derivatives of formula VH-linker-VL-Fc, VL-linker-VH-Fc, and ScFab-Fc derivatives of formula LC-linker-HC, wherein VH and VL correspond to the variable domains of the heavy and light chains, respectively, HC and LC correspond to the entire heavy and light chains respectively, Fc corresponds to the Fc fragment (consisting of the heavy chain constant region excluding the CH1 domain and upper hinge region, i.e. the Fc fragment consists of the lower hinge region and the constant domains CH2 and CH3 or CH2 to CH4 (depending on the isotype)), and “linker” corresponds to a flexible peptide linker that ensures proper folding and optimal activity of the derivative.

Some of the antigen-binding derivatives may be designed for extending half-life such as by fusion with an Fc fragment (such as scFv-Fc or scFab-Fc derivatives mentioned in the preceding paragraph), canine serum albumin, VHH anti-canine serum albumin or by grafting an alternative scaffold directed against canine serum albumin or other chemical polymers known to extend in vivo half-life such as polyethyleneglycol (PEG), or polypeptides such as PAS polypeptides comprising repetitive sequences of proline, alanine and/or serine or such as unstructured hydrophilic, biodegradable protein polymers named “XTEN”.

Preferred derivatives include scFv-Fc derivatives of formula VH-linker-VL-Fc, VL-linker-VH-Fc, and ScFab-Fc derivatives of formula LC-linker-HC, wherein VH and VL correspond to the variable domains of the heavy and light chains, respectively, HC and LC correspond to the entire heavy and light chains respectively, Fc corresponds to the Fc fragment (consisting of the heavy chain constant region excluding the CH1 domain and upper hinge region, i.e. the Fc fragment consists of the lower hinge region and the constant domains CH2 and CH3 or CH2 to CH4 (depending on the isotype)), and “linker” corresponds to a flexible peptide linker that ensures proper folding and optimal activity of the derivative.

The VH or VL domains of any scFv-Fc or ScFab-Fc derivative defined above may notably comprise CDR regions selected from any of those disclosed above for the antibodies according to the invention, in particular the chimeric or caninized antibodies according to the invention, and/or be selected from any VH or VL disclosed above for the antibodies according to the invention, in particular the chimeric or caninized antibodies according to the invention.

In any scFv-Fc derivative defined above, the Fc fragment is preferably a canine Fc fragment, in particular one of those corresponding respectively to amino acids 99 to 334 of SEQ ID NO:72 to SEQ ID NO:86, in particular amino acids 99 to 334 of SEQ ID NO: 73 and SEQ ID NO:75, and most preferably amino acids 99 to 334 of SEQ ID NO: 75.

In any scFv-Fc or ScFab-Fc derivative defined above, the VL domain may comprise a kappa or lambda J gene, preferably a lambda J gene.

Typically, suitable peptide linkers for scFv and the above scFv-Fc or scFab-Fc derivatives are 1 to 60 amino acids long peptides composed of amino acid residues such as glycine, serine, threonine, asparagine, alanine and/or proline. Preferred linkers in the context of this invention comprise, or consist essentially of, or consist of:
3 to 25 amino acids, in particular 15 to 25 amino acids, mainly glycine and serine (e.g. 1, 2 3 or 4 repetitions of GSG, GGGS (SEQ ID NO: 114), GGGGS (SEQ ID NO: 115), GSGSG (SEQ ID NO: 116), or SGSGS (SEQ ID NO: 117), such as GGGGSGGGGSGGGGS (SEQ ID NO: 118) or 1 or 2 repetitions of GSGSGSGSGS (SEQ ID NO: 119)) or glycine, serine and threonine (e.g. 1, 2 3 or 4 repetitions of GSTSG (SEQ ID NO: 120) or SGTGS (SEQ ID NO: 121) or 1 repetition of GSTSGGGSGGGSGGGGSS (SEQ ID NO: 95; GS18)) or glycine, serine, and threonine and/or alanine (e.g. 1, 2 3 or 4 repetitions of GAS or GTS). Preferred peptide linkers of 3 to 25 amino acids, in particular 15 to 25 amino acids, include those comprising one or more repetition(s) of GGGS (SEQ ID NO: 114), GGGGS (SEQ ID NO: 115), or both, such as GSTSGGGSGGGSGGGGSS (SEQ ID NO: 95; GS18) or GGGGSGGGGSGGGGS (SEQ ID NO: 118), in particular GSTSGGGSGGGSGGGGSS (SEQ ID NO: 95; GS18).
These linkers are particularly useful for scFv-Fc derivatives of formula VH-linker-VL-Fc or VL-linker-VH-Fc described above; or
40 to 60 amino acids, in particular 45 to 55 amino acids, mainly glycine and serine (such as 8 to 15 repetitions of GGGS (SEQ ID NO: 114), GGGGS (SEQ ID NO: 115), GSGSG (SEQ ID NO: 116), or SGSGS (SEQ ID NO: 117) or 4 to 6 repetitions of GSGSGSGSGS (SEQ ID NO: 119)), glycine, serine and threonine (e.g. 8 to 12 repetitions of GSTSG (SEQ ID NO: 120) or SGTGS (SEQ ID NO: 121) or 2, 3 or 4 repetitions of GSTSGGGSGGGSGGGGSS (SEQ ID NO: 95; GS18)), glycine, serine, threonine and alanine (such as GSSGSGSGSTGTSSSGTGTSAGTTGTSASTSGSGSGGGGGSGGGGSAGG (SEQ ID NO: 96; GS50). Preferred peptide linkers of 40 to 60 amino acids, in particular 45 to 55 amino acids, include those rising one or more repetition(s) of GGGS (SEQ ID NO: 114), GGGGS (SEQ ID NO: 115), or both, such as GSSGSGSGSTGTSSSGTGTSAGTTGTSASTSGSGSGGGGGSGGGGSAGG (SEQ ID NO: 96; GS50).
These linkers are particularly useful for scFab-Fc derivatives of formula LC-linker-HC described above.

Preferred scFv-Fc derivatives of formula VH-linker-VL-Fc are those comprising any one of SEQ ID NO: 106 to 109, each fused in C-terminal with an Fc fragment, notably any canine Fc fragment described herein. Particularly preferred scFv-Fc derivatives of formula VH-linker-VL-Fc are those comprising or consisting of amino acid sequences SEQ ID NO: 97 to SEQ ID NO: 100.

Preferred scFv-Fc derivatives of formula VL-linker-VH-Fc are those comprising any one of SEQ ID NO: 111 to 112, each fused in C-terminal with an Fc fragment, notably any canine Fc fragment described herein. Particularly preferred scFv-Fc derivatives of formula VL-linker-VH-Fc are those comprising or consisting of amino acid sequences SEQ ID NO: 102 or SEQ ID NO: 103.

Preferred scFab-Fc derivatives of formula LC-linker-HC are those comprising SEQ ID NO: 110 fused in C-terminal with an Fc fragment, notably any canine Fc fragment described herein. A particularly preferred scFab-Fc derivative of formula LC-linker-HC is the scFab-Fc derivative comprising or consisting of amino acid sequences SEQ ID NO: 101.

As for antibody fragments as defined above, the antigen-binding derivatives of the present invention retain its ability to recognize canine IL-31RA and to inhibit the signaling pathway activated by the binding of canine IL-31 to canine IL-31RA with equivalent or the same level of that of the original antibody, and preferably at the advantageous IC50 as defined herein.

### Bispecific Antibodies

The above-mentioned antigen-binding fragments and antigen-binding derivatives may be used to produce bispecific antibodies, which also represent an aspect of the present invention.

In another aspect, the present invention is thus related to a bispecific antibody comprising an antigen-binding fragment or antigen-binding derivative as described above, and an antigen-binding fragment or antigen-binding derivative directed to one other target relevant for treating atopic dermatitis.

The skilled person well knows how to product bi- or multi-specific antibodies and especially as described in Fan et al. 2015.

The antibody, antigen-binding fragment or antigen-binding derivative thereof according to the invention, as well as bi- or multi-specific antibody as described above, may be produced from any host cell, any transgenic non-human animal or transgenic plant described in the present description, and notably below in the section concerning the nucleic acids, vectors, host cells, transgenic non-human animals or transgenic plants according to the invention.

### Nucleic Acids Encoding an Antibody, Antigen-Binding Fragment or Antigen-Binding Derivative

The present invention also relates to a nucleic acid (herein also called nucleic or nucleotide sequence or polynucleotide) or a combination of two nucleic acids, encoding the antibodies, antigen-binding fragment or antigen-binding derivative thereof or encoding the bispecific antibody according to the invention, all as described above.

All the different nucleic sequences, because of degeneration of the genetic code, encoding a particular amino acid sequence are within the scope of the invention.

In particular, the sequence of a nucleic acid according to the invention may be optimized to promote the expression thereof in a host cell, a transgenic non-human animal of interest. Indeed, there are in general several three-nucleotide combinations encoding the same amino acid (except for methionine and tryptophan), called synonymous codons. However, some of these combinations are in general used preferentially by a cell or a given organism (this is referred to as genetic code usage bias).

This preference depends notably on the producing organism from which the cell is derived. Consequently, when a protein derived from one or more organisms is produced in a heterologous organism or a cell of such a heterologous organism, it may be useful to modify the nucleic sequence encoding the protein to use mainly the preferred codons of the heterologous organism. Data are available in the literature concerning the use of codons preferred by different species and a person skilled in the art knows how to optimize the expression of a given protein in a heterologous organism or a cell of a heterologous organism.

The present invention also relates to a nucleic sequence encoding the heavy and/or light chain constant regions of an antibody, antigen-binding fragment or antigen-binding derivative thereof according to the invention as described above.

### Vectors

The present invention also relates to a vector comprising a nucleic acid or combination of nucleic acids according to the invention. Such a vector comprises the elements necessary for the expression of said nucleic sequence(s), and notably a promoter, a transcription initiation codon, termination sequences, and suitable transcription regulatory sequences. These elements vary according to the host used for the expression and are easily selected by persons skilled in the art based on their general knowledge. In particular, for a vector designed for expression in eukaryotic cells, the vector advantageously comprises a Kozak consensus sequence, i.e., a conserved sequence found at the translation start site of eukaryotic messenger RNA, around the AUG start codon (generally GCCGCC(A/G)CCATGG (GCCGCCXCCATGG, where X is A or G; SEQ ID NO: 92), the translation initiation codon being underlined). The vector can notably be a plasmid or viral vector. It is used to clone or express the nucleic acids according to the invention.

The one skilled in the art would routinely know and found vectors able to be used in the context of the invention, including the transcription unit to be used.

### Host Cells, Transgenic Non-Human Animals, Transgenic Plants

The present invention also relates to a host cell, a transgenic non-human animal or a transgenic plant comprising at least one nucleic acid or combination of nucleic acids according to the invention or a vector according to the invention.

The host cell may be of prokaryotic or eukaryotic origin, and may in particular be selected from bacterial, insect, plant, fungus, yeast or mammalian cells. The antibody, antigen-binding fragment or antigen-binding derivative thereof according to the invention may then be produced by culturing the host cell under suitable conditions.

A host cell according to the invention can notably be obtained by transforming a cell line by the expression vector(s) for the heavy and light chains of an antibody, antigen-binding fragment or antigen-binding derivative thereof according to the invention, and separating the various cell clones obtained. The transformed cell line is preferably of eukaryotic origin, and may in particular be selected from insects, plants, yeast, or mammalian cells. Suitable cell lines available for antibody production notably include lines selected from Chinese hamster ovary (CHO) cells, Baby hamster kidney (BHK) fibroblasts, murine lymphoid cell lines (NSO and Sp2/O), Human embryonic kidney (HEK293) cells and Human embryonic retinal (PER.C6) cells.

A transgenic non-human animal according to the invention may be obtained by directly injecting the gene(s) of interest (here, the sequences encoding the heavy and light chains of the antibody) into a fertilized egg (Gordon et al.—1980). A transgenic non-human animal may also be obtained by introducing the gene(s) of interest (here, the sequences encoding the heavy and light chains of the antibody) into an embryonic stem cell and preparing the animal by a chimera aggregation method or a chimera injection method (see Manipulating the Mouse Embryo, A Laboratory Manual, of Brigid Hogan et al., Second edition, Cold Spring Harbor Laboratory Press (1994); Gene Targeting, A Practical Approach, by Alexandra L. Joyner, IRL Press at Oxford University Press (1993)). A transgenic non-human animal may also be obtained by a cloning technique in which a nucleus, into which the gene(s) of interest (here, the sequences encoding the heavy and light chains of the antibody) has/have been introduced, is transplanted into an enucleated egg (Ryan et al., 1997; Cibelli et al., 1998, WO00/26357). A transgenic non-human animal producing an antibody of interest can be prepared by the methods above. The antibody may then be accumulated in the transgenic animal and harvested, notably from the animal's milk or eggs. For producing antibodies in the milk of transgenic non-human animals, preparation methods are notably described in WO90/04036, WO95/17085, WOO1/26455, WO2004/050847, WO2005/033281, WO2007/048077. Methods for purifying proteins of interest from milk are also known (see WOO1/26455, WO2007/106078). The transgenic non-human animals of interest notably include mice, rabbits, rats, goats, bovines (notably cows), and poultry (notably chicken).

A transgenic plant according to the invention may be selected from any plant allowing antibody production. Numerous antibodies have already been produced in transgenic plants and the technologies required for obtaining a transgenic plant expressing an antibody of interest and for recovering the antibody are well-known to a person skilled in the art (see Stoger et al., 2002, Fisher et al., 2003 Schillberg et al., 2005). It is also possible to influence the glycosylation obtained in the plants or any other necessary addition or modification in order to be similar to that of natural canine antibodies.

### Therapeutic Uses

The present invention also relates to an antibody, antigen-binding fragment or antigen-binding derivative thereof according to the invention, for use as a medicinal product.

The antibody, functional fragment or antigen-binding derivative thereof according to the invention is used in the treatment or prevention of the following diseases, for which a role of IL-31 has been established: atopic dermatitis, contact dermatitis, psoriasis, allergic asthma, inflammatory bowel disease, neurodegeneration, chronic rhinosinusitis, and eosinophilic diseases, preferably in the treatment or prevention of canine atopic dermatitis.

In a first embodiment, the antibody, antigen-binding fragment or antigen-binding derivative thereof according to the invention is advantageously used in the treatment or prevention of itch and/or inflammatory skin due to atopic dermatitis in dogs.

In a second embodiment, the antibody, antigen-binding fragment or antigen-binding derivative thereof according to the invention is advantageously used in the treatment or prevention of itch and/or inflammatory skin due to allergies in dogs.

The present invention also concerns the use of an antibody, antigen-binding fragment or antigen-binding derivative thereof according to the invention for preparing a medicinal product for treating or preventing of itch and/or inflammatory skin due to atopic dermatitis and/or allergies in dogs.

The present invention also concerns the use of an antibody, antigen-binding fragment or antigen-binding derivative thereof according to the invention in the treatment or prevention of itch and/or inflammatory skin due to atopic dermatitis and/or allergies in dogs.

The present invention also concerns a method for treating or preventing itch and/or inflammatory skin due to atopic dermatitis and/or allergies in dogs, comprising administering to dogs an effective amount of an antibody, antigen-binding fragment or antigen-binding derivative thereof according to the invention.

The present invention also concerns a pharmaceutical composition comprising an antibody, antigen-binding fragment or antigen-binding derivative thereof according to the invention for use in the treatment or prevention of itch and/or inflammatory skin due to atopic dermatitis and/or allergies in dogs.

By “treatment” is meant an improvement, observed at the clinical or biochemical level, of the subject's disease.

By “prevention” is meant the fact of preventing or delaying the onset of, or of decreasing the intensity of, the clinical or biochemical manifestations associated with the disease.

Persons skilled in the art know, on the basis of their general knowledge, how to determine which clinical or biochemical manifestations are associated with a given disease and which are likely to be improved (treatment) or prevented, delayed or decreased in intensity (prevention). In the context of atopic dermatitis, a clinical parameter of interest may be the gravity of the skin lesions measured by evaluating for example erythema, excoriations, and lichenification, compiled on the composite CADESI score (Olivry T et al., 2014), or quantification of itch. Another clinical parameter may be pruritic behaviour defined as a sequence of scratching, licking, biting, shaking or rubbing of any part of the body (Gonzales et al, 2016).

The following examples aim at illustrating the present invention.

### EXAMPLES

### Example 1: Generation of HEK Blue Stat3 Canine IL31 SEAP Reporter Cell Line

In order to have a convenient and quantitative assay to measure the activation of the canine interleukin 31 receptor (IL-31R consisting of IL-31RA/OSMR subunits), a HEK-Blue™ stable cell line was generated by the biotech company InvivoGen. Human embryonic kidney HEK 293 cell line purchased from ATCC (CRL-1573™) was first stably transfected with the human STAT3 gene, plasmid pUNO1-hSTAT3 (Invivogen Cat. code puno1-hstat3b) to obtain a fully active STAT3 pathway. The other genes of the pathway being naturally expressed in sufficient amounts. These cells were then transfected with a STAT3-inducible secreted embryonic alkaline phosphatase (SEAP) reporter gene (Invivogen Cat. code pNifty ST3-INFb-SEAP). The SEAP reporter gene is under the control of four STAT3 binding sites linked to the IFN-β minimal promoter. Activation of the STAT3 pathway induces the secretion of SEAP The established HEK-Blue STAT3 cells were further transfected with three expression vectors, pSelect2b-dIL31RA, pSelect2b-dOSMR and pSelect-puro-mcs (InvivoGen, Cat. code: psetp-mcs), with a ratio 4-4-1. Upon canine IL-31 stimulation, HEK-Blue™ canine IL-31R cells trigger the activation of STAT3 and the subsequent secretion of SEAP. Levels of STAT3-induced SEAP can be readily monitored using QUANTI-Blue™ solution (InvivoGen Cat. Code rep-qbs). Multiple puromycin resistant clones were grown and selected for sensitivity to canine IL-31.

One of the selected clones was deposited under Budapest treaty at Collection Nationale de Cultures de Microorganismes (CNCM), Pasteur Institute, 25 rue du Dr ROUX, 75724 Paris, Cedex 15, under number 1-5792 on Dec. 8, 2021. This clone was used in the other examples below.

### Example 2: Isolation and Purification of Canine IL31RA Recombinant Protein

Recombinant extra cellular domain of canine IL-31 receptor A (cECD-IL-31RA) was produced and purified 1) to be used for immunization and screening in order to develop antibodies against canine IL31RA and 2) to be used for in vitro assays for characterization of the binding of the antibodies to cECD-IL-31RA (e.g. PK ELISA, potency ELISA, Octet etc.)

### Materials and Methods

A 6 Histidine tag was added at the C-terminal of c-ECD-IL31RA to allow purification by metal ion affinity chromatography (IMAC). Briefly, the cDNA coding for the cECD-IL-31RA (SEQ ID NO: 89) was cloned into pQMCF expression vectors (QMCF technology from Icosagen). Endotoxin free plasmid was transfected into CHOEBNAL T85 1E9 CHO cell line and the established pool of cells was used to produce the recombinant protein in the CHO medium. Transient production was done at 1 L final volume.

SEQ ID NO: 89
VLPAKPENISCIFYYEENFTCTWSPEKEASYTWYKVKRTYSYGYKSDICSTDNSTRGNHASCSFLPPTITNPDNYTI
QVEAQNADGIMKSDITYWNLDAIMKIEPPEIFSVKSVLGIKRMLQIKWIRPVLAPHSSTLKYTLRFRTINSAYWMEVNFTKEDI
DRDETYNLTELQAFTEYVMTLRCAPAESMFWSGWSQEKVGTTEEEAPYGLDLWRVLKPAMVDGRRPVQLMWKKATGAP
VLEKALGYNIWYFPENNTNLTETVNTTNQTHELYLGGKTYWVYVVSYNSLGESPVATLRIPALNEKTFQCIEAMQACLTQD
QLVVEWQSSAPEVDTWMVEWFPDVDSEPSSFSWESVSQARNWTIQKDELKPLWCYNISVYPVLRDRVGQPYSTQAYVQE
GIPSAGPVTQADSIGVKTVTITWKEIPKSKRNGFIKNYTIFYQAEDGKEFSKTVNSNILQYRLESLTRRTSYSLQVMASTNAG
GTNGTKINFKTLSISVLEGGGGSHHHHHHHHHH

On day four after transfection additional feed was added and temperature was shifted from 37° C. to 30° C. for production. Overall protein production time was 12 days. At the end of production, the supernatants were clarified by centrifugation and phenylmethylsulfonyl fluoride (PMSF) was added. The supernatants were frozen and kept at −20° C. until purification.

Recombinant cECD-IL-31RA was then purified by IMAC using HisTrap™ Excel columns (GE Healthcare) followed by preparative gel filtration with Superdex 200 Increase 10/300 GL (GE Healthcare).

### Results

Purified material was analyzed by SDS-PAGE under reducing and non-reducing conditions. In FIG. 2, a broad band spreading between 100 and 140 kd molecular weight markers is clearly visible on the gel indicative of a highly glycosylated protein. The theoretical molecular weight for cECD-IL-31RA is 59 Kd and the protein has fourteen potential N-glycosylation sites which is in accordance with higher and broad band seen on the gel.

### Conclusions

Altogether, from a 1-liter culture, 13 mg of purified cECD-IL-31RA was obtained, 0.22 μm filter sterilized (Merck Millipore), aliquoted and stored at −80° C.

### Example 3: Isolation of Mouse Monoclonal Antibodies that Bind to Canine IL-31RA

Mouse monoclonal antibodies were identified using standard hybridoma technology following immunization of mice with canine IL-31RA extra cellular domain. We used hemizygous Transgenic (Tg) mice that carry five copies of the bovine FcRn α-chain encoding gene (bovine FCGRT) in addition to the endogenous mouse FCGRT gene on BALB/c genetic background [BALB/c_Tg5_Bfcgrt] (Cervenak J, et al. 2011). Wt BALB/c mice were littermates of the Tg animals born from hemizygous breeding. Mice were kept under specified pathogen free (SPF) conditions in individual ventilation cages (IVC) in the animal house at ImmunoGenes Ltd, Budapest, Hungary.

Overexpression of the FcRn in Tg mice extended IgG half-life (Bender B. et al, 2007). These mice also have augmented T dependent humoral immune response, which manifests in higher antigen specific antibody titers, greater number of antigen specific, activated T helper cells, increased number of activated antigen specific B cells, bigger spleen, and increased size and numbers of germinal centers in the spleen after intraperitoneal immunization (Cervenak J, et al. 2011). A larger pool of antigen specific B cells facilitates monoclonal antibody (mAb) discovery as it allows more effective identification of the appropriate B-cell clones either using standard hybridoma or novel high throughput technologies.

### Materials and Methods and Results

FcRn transgenic mice, which are known to have an increased humoral response, were immunized with IL-31RA-ECD-Fc fusion protein.

Two cohorts (6 mice per group) of FcRn transgenic mice were first immunized with 25 ug of IL-31RA-ECD-Fc upon intraperitoneal (IP) administration route and were boosted 3 times every 2 weeks with 12.5 ug of IL-31RA-ECD-Fc upon IP administration route. Serum from immunized mice were screened for binding to IL-31RA-ECD-His tagged by direct ELISA (IL31RA-ECD-His coated on the ELISA plates and serum antibody binding was revealed using an anti-mouse IgG-HRP conjugate). Two mice exhibited a very high antibody titer against IL-31RA-ECD at day 56 post immunization and were selected for hybridoma production. Splenocytes were used for fusion with SP2 myeloma cells in a 2:1 ratio together with peritoneal cells from BalbC mice used as feeder cells.

3000 hybridomas were screened for binding to IL-31RA-ECD-His by direct ELISA. 420 clones were found positive and expanded in 24 well plates. When retested, 46 hybridomas were found to be strongly positive in direct ELISA and were subcloned and cryopreserved. Out of the 46, 28 were confirmed as stable hybridomas after two subsequent subcloning.

These 28 hybridomas were tested for inhibitory activity in the HEK-Blue cellular assay (clone deposited at CNCM on Dec. 8, 2021 under number 1-5792) and only 2 antibodies, 8D3 and 3F1 were found to inhibit canine IL-31 activation potency.

### Conclusions

Two mouse monoclonal antibodies, 8D3 and 3F1 obtained from bFcRn-transgenic mice immunized with canine IL-31RA-ECD were able to inhibit the activation of IL-31RA/OSMR co-receptor by canine IL-31.

### Example 4: Preparation and Characterization of Chimeric Antibodies

Two lead mouse monoclonal antibodies, 8D3 and 3F1 were chosen for cloning into canine IgGB scaffold as chimeric antibodies, to be produced, characterized and their potency in suppressing canine IL-31-induced STAT3 activation tested in the HEK Blue cell-based-assay.

### Materials and Methods

### Chimeric Antibody Preparation

Synthetic codon optimized DNA encoding mouse derived antibody VH and VL sequences were designed, ordered and DNA was cloned using LIC method into the appropriate canine IgGB, kappa pQCMF expression vectors (Icosagen).

In order to produce chimeric antibodies, 15×10⁶ CHOEBNAL T85 1E9 cells were co-transfected with 5 μg of canine light chain and 5 μg of canine heavy chain vector DNAs using Reagent 007 for transient antibody production. The cells were cultivated in 35 ml volume of Xcell CHO-TF medium, for initial 72 h at 37° C., for the production phase, the temperature was shifted to 30° C. and the culture was additionally fed. The duration of the production phase was 9 days. At the end of production phase the cells were removed from expression culture supernatants by centrifugation. Then, clarified supernatants were filtered through the glass fiber prefilter and 0.45 μm filter. Antibodies were purified by MabSelect SuRe affinity chromatography, eluted with 0.1 M Na-citrate pH 3.3 and neutralized with 1.5 M Tris pH 8.8. Collected MabSelect chromatography IgG fractions were concentrated using Amicon Ultra centrifuge filters (Merck Millipore) and gel filtrated with Superdex 200 Increase column into PBS pH 7.4.

Purified antibodies were sterile filtered, concentration was measured with NanoDrop 2000 (Thermo Scientific), aliquoted and stored at −75° C.

Purified antibodies were analyzed under non-reduced (−DTT) and reduced (+DTT) conditions by Coomassie staining on SDS-PAAG. Purity of purified antibodies was analyzed by size-exclusion chromatography (SEC) with Superdex 200 Increase column.

### Cell-Based Assay

For performing cell-based assay the following detailed protocol was used:

Cells of the clone deposited at CNCM on Dec. 8, 2021 under number 1-5792 were grown in DMEM medium containing Penicillin 100 units/mL, Streptomycin 100 μg/mL, Glutamax 1× and 10% heat inactivated FBS.

On day 1, in a 96-well plate, prepare 12 point serial dilution of the antibody to be tested starting at 30 ug/ml with 1/3 dilutions in a final volume of 80 ul. Add 100 μL of cells at a concentration of 0.5×10⁶ cells/mL. Incubate the plate for 1 h at 37° C., 5% CO2. Then add 20 μL of canine IL-31 solution at 0.09 ng/mL. Incubate the plate 24 h at 37° C., 5% CO2.

On day 2, prepare the volume of Quanti-Blue reagent needed to plate 180 μL per well in a new 96-well plate. Add 20 μL of culture supernatant from the overnight cultured 96-well plate and place the plate at 37° C. under agitation (about 130 rpm) for 3 hrs. Measure the absorbance at 640 nm. Perform an IC50 analysis using GraphPad and the [Inhibitor] vs. response—Variable slope (four parameters) model.

### Octet Binding

Binding of the purified antibodies to cECD-IL-31RA was analyzed using the Octet system (BLI technology, Fortebio). Octet K2 instrument was used to measure the binding kinetics of either canine IL-31RA (cIL-31RA) or human IL31RA (hIL-31RA) to 8DE and 3F1 antibodies. cIL-31RA (see example 1) or hIL31R proteins (Acrobiosystem, Cat:ILA-H5256) were attached to the surface of the protein A (fortéBio, Part No: 18-5013) sensor tip (5 ug/ml, 200 ul, 120 sec). Different dilutions (15.6 nM, 3.91 nM, 0.98 nM) were made from the 3F1 and the 8D3 and it was incubated with the cIL-31RA or the hIL31R bound on the tips for 60 sec. Then, the antibodies were allowed to dissociate for 600 seconds. All the dilutions were made in PBS-Tween (0.05%) solution. The measurement was corrected for baseline drift by subtracting a control sensor exposed to running buffer (PBS-Tween, 0.05%) only. Data analysis and curve fitting were carried out using the own software (local fitting, 1:1 Langmuir model) of the device.

### Result

### Octet Binding

The binding measurements of chimeric antibodies 8D3 and 3F1 are summarized in Table 6 below:

TABLE 6
KD values of 8D3 and 3F1 chimeric antibodies measured by Octet.

| | Sample | KD (M) |
|---|---|---|
| 8D3 | cIL31r | <1.0E−12 |
| | hIL31r | No binding |
| 3F1 | cIL31r | <1.0E−12 |
| | hIL31r | No binding |

The Kd value of the 3F1 and IL-31RA interaction was lower than 10E-12 M. Binding between the 3F1 and hIL31RA was not observed with similar concentrations.

The Kd value of the 8D3 and IL-31RA interaction was lower than 10E-12 M. Binding between the 8D3 and hIL31RA was not observed with similar concentrations.

### Cell-Based Assay

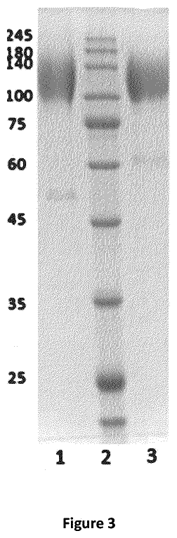

Results are presented in FIG. 3, and show that both 8D3 and 3F1 chimeric antibodies inhibit cIL-31/cIL-31RA signaling pathway, but 8D3 is much more potent than 3F1, despite their similar affinity for cIL-31RA.

### Conclusions

In conclusion, the immunization strategy used permitted to obtain only 2 monoclonal antibodies with high affinity for cIL-31RA, only one of which has high potency for inhibiting the cIL-31/cIL-31RA signaling pathway (8D3 antibody). This antibody was selected for canonization.

### Example 5: Preparation and Characterization of Caninized Versions of 8D3

Antibody caninization was performed using standard CDR grafting method followed by construction and screening of caninized antibody variants containing rationally designed back mutations. Biological activity of the caninized antibodies was determined in cell-based IL31RA dependent STAT3-SEAP reporter assays. The ability to inhibit canine IL-31-induced, IL-31RA mediated STAT3-SEAP reporter activation in HEK-Blue cells was determined by serially diluting each protein-A purified individual antibody and calculating their IC50 concentrations.

In order to guide the caninization of the mouse (Mus musculus) antibody 8D3, we used the yeast display technology. Libraries of single mutants of 8D3 VH and VL where generated and expressed at the surface of the yeast as Fab fragments. A comprehensive way to understand the consequences of mutations within the VH and VL variable regions is to use deep mutational scanning (DMS), which is a technique where each residue of a protein can be mutated to every possible variant (Fowler and Fields, 2014). Each amino acid along the whole VH and VL was substituted with the other 19 natural amino acid. The resulting library of variants is then used in a functional screen (i.e. binding to biotinylated cIL-31RA-ECD by flow cytometry) that simultaneously detects the impact of each mutation through deep sequencing.

One of the challenges of a successful caninization design is the selection of residues from the canine framework to be ‘back-mutated’, i.e. to be substituted with the parental mouse amino acid, in order to prevent loss of affinity or antibody stability due to structural incompatibility of the framework with the engrafted mouse CDR sequences. Typically, these residues are at so-called ‘vernier’ or ‘canonical’ positions. ‘Vernier’ residues are structurally adjacent to the CDRs, and are known to affect CDR conformation and fine-tuning of antigen recognition. ‘Canonical’ residues are positions whose adoption of specific sequence are signatures of cataloged three dimensional conformations of CDR sequences; i.e. an analysis of CDRs in resolved antibody structures permits the classification of these CDR structures on the basis of conformation, and the subsequent association of these conformations with specific sequence signatures at particular sequence locations.

### Materials and Methods

Without being restricted to any specific approach, the overall process of designing caninized versions of anti-canine IL-31 receptor A antibody 8D3 involved the following steps i) identify the amino acid sequence of the VH and VL CDRs of antibody 8D3 as defined by the Kabat nomenclature ii) identify a suitable canine V germline gene for both the VH and the VL that will be used as acceptor sequence iii) Identify the amino acid sequence of the CDRs as defined by the Kabat nomenclature of the canine V germline genes above iv) replace the CDRs in the canine V germline genes with the corresponding VH and VL CDRs of antibody 8D3 v) Replace some canine framework residues with antibody 8D3 framework residues that have been identified as critical by DMS technology vi) Replace some 8D3 CDR residues with mutations that have been identified as beneficial by DMS technology vii) Synthesize the DNA encoding the caninized versions from step (vii), clone it into a suitable expression plasmid, and transfect the plasmids containing desired caninized H and L chains into CHO cells. viii) Purify expressed caninized antibody from CHO supernatant. ix) Test purified caninized antibody for binding to canine IL-31RA by Octet and potency in the HEK-Blue cellular assay using both canine IL-31 for induction. The application of the above outlined steps resulted in a set of caninized VH and VL sequences.

### Results

Parental antibody 8D3 having VH and VL amino acid sequences of SEQ ID NOs: 17 and 18, respectively, were caninized by the introduction of the CDRs as defined by the Kabat nomenclature into the VH of heavy chain frameworks (FR1, FR2 FR3) from the canine germline subgroup IGHV4-1*01 together with canine IGHJ2*01 (FR4), and the introduction into the VL of light chain frameworks (FR1, FR2, FR3) from the canine germline subgroup IGKV4S1*01, together with IGKJ3*01 (FR4).

The germline sequences used for 8D3 caninization efforts resulted in very low expression of the mAbs. Heterochimeras production showed that the chimeric light chain paired with the caninized heavy chain did not express while the caninized light chain paired with the chimeric heavy chain expressed very well. Based on the results obtained from the heterochimeras, it was deduced that the caninized heavy chain was responsible for the loss of expression.

In an effort to restore expression, the caninized heavy chain was modified by introducing 8 substitutions identified by DMS technology in frameworks or CDRs (8D3-VH-B):
LH2V. backmutation Leu to Val at Kabat position H2 in framework1
NH54Q; Asn to Gln at Kabat position H54 in CDR2
FH63L; Phe to Leu at Kabat position H63, Type I Honegger residue
DH67I; Asp to Ile at Kabat position H67, germline mutation
VH82L; Val to Leu at Kabat position H82, germline mutation
IH87T; Ile to Thr at Kabat position H87, germline mutation
SH100A; Ser to Ala at Kabat position H100 in CDR3
CH102S; Cys to Ser at Kabat position H102 in CDR3

(SEQ ID NO: 90)
H2 H54 H63
EVTLQESGPGLVKPSQTLSLTCVASGFSIKDSFIHWLRQRPGRGLEWIGRIDPAQGNTEYDPNLQG
H67 H82 H87 H100 H102
RISITADTAKNQASLQLSSMTTEDTAVYYCARYYYGNAHFDSWGQGTLVTVSS

The caninized antibody resulting from the association of 8D3-VHB with 8D3-VL-A was well expressed but when tested for inhibitory activity in the bioassay was shown to have a much lower activity (around 26 times) than that of the chimeric.

In an effort to restore the potency in the bioassay, 2 extra mutations in CDR3 resulting for the DMS technology were introduced in caninized version 8D3-VHB to create 8D3-VH-G:
NH99A; Asn to Ala at Kabat position 99
AH100S; Ala to Ser at Kabat position 100; backmutation

(SEQ ID NO: 91)
EVTLQESGPGLVKPSQTLSLTCVASGFSIKDSFIHWLRQRPGRGLEWIGRIDPAQGNTEYDPNLQG
H99H100
RISITADTAKNQASLQLSSMTTEDTAVYYCARYYYGASHFDSWGQGTLVTVSS

The caninized antibody resulting from the association of 8D3-VH-G with 8D3-VLA was well expressed and exhibited an inhibitory activity in the bioassay similar to that of the chimeric antibody. The single mutation Asn to Ala at Kabat position 99 in the CDR3 was sufficient to restore the inhibitory activity as assessed in the HEK-Blue bioassay.

Further refinement of the mutations obtained by the DMS technology that were introduced in the caninized versions 8D3-VH-G and 8D2-VL-A identified a series of 14 combinations of caninized VH and VL that were able to express well, have good biophysical properties (e.g. low aggregation propensity, high thermal stability etc.) and have an inhibitory activity similar or better than that of the chimeric antibody.

The 14 caninized versions were produced and purified as described in Example 4 for the chimeric versions. Different combinations of caninized VH and VL versions were produced as described in Table 7 below. All the purified caninized versions were evaluated for their inhibitory potency for canine IL-31 stimulation in the HEK-Blue cell-based SEAP assay following the same protocol as described in Example 4 for the chimeric versions. The IC50 obtained for all caninized versions are shown in Table 7 below. In all individual assays, the chimeric version of antibody 8D3 has been used as the reference molecule.

TABLE 7
Specific cell productivity (pg/cell) and mean IC50 (nM) of 14 caninized
versions of 8D3 antibody compared to chimeric 8D3 antibody.

| | Specific cell | Mean |
|---|---|---|
| | productivity | IC50 (nM) |
| | (pg/cell) | (n = 3) |
| 8D3 (chimeric) | 21.5 | 0.43 |
| b) 8D3-clone7v2-VH/8D3-clone7-VL | 53.4 | 0.44 |
| h) 8D3-VH-H/8D3-VL-Ev2 | 48.5 | 0.59 |
| c) 8D3- VL-L/8D3-VL-Ev2 | 48.6 | 0.41 |
| d) 8D3- VH-L/8D3-VL-G | 51.4 | 0.44 |
| a1) 8D3-VH-L/8D3-VL-H | 48.1 | 0.33 |
| (VTQ2101) | | |
| i) 8D3-VH-Lv2/8D3-VL-E | 40.9 | 0.49 |
| e) 8D3-VH-Lv2/8D3-VL-Ev2 | 40.3 | 0.38 |
| f) 8D3-VH-Lv2/8D3-VL-G | 33.5 | 0.41 |
| j) 8D3-VH-Lv2/8D3-VL-H | 43.9 | 0.54 |
| g) 8D3-VH-N/8D3-VL-E | 41.9 | 0.44 |
| k) 8D3-VH-N/8D3-VLEv2 | 41 | 0.54 |
| l) 8D3-VH-518/8D3-VL-A | 44.6 | 0.54 |
| m) 8D3-VH-518/8D3-VL-Ev2 | 59.5 | 0.57 |
| n) 8D3-VH-518H/8D3-VL-A | 35.7 | 0.48 |

### Conclusions

Starting with variable VH and VL sequences from murine origin, it was possible after different engineering steps and especially introduction of mutations in the FR regions and CDRs to obtain several caninized versions (VHL/VLH) of 8D3 antibody with good specific cell productivity and a higher or similar inhibitory potency in the cell-based assay than that of the chimeric version.

### Example 6: Comparison with Prior Art Anti-Canine IL-31 and Anti-Canine IL-31RA Antibodies

The objective of this study was to compare the inhibitory potency for canine IL-31 stimulation in the HEK-Blue cell-based SEAP assay of VTQ2101 (8D3-4-1-VH-L/8D3-451-VL-H, see Example 5 above) to prior art anti-IL31 pathway candidates:
Cytopoint®, a canine anti-IL31 (Cas number: 1533403-95-0)
Tirnovetmab, a canine anti-IL31 (Cas number: 2364504-80-1)
Nemolizumab, a human anti-hIL31RA (Cas number: 1476039-58-3)
10H12 chimeric, 10H12 caninized, 51G4, 27A10, 44E2, 4G7, 28E12, 53B3, anti-cIL31RA candidates described in provisional applications U.S. 63/092,294 and U.S. 63/092,296 available in the history file of application WO2021/123094 which claims the priority thereof.

### Materials and Methods

VTQ2101 and all the candidates were evaluated for their inhibitory potency for canine IL-31 stimulation in the HEK-Blue cell-based SEAP assay (clone deposited at CNCM on Dec. 8, 2021 under number 1-5792) following the same protocol as described in Example 4 for the chimeric versions.

### Results

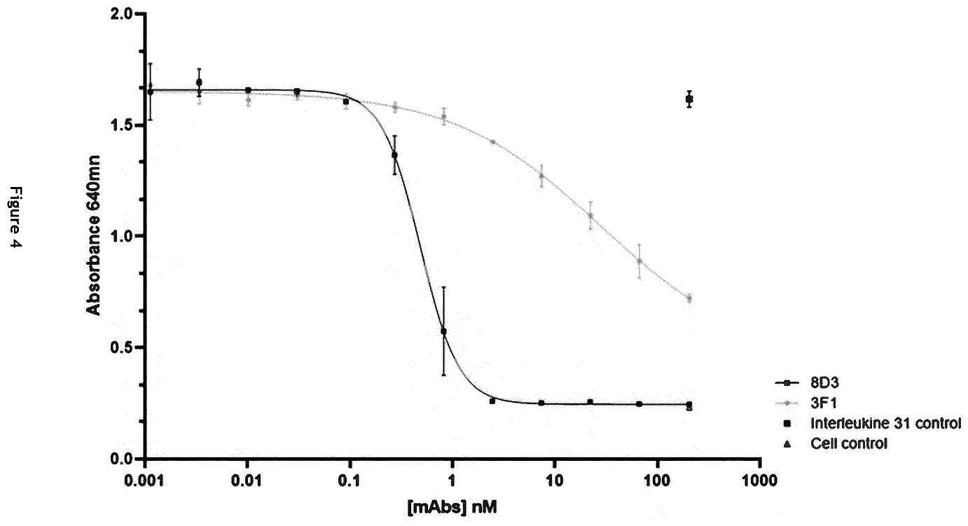

The IC50 obtained for all candidates are depicted in FIGS. 4, 5 and 6. IC50 from FIG. 5 are shown in Table 8 below with IC50 ratio versus VTQ2101.

TABLE 8
IC50 values in the HEK-Blue cell-based SEAP assay based
on curves of FIG. 5 and IC50 ratio versus VTQ2101.

| | | | | | | IC50 ratio (IC50 |
|---|---|---|---|---|---|---|
| | | | | Ecart | | candidate/IC50 |
| | N = 1 | N = 2 | Moyenne | type | CV | VTQ2101 |
| 10H12 | 670.485 | 1355.238 | 1012.862 | 484.19 | 48% | 3492 |
| 51G4 | 3.15E+10 | 4.15E+10 | 3.65E+10 | 7.01E+09 | 19% | 16.4 E+10 |
| 27A10 | 1.19E+10 | 1.40E+10 | 1.29E+10 | 1.49E+09 | 12% | 6.2 E+10 |
| 44E2 | 6.66E+10 | 2.20E+07 | 3.33E+10 | 4.71E+10 | 141% | 34.7 E+10 |
| 4G7 | 4.24E+09 | 1.39E+08 | 2.19E+09 | 2.90E+09 | 132% | 22.1 E+09 |
| 28F12 | 1.082 | 1.470 | 1.276 | 0.274 | 22% | 5.6 |
| 53B3 | 10.478 | 21.091 | 15.785 | 7.505 | 48% | 54.6 |
| VTQ2101 | 0.192 | 0.213 | 0.203 | 0.015 | 8% | 1 |

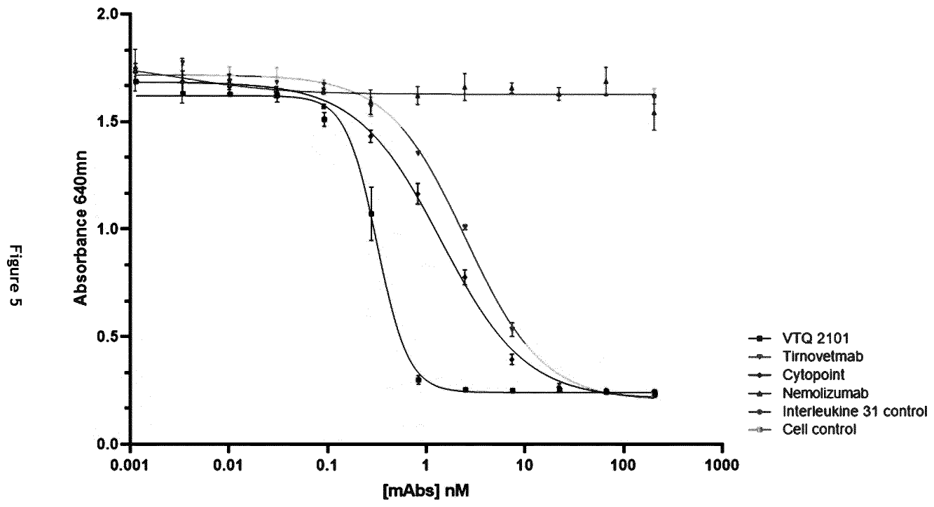

The results show that VTQ2101 has an IC50 at least 5-fold lower than a monoclonal anti-cIL-31RA antibody 28F12 which is the best anti-cIL31RA candidate in the prior art (see FIG. 5 and Table 8 above). It should also be noted that the only caninized version of antibody 28F12 disclosed in provisional applications U.S. 63/092,294 and U.S. 63/092,296 available in the history file of application WO2021/123094 which claims the priority thereof has much lower potency (i.e. much higher IC50 than its chimeric version (see FIG. 6).

The study also shows that VTQ2101 has a lower IC50 than Cytopoint® and Tirnovetmab and that Nemolizumab has no inhibitory potency for canine 1L31/cIL31RA pathway (see FIG. 4).

### Example 7: In Vivo Experiments

### Materials and Methods

This study was based on the il-31 model study described in the Gonzales et al. 2016 publication.

The objective of this study was to compare the efficacy of 8D3, a caninised anti-cIL31RA monoclonal antibody, to Cytopoint® at the same dose (1 mg/kg) to control IL31 induced pruritus in dogs.

It was a negative and positive controlled, randomised, masked, parallel study design with three treatment groups:
Group 1 (3 dogs): untreated
Group 2 (6 dogs): Cytopoint®, single SC dose at 1 mg/kg
Group 3 (6 dogs): 8D3 VH-L/VL-H anti-cIL31R mAb (VTQ 2101), single SC dose at 1 mg/kg

Treatments were administered to dogs in groups 2 and 3 on study day 0 (=DO).

Before treatment administration, on study day D-7, post-IL31 pruritus scores were established for all included dogs. The dogs were randomised to the respective treatment groups based on the post-IL31 pruritus scores to ensure a homogeneity of pruritus scores between groups.

The pruritus scores were established on study days (D-7, D7, D14, D28, D42, D58 and D77). Technicians observing the dogs in real time were masked to the treatment groups, each technician observed 3 dogs. On days of pruritus evaluation, the dogs were placed into individual pens and after one hour of acclimatization, baseline pruritus (i.e. before IL31 injection) was determined over a 30 minute observation period. At the end of this first observation sequence, canine IL31 (1.75 μg/kg) was administered intravenously and 30 minutes thereafter, the dogs were observed for two hours to record the post-IL31 induced pruritus. The pruritus score corresponded to the number of minutes (over 2 hours) where at least one pruritic behaviour was observed. Pruritic behaviour was defined as a sequence of scratching, licking, biting, shaking or rubbing of any part of the body.

Following treatment administrations, the pruritic score post-IL31 challenge increased gradually from week 4 (D28) in the Cytopoint® treated group and from week 8 (D58) in the VTQ2101 treated group.

### Results

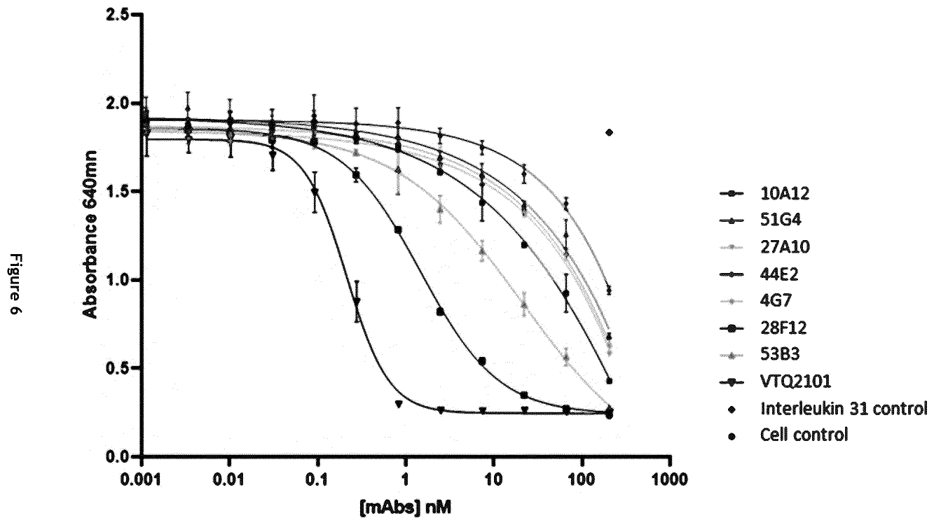

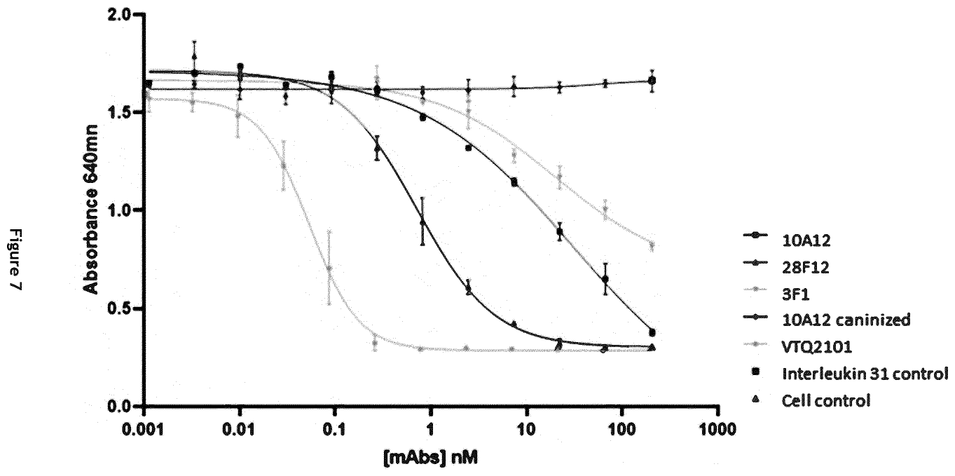

Results of pruritic score on average (baseline and post IL-31) for the three groups are summarized in Table 9 below and depicted in FIG. 7.

TABLE 9
Results of average baseline and average post IL-31 pruritic scores
for group 1 (untreated), group 2 (Cytopoint ®, single SC
dose at 1 mg/kg) and group 3 (VTQ 2101, single SC dose at 1 mg/kg).

| | | D −7 | D 7 | D 14 | D 28 | D 42 | D 58 | D 77 |
|---|---|---|---|---|---|---|---|---|
| Average | Group 1 | 2.3 | 0.7 | 2 | 1.7 | 2.3 | 1.5 | 2.3 |
| Baseline | Group 2 | 1.2 | 6.2 | 4.7 | 6.5 | 3.2 | 6.7 | 7 |
| | Group 3 | 1 | 3 | 5 | 2.3 | 2 | 5.6 | 5 |
| Average | Group 1 | 89 | 83.7 | 78 | 92.7 | 95 | 90.5 | 79.5 |
| Post-Il31 | Group 2 | 87.5 | 7.3 | 10.3 | 16.3 | 42 | 64.2 | 75.2 |
| | Group 3 | 91 | 7.3 | 5.3 | 19.3 | 32.2 | 29.8 | 72.2 |

Similar results were obtained in another experiment where group 3 was treated with chimeric 8D3 antibody (data not shown).

### Conclusions

The results of this study show that a single SC administration of VTQ2101, a caninized monoclonal IL31R antibody at a dose of 1 mg/kg, was able to inhibit pruritus in dogs for 8 weeks (D58), in this canine IL31-induced itch model. The study also shows that anti-IL31RA VTQ2101 is effective at least 16 days longer for treating atopic dermatitis than anti-cIL31 antibody Cytopoint®.

### Example 8: Antigen-Binding Derivatives

The following antigen-binding derivatives of the anti-canine IL-31RA antibody according to the invention were generated.

Format 1. scFv-Fc-VTQ2201 (scFv-VTQ2201-HLλcap-GS18-Mouse IgG2A-LALAPG-Fc)

This antigen-binding derivative is a single chain Fv (scFv) format of the original anti-canine IL-31RA mouse antibody 8D3 fused with a mouse IgG2a Fc containing the mutations L234A, L235A and P329G.

The scFv was designed by fusing the variable region of the heavy chain (VH) to the variable of the light chain (VL) using an 18-long peptide linker (GS18) having the following sequence GSTSGGGSGGGSGGGGSS (SEQ ID NO: 95). In the VL, the C-terminal region corresponding to the J gene which was a Kappa J gene in the original mouse 8D3 was replaced by a Lambda J gene (FGSGTKLTVLG; SEQ ID NO: 104). The Fc is made of the hinge region, CH2 and CH3 domains of a mouse IgG2a where 3 mutations (L234A, L235A and P329G) where introduced in order decrease Fc-related effector functions.

The complete sequence of this antigen-binding derivative is the following:

(SEQ ID NO: 97)
EVQLQQSGAELVKPGASVKLSCTASGFNIKDSFIHWLKQRPEQGLEWIGRIDPANGNTEYDPNFQGKVTITADT
SSNTAYLQLSSLTSEDTAVYYCARYYYGNSHFDAWGQGTTLTVSSGSTSGGGSGGGSGGGGSSRIVMNQTPK
FLPISAGDRVIITCKASQSVTNDVTWYQQKPGQSPKVLIHYASNRYTGVPDRFTGSGYGTDFTFTISTVQAEDLA
VYFCQQDYSSPFTFGSGTKLTVLGPRGPTIKPCPPCKCPAPNAAGGPSVFIFPPKIKDVLMISLSPIVTCVVVDVS
EDDPDVQISWFVNNVEVHTAQTQTHREDYNSTLRVVSALPIQHQDWMSGKEFKCKVNNKDLGAPIERTISKPK
GSVRAPQVYVLPPPEEEMTKKQVTLTCMVTDFMPEDIYVEWTNNGKTELNYKNTEPVLDSDGSYFMYSKLRVE
KKNWVERNSYSCSVVHEGLHNHHTTKSFSRTPGK.

An advantageous antigen-binding derivative is also the following: EVQLQQSGAELVKPGASVKLSCTASGFNIKDSFIHWLKQRPEQGLEWIGRIDPANGNTEYDPNFQGKVTITADT SSNTAYLQLSSLTSEDTAVYYCARYYYGNSHFDAWGQGTTLTVSSGSTSGGGSGGGSGGGGSSRIVMNQTPK FLPISAGDRVIITCKASQSVTNDVTWYQQKPGQSPKVLIHYASNRYTGVPDRFTGSGYGTDFTFTISTVQAEDLA VYFCQQDYSSPFTFGSGTKLTVLG (SEQ ID NO: 106), with any Fc or preferably canine Fc.

Format 2. scFv-Fc-VTQ2202 (scFv-VTQ2202-HLλcap-GS18-MOUSE IgG2A-LALAPG-Fc)

This antigen-binding derivative is a single chain Fv (scFv) format of the original anti-canine IL-31RA mouse antibody 8D3 where all six CDRs have been swapped with the CDRs of lead caninized version of mouse 8D3; VH-L/VL-H and fused with a mouse IgG2a Fc containing the mutations L234A, L235A and P329G.

The scFv was designed by fusing the variable region of the heavy chain (VH) to the variable of the light chain (VL) using an 18-long peptide linker (GS18) having the following sequence GSTSGGGSGGGSGGGGSS (SEQ ID NO: 95). In the VL, the C-terminal region corresponding to the J gene which was a Kappa J gene in the original mouse 8D3 was replaced by a Lambda J gene (FGSGTKLTVLG; SEQ ID NO: 104). The Fc is made of the hinge region, CH2 and CH3 domains of a mouse IgG2a where 3 mutations (L234A, L235A and P329G) where introduced in order decrease Fc-related effector functions.

The complete sequence of this antigen-binding derivative is the following:

(SEQ ID NO: 98)
EVQLQQSGAELVKPGASVKLSCTASGFNIKSSFIHWLKQRPEQGLEWIGRIDPAFGATEYNPAFQGKVTITADT
SSNTAYLQLSSLTSEDTAVYYCARYHYAASHFDAWGQGTTLTVSSGSTSGGGSGGGSGGGGSSRIVMNQTPKF
LPISAGDRVIITCKSSQSVTNDLTWYQQKPGQSPKVLIHYASQRYTGVPDRFTGSGYGTDFTFTISTVQAEDLAV
YFCQQDYASPFTFGSGTKLTVLGPRGPTIKPCPPCKCPAPNAAGGPSVFIFPPKIKDVLMISLSPIVTCVVVDVSE
DDPDVQISWFVNNVEVHTAQTQTHREDYNSTLRVVSALPIQHQDWMSGKEFKCKVNNKDLGAPIERTISKPKG
SVRAPQVYVLPPPEEEMTKKQVTLTCMVTDFMPEDIYVEWTNNGKTELNYKNTEPVLDSDGSYFMYSKLRVEK
KNWVERNSYSCSVVHEGLHNHHTTKSFSRTPGK.

An advantageous antigen-binding derivative is also the following: EVQLQQSGAELVKPGASVKLSCTASGFNIKSSFIHWLKQRPEQGLEWIGRIDPAFGATEYNPAFQGKVTITADT SSNTAYLQLSSLTSEDTAVYYCARYHYAASHFDAWGQGTTLTVSSGSTSGGGSGGGSGGGGSSRIVMNQTPKF LPISAGDRVIITCKSSQSVTNDLTWYQQKPGQSPKVLIHYASQRYTGVPDRFTGSGYGTDFTFTISTVQAEDLAV YFCQQDYASPFTFGSGTKLTVLG (SEQ ID NO: 107), with any Fc or preferably canine Fc.

Format 3. scFv-HLλcap-Fc-VTQ2102 (scFv-VTQ2102-HLλcap-GS18-Canine IgG2B-LALAPG-Fc

This antigen-binding derivative is a single chain Fv (scFv) format of the caninized version VH-L/VL-H of the anti-canine IL-31RA mouse antibody 8D3 fused with a canine IgG2B Fc containing the mutations M234A, L235A and P329G.

The scFv was designed by fusing the variable region of the heavy chain (VH) to the variable of the light chain (VL) using an 18-long peptide linker (GS18) having the following sequence GSTSGGGSGGGSGGGGSS (SEQ ID NO: 95). In the VL, the C-terminal region corresponding to the J gene which was a Kappa J gene in VLH was replaced by a Lambda J gene (FGGGTKLTVLG; SEQ ID NO: 105). The Fc is made of the hinge region, CH2 and CH3 domains of a canine IgG2B where 3 mutations (M234A, L235A and P329G) where introduced in order to decrease Fc-related effector functions.

The complete sequence of this antigen-binding derivative is the following:

(SEQ ID NO: 99)
EVTLQESGPGLVKPSQTLSLTCVASGFSIKSSFIHWLRQRPGRGLEWI
GRIDPAFGATEYNPAFQGRFSITADTAKNQASLQLSSMTTEDSAVYYC
ARYHYAASHFDAWGQGTLVTVSSGSTSGGGSGGGSGGGGSSRIVMTQS
PGSLAGSVGESVSINCKSSQSVTNDLTWYQQKPGEAPKVLITYASQRY
TGVPARFSGSGYGTDFTLTINNLQAEDVGDYFCQQDYASPFTFGGGTK
LTVLGPKRENGRVPRPPDCPKCPAPEAAGGPSVFIFPPKPKDTLLIAR
TPEVTCVVVDLDPEDPEVQISWFVDGKQMQTAKTQPREEQFNGTYRVV
SVLPIGHQDWLKGKQFTCKVNNKALGSPIERTISKARGQAHQPSVYVL
PPSREELSKNTVSLTCLIKDFFPPDIDVEWQSNGQQEPESKYRTTPPQ
LDEDGSYFLYSKLSVDKSRWQRGDTFICAVMHEALHNHYTQESLSHSP
G.

An advantageous antigen-binding derivative is also the following: EVTLQESGPGLVKPSQTLSLTCVASGFSIKSSFIHWLRQRPGRGLEWIGRIDPAFGATEYNPAFQGRFSITADTA KNQASLQLSSMTTEDSAVYYCARYHYAASHFDAWGQGTLVTVSSGSTSGGGSGGGSGGGGSSRIVMTQSPGS LAGSVGESVSINCKSSQSVTNDLTWYQQKPGEAPKVLITYASQRYTGVPARFSGSGYGTDFTLTINNLQAEDVG DYFCQQDYASPFTFGG GTKLTVLG (SEQ ID NO: 108), with any Fc or preferably canine Fc.

Format 4. scFv-HL-Fc-VTQ2102 (scFv-VTQ2102-HL-GS18-Canine IgG2B-LALAPG-Fc)

This antigen-binding derivative is a single chain Fv (scFv) format of the caninized version VH-L/VL-H of the anti-canine IL-31RA mouse antibody 8D3 fused with a canine IgG2B Fc containing the mutations M234A, L235A and P329G.

The scFv was designed by fusing the variable region of the heavy chain (VH) to the variable of the light chain (VL) using an 18-long peptide linker (GS18) having the following sequence GSTSGGGSGGGSGGGGSS (SEQ ID NO: 95). The Fc is made of the hinge region, CH2 and CH3 domains of a canine IgG2B where 3 mutations (M234A, L235A and P329G) where introduced in order to decrease Fc-related effector functions.

The complete sequence of this antigen-binding derivative is the following:

(SEQ ID NO: 100)
EVTLQESGPGLVKPSQTLSLTCVASGFSIKSSFIHWLRQRPGRGLEWI
GRIDPAFGATEYNPAFQGRFSITADTAKNQASLQLSSMTTEDSAVYYC
ARYHYAASHFDAWGQGTLVTVSSGSTSGGGSGGGSGGGGSSRIVMTQS
PGSLAGSVGESVSINCKSSQSVTNDLTWYQQKPGEAPKVLITYASQRY
TGVPARFSGSGYGTDFTLTINNLQAEDVGDYFCQQDYASPFTFGQGTK
LEIKPKRENGRVPRPPDCPKCPAPEAAGGPSVFIFPPKPKDTLLIART
PEVTCVVVDLDPEDPEVQISWFVDGKQMQTAKTQPREEQFNGTYRVVS
VLPIGHQDWLKGKQFTCKVNNKALGSPIERTISKARGQAHQPSVYVLP
PSREELSKNTVSLTCLIKDFFPPDIDVEWQSNGQQEPESKYRTTPPQL
DEDGSYFLYSKLSVDKSRWQRGDTFICAVMHEALHNHYTQESLSHSPG.

An advantageous antigen-binding derivative is also the following: EVTLQESGPGLVKPSQTLSLTCVASGFSIKSSFIHWLRQRPGRGLEWIGRIDPAFGATEYNPAFQGRFSITADTA KNQASLQLSSMTTEDSAVYYCARYHYAASHFDAWGQGTLVTVSSGSTSGGGSGGGSGGGGSSRIVMTQSPGS LAGSVGESVSINCKSSQSVTNDLTWYQQKPGEAPKVLITYASQRYTGVPARFSGSGYGTDFTLTINNLQAEDVG DYFCQQDYASPFTFGQGTKLEIK (SEQ ID NO: 109), with any Fc or preferably canine Fc.

Format 5. scFab-Fc-VTQ2101(scFab-VTQ2101-HLλcap-GS18-Canine IgG2B-LALAPG-Fc)

This antigen-binding derivative is a single chain Fab (scFab) format of the caninized version VH-L/VL-H of the anti-canine IL-31RA mouse antibody 8D3 fused with a canine IgG2B Fc containing the mutations M234A, L235A and P329G.

The scFab was designed by fusing the light chain (VL-CKappa) to the heavy chain (VH-CH1-CH2-CH3) using a 50-long peptide linker having the following sequence GSSGSGSGSTGTSSSGTGTSAGTTGTSASTSGSGSGGGGGSGGGGSAGG (SEQ ID NO: 96). Three (3) mutations were introduced in the Fc (M234A, L235A and P329G) to decrease Fc-related effector functions.

The complete sequence of this antigen-binding derivative is the following:

(SEQ ID NO: 101)
RIVMTQSPGSLAGSVGESVSINCKSSQSVTNDLTWYQQKPGEAPKVLI
TYASQRYTGVPARFSGSGYGTDFTLTINNLQAEDVGDYFCQQDYASPF
TFGQGTKLEIKRNDAQPAVYLFQPSPDQLHTGSASVVCLLNSFYPKDI
NVKWKVDGVIQDTGIQESVTEQDKDSTYSLSSTLTMSSTEYLSHELYS
CEITHKSLPSTLIKSFQRSECGGSSGSGSGSTGTSSSGTGTSAGTTGT
SASTSGSGSGGGGGSGGGGSAGGEVTLQESGPGLVKPSQTLSLTCVAS
GFSIKSSFIHWLRQRPGRGLEWIGRIDPAFGATEYNPAFQGRFSITAD
TAKNQASLQLSSMTTEDSAVYYCARYHYAASHFDAWGQGTLVTVSSAS
TTAPSVFPLAPSCGSTSGSTVALACLVSGYFPEPVTVSWNSGSLTSGV
HTFPSVLQSSGLYSLSSMVTVPSSRWPSETFTCNVAHPASKTKVDKPV
PKRENGRVPRPPDCPKCPAPEAAGGPSVFIFPPKPKDTLLIARTPEVT
CVVVDLDPEDPEVQISWFVDGKQMQTAKTQPREEQFNGTYRVVSVLPI
GHQDWLKGKQFTCKVNNKALGSPIERTISKARGQAHQPSVYVLPPSRE
ELSKNTVSLTCLIKDFFPPDIDVEWQSNGQQEPESKYRTTPPQLDEDG
SYFLYSKLSVDKSRWQRGDTFICAVMHEALHNHYTQESLSHSPG.

An advantageous antigen-binding derivative is also the following: RIVMTQSPGSLAGSVGESVSINCKSSQSVTNDLTWYQQKPGEAPKVLITYASQRYTGVPARFSGSGYGTDFTLTI NNLQAEDVGDYFCQQDYASPFTFGQGTKLEIKRNDAQPAVYLFQPSPDQLHTGSASVVCLLNSFYPKDINVKW KVDGVIQDTGIQESVTEQDKDSTYSLSSTLTMSSTEYLSHELYSCEITHKSLPSTLIKSFQRSECGGSSGSGSGSTG TSSSGTGTSAGTTGTSASTSGSGSGGGGGSGGGGSAGGEVTLQESGPGLVKPSQTLSLTCVASGFSIKSSFIH WLRQRPGRGLEWIGRIDPAFGATEYNPAFQGRFSITADTAKNQASLQLSSMTTEDSAVYYCARYHYAASHFDA WGQGTLVTVSSASTTAPSVFPLAPSCGSTSGSTVALACLVSGYFPEPVTVSWNSGSLTSGVHTFPSVLQSSGLY SLSSMVTVPSSRWPSETFTCNVAHPASKTKVDKPV (SEQ ID NO: 110), with any Fc or preferably canine Fc.

Format 6. scFv-VTQ2101-LH-Fc (scFv-VTQ2101-LH-GS18-Canine IgG2B-LALAPG-Fc)

This antigen-binding derivative is a single chain Fv (scFv) format of the caninized VH-L/VL-H anti-canine IL-31RA mouse antibody 8D3 fused with a canine IgG2B Fc containing the mutations M234A, L235A and P329G.

The scFv was designed by fusing the variable region of the light chain (VL-H) to the variable of the heavy chain (VH-L) using an 18-long peptide linker (GS18) having the following sequence GSTSGGGSGGGSGGGGSS (SEQ ID NO: 95). The Fc is made of the hinge region, CH2 and CH3 domains of a canine IgG2B where 3 mutations (M234A, L235A and P329G) where introduced in order decrease Fc-related effector functions.

The complete sequence of this antigen-binding derivative is the following:

(SEQ ID NO: 102)
RIVMTQSPGSLAGSVGESVSINCKSSQSVTNDLTWYQQKPGEAPKVLI
TYASQRYTGVPARFSGSGYGTDFTLTINNLQAEDVGDYFCQQDYASPF
TFGQGTKLEIKGSTSGGGSGGGSGGGGSSEVTLQESGPGLVKPSQTLS
LTCVASGFSIKSSFIHWLRQRPGRGLEWIGRIDPAFGATEYNPAFQGR
FSITADTAKNQASLQLSSMTTEDSAVYYCARYHYAASHFDAWGQGTLV
TVSSPKRENGRVPRPPDCPKCPAPEAAGGPSVFIFPPKPKDTLLIART
PEVTCVVVDLDPEDPEVQISWFVDGKQMQTAKTQPREEQFNGTYRVVS
VLPIGHQDWLKGKQFTCKVNNKALGSPIERTISKARGQAHQPSVYVLP
PSREELSKNTVSLTCLIKDFFPPDIDVEWQSNGQQEPESKYRTTPPQL
DEDGSYFLYSKLSVDKSRWQRGDTFICAVMHEALHNHYTQESLSHSPG.

An advantageous antigen-binding derivative is also the following: RIVMTQSPGSLAGSVGESVSINCKSSQSVTNDLTWYQQKPGEAPKVLITYASQRYTGVPARFSGSGYGTDFTLTI NNLQAEDVGDYFCQQDYASPFTFGQGTKLEIKGSTSGGGSGGGSGGGGSSEVTLQESGPGLVKPSQTLSLTC VASGFSIKSSFIHWLRQRPGRGLEWIGRIDPAFGATEYNPAFQGRFSITADTAKNQASLQLSSMTTEDSAVYYCA RYHYAASHFDAWGQGTLVTVSS (SEQ ID NO: 111), with any Fc or preferably canine Fc.

Format 7. scFv-VTQ2101-LHλcap-Fc (scFv-VTQ2101-LHλcap-GS18-Canine IgG2B-LALAPG-Fc)

This antigen-binding derivative is a single chain Fv (scFv) format of the caninized VH-L/VL-H anti-canine IL-31RA mouse antibody 8D3 fused with a canine IgG2B Fc containing the mutations M234A, L235A and P329G.

The scFv was designed by fusing the variable region of the light chain (VL-H) to the variable of the heavy chain (VH-L) using an 18-long peptide linker (GS18) having the following sequence GSTSGGGSGGGSGGGGSS (SEQ ID NO: 95). In the VL, the C-terminal region corresponding to the J gene which was a Kappa J gene in VLH was replaced by a Lambda J gene (FGGGTKLTVLG; SEQ ID NO: 105). The Fc is made of the hinge region, CH2 and CH3 domains of a canine IgG2B where 3 mutations (L234AM234A, L235A and P329G) where introduced in order decrease Fc-related effector functions.

The complete sequence of this antigen-binding derivative is the following:

(SEQ ID NO: 103)
RIVMTQSPGSLAGSVGESVSINCKSSQSVTNDLTWYQQKPGEAPKVLI
TYASQRYTGVPARFSGSGYGTDFTLTINNLQAEDVGDYFCQQDYASPF
TFGGGTKLTVLGGSTSGGGSGGGSGGGGSSEVTLQESGPGLVKPSQTL
SLTCVASGFSIKSSFIHWLRQRPGRGLEWIGRIDPAFGATEYNPAFQG
RFSITADTAKNQASLQLSSMTTEDSAVYYCARYHYAASHFDAWGQGTL
VTVSSPKRENGRVPRPPDCPKCPAPEAAGGPSVFIFPPKPKDTLLIAR
TPEVTCVVVDLDPEDPEVQISWFVDGKQMQTAKTQPREEQFNGTYRVV
SVLPIGHQDWLKGKQFTCKVNNKALGSPIERTISKARGQAHQPSVYVL
PPSREELSKNTVSLTCLIKDFFPPDIDVEWQSNGQQEPESKYRTTPPQ
LDEDGSYFLYSKLSVDKSRWQRGDTFICAVMHEALHNHYTQESLSHSP
G.

An advantageous antigen-binding derivative is also the following:

An advantageous antigen-binding derivative is also the following: RIVMTQSPGSLAGSVGESVSINCKSSQSVTNDLTWYQQKPGEAPKVLITYASQRYTGVPARFSGSGYGTDFTLTI NNLQAEDVGDYFCQQDYASPFTFGGGTKLTVLGGSTSGGGSGGGSGGGGSSEVTLQESGPGLVKPSQTLSLT CVASGFSIKSSFIHWLRQRPGRGLEWIGRIDPAFGATEYNPAFQGRFSITADTAKNQASLQLSSMTTEDSAVYYC ARYHYAASHFDAWGQGTLVTVSS (SEQ ID NO: 112), with any Fc or preferably canine Fc.

### Example 9: Inhibitory Potency of Antigen-Binding Derivatives

The objective was to compare the inhibitory potency for canine IL-31 stimulation in the HEK-Blue cell-based SEAP assay of VTQ2101 (8D3-4-1-VH-L/8D3-451-VL-H, see Example 5 above) to its variants (different formats antigen-binding derivatives:
scFv-Fc-VTQ2201 (FORMAT 1.)
scFv-Fc-VTQ2202 (FORMAT 2.)

### Materials and Methods

VTQ2101 and all the candidates were evaluated for their inhibitory potency for canine IL-31 stimulation in the HEK-Blue cell-based SEAP assay (clone deposited at CNCM on Dec. 8, 2021 under number 1-5792) following the same protocol as described in Example 4 for the chimeric versions.

### Results

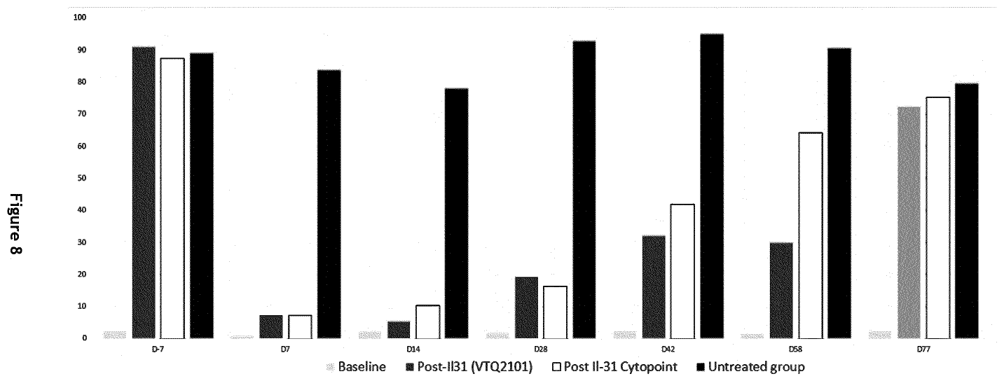

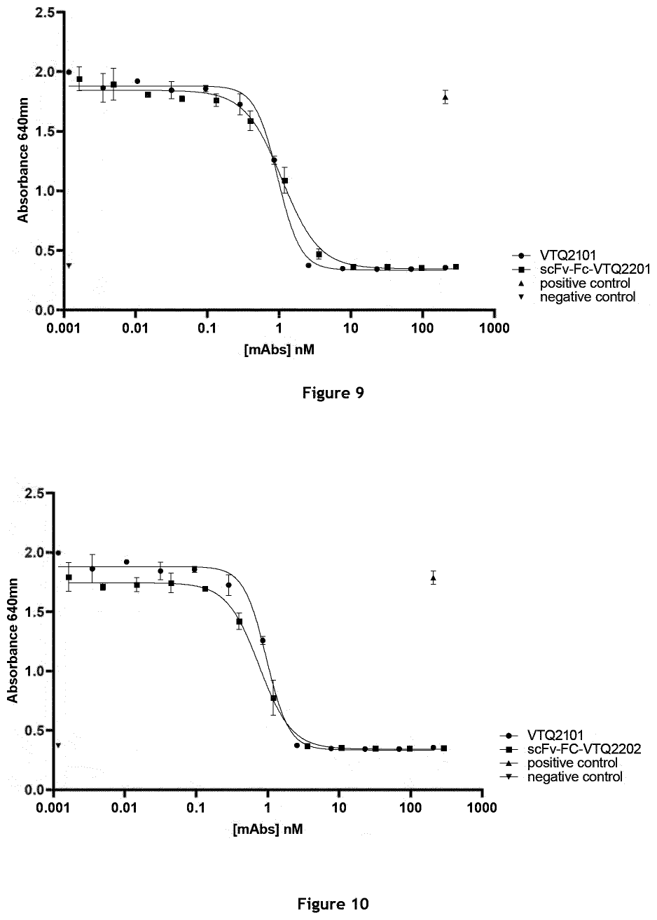

The IC50 obtained for all candidates are depicted in FIGS. 9 and 10 and IC50 ratios versus VTQ2101 are summarized in Table 10 below.

| | | IC50 ratio (IC50 candidate/ |
|---|---|---|
| | FORMAT | IC50 VTQ2101) |
| VTQ2101 (LEAD) | | 1 |
| scFv-Fc-VTQ2201 | 1 | 1 |
| scFv-Fc-VTQ2202 | 2 | 1 |

Similarly, the inhibitory potency for canine IL-31 stimulation in the HEK-Blue cell-based SEAP assay of VTQ2101 (8D3-4-1-VH-L/8D3-4S1-VL-H, see Example 5 above) was also compared to the following other variants (different formats antigen-binding derivatives):
ScFv-VTQ2101-LH-GS18-FC (FORMAT 6.)
ScFv-VTQ2101-LHλcap-GS18-FC (FORMAT 7.)
ScFv-VTQ2102-HLλcap-GS18-FC (FORMAT 3.)
scFab-VTQ2101-FC (FORMAT 5.)

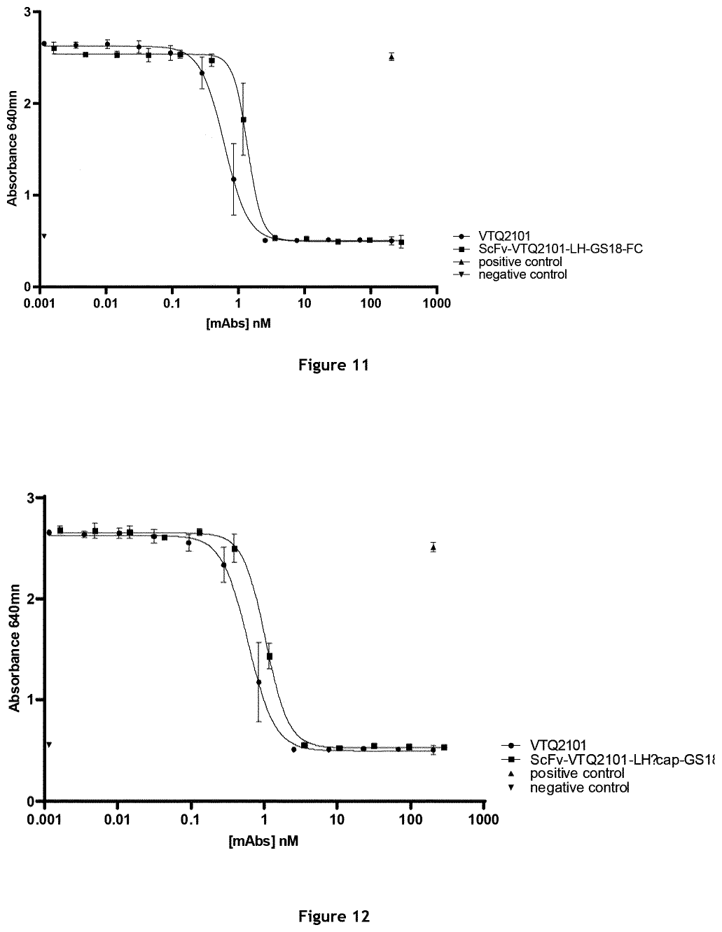

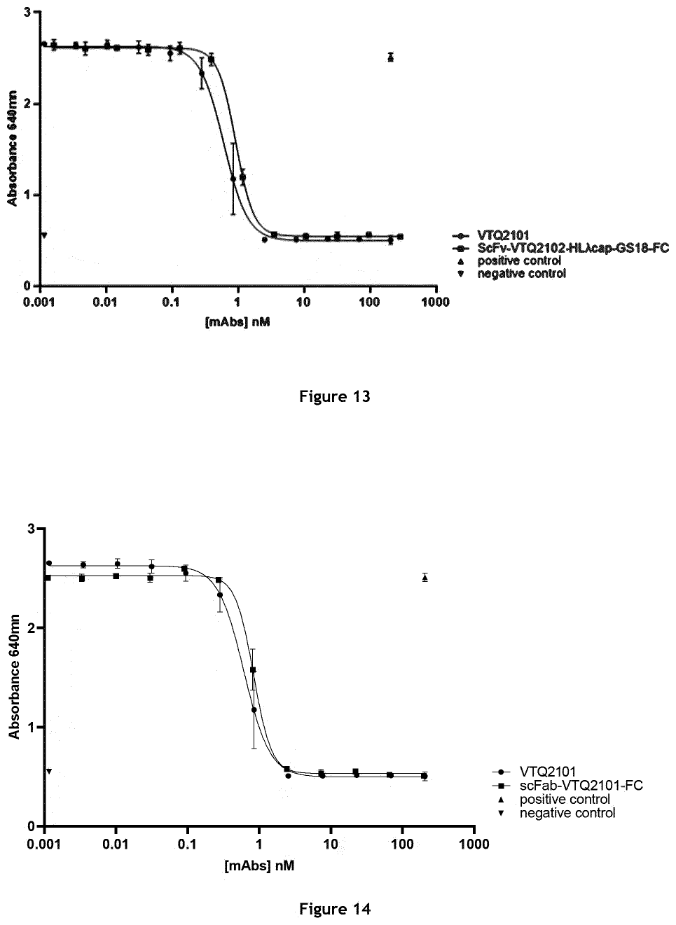

The IC50 obtained for all these candidates are depicted in FIGS. 11-14 and IC50 ratios versus VTQ2101 are summarized in Table 11 below.

| | | IC50 ratio (IC50 |
|---|---|---|
| | | candidate/IC50 |
| FORMAT | | VTQ2101) |
| | VTQ2101 (LEAD) | 1 |
| 6 | ScFv-VTQ2101-LH-GS18-FC | 2.2 |
| 7 | ScFv-VTQ2101-LHλcap-GS18-FC | 1.7 |
| 3 | ScFv-VTQ2102-HLλcap-GS18-FC | 1.5 |
| 5 | scFab-VTQ2101-FC | 1.3 |

All these results show that different formats antigen-binding derivatives of VTQ2101 have a similar IC50 than VTQ2101.

### BIBLIOGRAPHIC REFERENCES

Bender B, Bodrogi L, Mayer B, Schneider Z, Zhao Y, Hammarstrom L, et al. Position independent and copy-number-related expression of the bovine neonatal Fc receptor alpha-chain in transgenic mice carrying a 102 kb BAC genomic fragment. Transgenic Res. (2007) 16:613-27. doi: 10.1007/s11248-007-9108-9
Cervenak J, Bender B, Schneider Z, Magna M, Carstea B V, Liliom K, et al. Neonatal FcR overexpression boosts humoral immune response in transgenic mice. J Immunol. (2011) 186:959-68. doi: 10.4049/jimmunol.1000353
Chothia C, Lesk a M. 1987 Canonical structures for the hypervariable regions of immunoglobulins. J Mol Biol. 196: 901-17.
Cibelli J B, Stice S L, Golueke P J, Kane J J, Jerry J, Blackwell C, Ponce de León F A, Robl J M. Cibelli J B, et al. 1998 Cloned transgenic calves produced from nonquiescent fetal fibroblasts. Science. 280:1256-8.
Edelman, G. M., Cunningham B A, Gall W E, Gottlieb P D, Rutischauser U, Waxdal M J. 1969. The covalent structure of an entire gammaG immunoglobulin molecule. Proc. Natl. Acad. USA, 63: 78-85eu Fan G, Wang Z, Hao M, Li J. 2015. Bispecific antibodies and their applications. J Hematol Oncol 8:130.
Fisher R, et al. Vaccine 21 (2003) 820-825.
Fowler D. M and Fields S. 2014, Deep mutational scanning: a new style of protein science. Nat. Methods, 11 (2014): 801-807
Gedon N K Y and Mueller R S. 2018, Atopic dermatitis in cats and dogs: a difficult disease for animals and owners. Clin Transl Allergy.; 8:41.
Giudicelli V., Chaume D. and Lefranc M.-P. 2005 IMGT/GENE-DB: a comprehensive database for human and mouse immunoglobulin and T cell receptor genes Nucl. Acids Res., 33(S1):D256-D261.
Gonzales A J, Fleck T J, Humphrey W R., et al. 2016. IL-31-induced pruritus in dogs: a novel experimental model to evaluate anti-pruritic effects of canine therapeutics. Veterinary Dermatology; 27 (1):34-e10
Gordon J. W, Scangos G. A, Plotkin D. J, Barbosa J. A, Ruddle F. H. 1980 Genetic transformation of mouse embryos by microinjection of purified DNA. Proc Natl Acad SCI USA; December 77(12):7380-4
Griffin C E and DeBoer D J. 2001, The ACVD task force on canine atopic dermatitis (XIV): clinical manifestations of canine atopic dermatitis. Vet Immunol Immunopathol.; 81(3-4):255-69.
Kabat E. 1991 and later editions, Sequences of proteins of immunological interest, 5^{th} Ed., U.S. Department of Health and Human Services, NIH
Larrick J W, Danielsson L, Brenner C A, Abrahamson M, Fry K E, Borrebaeck C A. Larrick J W, et al. 1989 Rapid cloning of rearranged immunoglobulin genes from human hybridoma cells using mixed primers and the polymerase chain reaction. Biochem Biophys Res Commun.; 160(3):1250-6.
Lefranc M P, Pommié C, Ruiz M, Giudicelli V, Foulquier E, Truong L, Thouvenin-Contet V, Lefranc G. Lefranc M P, et al. 2003 IMGT unique numbering for immunoglobulin and T cell receptor variable domains and Ig superfamily V-like domains. Dev Comp Immunol.; 27(1):55-77.
Lisa M. Bergeron, Erin E. McCandless, Steve Dunham, Bill Dunkle, Yaqi Zhu, John Shelly, Sandra Lightle, Andrea Gonzales, Graeme Bainbridge. 2014 Comparative functional characterization of canine IgG subclasses Vet. Immunol. Immunopathol 157 31-41.
Liu A Y, Robinson R R, Hellström K E, Murray E D Jr, Chang C P, Hellström I. Liu A Y, et al. 1987. Chimeric mouse-human IgG1 antibody that can mediate lysis of cancer cells. Proc Natl Acad Sci USA.; 84(10):3439-43.
Marsella R and De Benedetto A. 2017. Atopic Dermatitis in Animals and People: An Update and Comparative Review. Vet Sci.; 4(3):37.
Marsella R and Girolomoni, G. 2009 Canine models of atopic dermatitis: a useful tool with untapped potential. J Invest Dermatol.; 129(10):2351-7.
Marsella R. 2012 An update on the treatment of canine atopic dermatitis. Vet Med (Auckl).; 3:85-91.
Mineshige T, Kamiie J, Sugahara G, Shirota K. Mineshige T, et al. 2018. A study on periostin involvement in the pathophysiology of canine atopic skin. J Vet Med Sci.; 80(1):103-111.
Morrison S L, Johnson M J, Herzenberg L A, Oi V T et al. 1984. Chimeric human antibody molecules: mouse antigen-binding domains with human constant region domains. Proc Natl Acad Sci USA.; 81(21):6851-5.
Nakahara T, Furue M. 2018. Nemolizumab and Atopic Dermatitis: the Interaction Between Interleukin-31 and Interleukin-31 Receptor as a Potential Therapeutic Target for Pruritus in Patients With Atopic Dermatitis. Current Treatment Options in Allergy; Vol (5); Issue (4): 405-414
Needleman S B, Wunsch C D. Needleman S B, et al. 1970. A general method applicable to the search for similarities in the amino acid sequence of two proteins. J Mol Biol.; 48(3):443-53.
Nuttall T J, Marsella R, Rosenbaum M R, Gonzales A J, Fadok V A. et al. 2019. Update on pathogenesis, diagnosis, and treatment of atopic dermatitis in dogs. J Am Vet Med Assoc.; 254(11):1291-1300.
Olivry T, Mayhew D, Paps J S, Linder K E, Peredo C, Rajpal D, Hofland H, Cote-Sierra J et al. 2016. Early Activation of Th2/Th22 Inflammatory and Pruritogenic Pathways in Acute Canine Atopic Dermatitis Skin Lesions. J Invest Dermatol.; 136(10):1961-1969.
Olivry T, Saridomichelakis M, Nuttall T, Bensignor E, Griffin C. E, Hill P. B, International Committee on Allergic Diseases of Animals (ICADA). 2014. Validation of the canine atopic dermatitis extent and severity index (CADESI)-4, a simplified severity scale for assessing skin lesions of atopic dermatitis in dogs. Vet Dermatol. April; 25(2):77-85, e25
Riechmann L, Clark M, Waldmann H, Winter G. 1988. Reshaping human antibodies for therapy. Nature.; 332(6162):323-7.
Ryan T M, Ciavatta D J, Townes T M. 1997. Knockout-transgenic mouse model of sickle cell disease. Science; 278: 873-876
Santoro D. 2018. Evaluation of the secretion of antimicrobial peptides and antimicrobial effect of skin wash in atopic and healthy dogs: a preliminary study. Vet Dermatol.; 29(5):402-e132.
Santoro D. 2019. Therapies in Canine Atopic Dermatitis: An Update. Vet Clin North Am Small Anim Pract.; 49(1):9-26.
Schillberg S., Twyman R M, Fischer R. 2005.; Opportunities for recombinant antigen and antibody expression in transgenic plants—technology assessment. Vaccine.; 23(15):1764-9.
Stoger E, Sack M, Fischer R, Christou P. Stoger E, et al. 2002 Plantibodies: applications, advantages and bottlenecks. Curr Opin Biotechnol.; 13(2):161-6.
Tamamoto-Mochizuki C, Paps J S, Olivry T. 2019. Proactive maintenance therapy of canine atopic dermatitis with the anti-IL-31 lokivetmab. Can a monoclonal antibody blocking a single cytokine prevent allergy flares? Vet Dermatol. January 23. doi: 10.1111/vde.12715. Online ahead of print.
Tang L. 2001. Molecular cloning of canine IL-13 receptor alpha chain (alpha1 and alpha2) cDNAs and detection of corresponding mRNAs in canine tissues. Vet Immunol Immunopathol.; 79(3-4):181-95.
Whitelegg N, Rees A R. 2004 Antibody variable regions: toward a unified modeling method. Methods Mol Biol.; 248:51-91.
Whitelegg N R, Rees A R. 2000. WAM: an improved algorithm for modelling antibodies on the WEB. Protein Eng.; 13(12):819-24.
Wilhem S, Kovalik M, Favrot C. 2011 Breed-associated phenotypes in canine atopic dermatitis. Vet Dermatol.; 22(2):143-149.
Winter G. and Harris W J. (1993). Humanized antibodies. Trends Pharmacol., 14:139-43.

### PATENT LITERATURE CITATIONS

U.S. Pat. No. 4,816,567 (Genetech Inc, Recombinant immunoglobin preparations)
WO90/04036 (Medical Research Council—Agriculture and Food Research Council, Production of antibodies from transgenic animals)
WO95/17085 (Genzyme Transgenics Corp—Transgenic production of antibodies in milk)
WO00/26357 (Genzyme Transgenics Corp—Transgenic and clones mammals)
WO01/26455 (Genzyme Transgenics Corp—Method of producing a target molecule in a transgenic animal and purification of the target molecule)
WO2003/101485 (Macrogenics Inc—CD16A binding proteins and use for the treatment of immune disorders)
WO2004/050847 (GTC Biotherapeutics Inc—Modified antibodies stably produced in milk and methods of producing same)
WO2005/033281 (Sterrenbeld Biotechnologie North America Inc—A process of making transgenic mammals that produce exogenous proteins in milk and transgenic mammals produced thereby)
WO2007/048077 (GTC Biotherapeutics Inc—Antibodies with enhanced antibody-dependent cellular cytoxicity activity, methods of their production and use)
WO2007/106078 (GTC Biotherapeutics Inc—Clarification of transgenic milk using depth filtration)
WO2021/123094 (Intervet—Bispecific caninized antibodies and bispecific binding partners for treating atopic dermatitis)

1. An anti-canine interleukine-31-receptor A (cIL-31RA) monoclonal antibody or an antigen-binding fragment or an antigen-binding derivative thereof, wherein said antibody, antigen-binding fragment or antigen-binding derivative has the ability to inhibit the signaling pathway activated by the binding of canine IL-31 to canine IL-31RA in a cell-based assay consisting in mammalian cells expressing STAT3, a STAT3-inducible secreted embryonic alkaline phosphatase (SEAP), and canine IL-31RA and OSMRα with an IC50 at least 5-fold lower than a monoclonal anti-cIL-31RA antibody 28F12 comprising a variable region of the heavy chain (VH) consisting of SEQ ID NO: 1 and a variable region of the light chain (VL) consisting of SEQ ID NO: 2.

2. The anti-cIL-31RA monoclonal antibody, antigen-binding fragment or antigen-binding derivative according to claim 1, wherein the mammalian cells expressing STAT3, a STAT3-inducible secreted embryonic alkaline phosphatase (SEAP), and canine IL-31RA and OSMRα are HEK293 cells transfected by expression vectors of STAT3, a STAT3-inducible secreted embryonic alkaline phosphatase (SEAP), and canine IL-31RA and OSMRα.

3. The anti-cIL-31RA monoclonal antibody, antigen-binding fragment or antigen-binding derivative according to claim 1, wherein said antibody, antigen-binding fragment or antigen-binding derivative competes for binding to cIL-31RA with a caninized monoclonal anti-cIL-31RA monoclonal antibody comprising:
a) a variable region of the heavy chain (VH) consisting of SEQ ID NO: 3, and
b) a variable region of the light chain (VL) consisting of SEQ ID NO: 4.

4. The anti-cIL-31RA monoclonal antibody, antigen-binding fragment or antigen-binding derivative according to claim 1, wherein said antibody, antigen-binding fragment or antigen-binding derivative comprises heavy and light chains respectively comprising CDR-H and CDR-L with the following amino acid sequences according to Kabat numbering:
CDR-H-1: X1SFIH (SEQ ID NO: 5), wherein X1 is selected from S or D,
CDR-H-2: X2IDPX3X4GX5TEYX6X7X8FQG (SEQ ID NO: 6), wherein:
X2 is selected from R and Y,
X3 is selected from A and L,
X4 is selected from F, N and Q,
X5 is selected from A, N and G,
X6 is selected from D and N,
X7 is selected from A and P,
X8 is selected from A, N and V,
CDR-H-3: YX9YX10X11SHFDX12 (SEQ ID NO: 7), wherein:
X9 is selected from H and Y,
X10 is selected from A and G,
X11 is selected from A, R, N, T and Q,
X12 is selected from A and C
CDR-L-1: KX13SQSVTNDX14T (SEQ ID NO: 8), wherein:
X13 is selected from S and A,
X14 is selected from L and V,
CDR-L-2: YASX15RYX16 (SEQ ID NO: 9), wherein:
X15 is selected from Q, N and I,
X16 is selected from T, S and P, and
CDR-L-3: QQDYX17SPFT (SEQ ID NO: 10), wherein X17 is selected from A and S.

5. The anti-cIL-31RA monoclonal antibody, antigen-binding fragment or antigen-binding derivative according to claim 1, which is a chimeric or a caninized antibody.

6. The anti-cIL-31RA monoclonal antibody, antigen-binding fragment or antigen-binding derivative according to claim 5, which is a chimeric antibody comprising heavy and light chains respectively comprising CDR-H and CDR-L with the following amino acid sequences according to Kabat numbering:
CDR-H-1:
(SEQ ID NO: 11)
DSFIH,
CDR-H-2:
(SEQ ID NO: 12)
RIDPANGNTEYDPNFQG,
CDR-H-3:
(SEQ ID NO: 13)
YYYGNSHFDC
or
(SEQ ID NO: 93)
YYYGNSHFDA,
CDR-L-1:
(SEQ ID NO: 14)
KASQSVINDVT,
CDR-L-2:
(SEQ ID NO: 15)
YASNRYT,
and
CDR-L-3:
(SEQ ID NO: 16)
QQDYSSPFT.

7. The anti-cIL-31RA monoclonal antibody, antigen-binding fragment of antigen-binding derivative according to claim 6, which comprises:
a) a variable region of the heavy chain (VH) with at least 80% identity with SEQ ID NO: 17, or a variable region of the heavy chain (VH) with at least 80% identity with SEQ ID NO: 94, and/or
b) a variable region of the light chain (VL) with at least 80% identity with SEQ ID NO: 18.

8. The anti-cIL-31RA monoclonal antibody, antigen-binding fragment or antigen-binding derivative according to claim 5, which is a caninized antibody comprising heavy and light chains respectively comprising CDR-H and CDR-L with the following amino acid sequences according to Kabat numbering:
CDR-H-1: X1SFIH (SEQ ID NO: 5), wherein X1 is selected from S or D,
CDR-H-2: X2IDPX3X4GX5TEYX6X7X8FQG (SEQ ID NO: 20), wherein:
X2 is selected from R and Y,
X3 is selected from A and L,
X4 is selected from F and Q,
X5 is selected from A, N and G,
X6 is selected from D and N,
X7 is selected from A and P,
X8 is selected from A, N and V,
CDR-H-3: YX9YX10X11SHFDA (SEQ ID NO: 21), wherein:
X9 is selected from H and Y,
X10 is selected from A and G,
X11 is selected from A, R, T and Q,
CDR-L-1: KX13SQSVTNDX14T (SEQ ID NO: 8), wherein:
X13 is selected from S and A,
X14 is selected from L and V,
CDR-L-2: YASX15RYX16 (SEQ ID NO: 9), wherein:
X15 is selected from Q, N and I,
X16 is selected from T, S and P, and
CDR-L-3: QQDYX17SPFT (SEQ ID NO: 10), wherein X17 is selected from A and S.

9. The anti-cIL-31RA monoclonal antibody, antigen-binding fragment or antigen-binding derivative according to claim 8, which is a caninized antibody comprising heavy and light chains respectively comprising CDR-H and CDR-L with one of the following amino acid sequences sets a) to n) according to Kabat numbering:
a) 8D3-VHL/8D3-VLH:
CDR-H-1:
(SEQ ID NO: 22)
SSFIH,
CDR-H-2:
(SEQ ID NO: 23)
RIDPAFGATEYNPAFQG,
CDR-H-3:
(SEQ ID NO: 24)
YHYAASHFDA,
CDR-L-1:
(SEQ ID NO: 25)
KSSQSVINDLT,
CDR-L-2:
(SEQ ID NO: 26)
YASQRYT,
and
CDR-L-3:
(SEQ ID NO: 27)
QQDYASPFT;
b) 8D3-clone7v2-VH/8D3-clone 7-VL:
CDR-H-1:
(SEQ ID NO: 28)
SSFIH,
CDR-H-2:
(SEQ ID NO: 29)
RIDPLQGGTEYNPVFQG,
CDR-H-3:
(SEQ ID NO: 30)
YYYAQSHFDA,
CDR-L-1:
(SEQ ID NO: 31)
KSSQSVTNDLT,
CDR-L-2:
(SEQ ID NO: 32)
YASQRYT,
and
CDR-L-3:
(SEQ ID NO: 33)
QQDYSSPFT;
c) 8D3-VH-L/8D3-VL-Ev2:
CDR-H-1:
(SEQ ID NO: 22)
SSFIH,
CDR-H-2:
(SEQ ID NO: 23)
RIDPAFGATEYNPAFQG,
CDR-H-3:
(SEQ ID NO: 24)
YHYAASHFDA,
CDR-L-1:
(SEQ ID NO: 34)
KSSQSVTNDVT,
CDR-L-2:
(SEQ ID NO: 35)
YASQRYS,
and
CDR-L-3:
(SEQ ID NO: 36)
QQDYASPFT;
d) 8D3-VH-L/8D3-VL-G:
CDR-H-1:
(SEQ ID NO: 22)
SSFIH,
CDR-H-2:
(SEQ ID NO: 23)
RIDPAFGATEYNPAFQG,
CDR-H-3:
(SEQ ID NO: 24)
YHYAASHFDA,
CDR-L-1:
(SEQ ID NO: 37)
KSSQSVINDLT,
CDR-L-2:
(SEQ ID NO: 38)
YASQRYP,
and
CDR-L-3:
(SEQ ID NO: 39)
QQDYASPFT;
e) 8D3-VH-Lv2/8D3-VL-Ev2:
CDR-H-1:
(SEQ ID NO: 40)
SSFIH,
CDR-H-2:
(SEQ ID NO: 41)
RIDPLQGATEYNPVFQG,
CDR-H-3:
(SEQ ID NO: 42)
YHYAQSHFDA,
CDR-L-1:
(SEQ ID NO: 34)
KSSQSVINDVT,
CDR-L-2:
(SEQ ID NO: 35)
YASQRYS,
and
CDR-L-3:
(SEQ ID NO: 36)
QQDYASPFT;
f) 8D3-VH-Lv2/8D3-VL-G:
CDR-H-1:
(SEQ ID NO: 40)
SSFIH,
CDR-H-2:
(SEQ ID NO: 41)
RIDPLQGATEYNPVFQG,
CDR-H-3:
(SEQ ID NO: 42)
YHYAQSHFDA,
CDR-L-1:
(SEQ ID NO: 37)
KSSQSVINDLT,
CDR-L-2:
(SEQ ID NO: 38)
YASQRYP,
and
CDR-L-3:
(SEQ ID NO: 39)
QQDYASPFT;
g) 8D3-VH-N/8D3-VL-E:
CDR-H-1:
(SEQ ID NO: 43)
SSFIH,
CDR-H-2:
(SEQ ID NO: 44)
YIDPLQGGTEYNPVFQG,
CDR-H-3:
(SEQ ID NO: 45)
YYYAQSHFDA,
CDR-L-1:
(SEQ ID NO: 46)
KSSQSVINDVT,
CDR-L-2:
(SEQ ID NO: 47)
YASIRYS,
and
CDR-L-3:
(SEQ ID NO: 48)
QQDYASPFT;
h) 8D3-VH-H/8D3-VL-Ev2:
CDR-H-1:
(SEQ ID NO: 49)
DSFIH,
CDR-H-2:
(SEQ ID NO: 50)
RIDPAQGATEYDANFQG,
CDR-H-3:
(SEQ ID NO: 51)
YYYGASHFDA,
CDR-L-1:
(SEQ ID NO: 34)
KSSQSVINDVT,
CDR-L-2:
(SEQ ID NO: 35)
YASQRYS,
and
CDR-L-3:
(SEQ ID NO: 36)
QQDYASPFT;
i) 8D3-VH-Lv2/8D3-VL-E:
CDR-H-1:
(SEQ ID NO: 40)
SSFIH,
CDR-H-2:
(SEQ ID NO: 41)
RIDPLQGATEYNPVFQG,
CDR-H-3:
(SEQ ID NO: 42)
YHYAQSHFDA,
CDR-L-1:
(SEQ ID NO: 46)
KSSQSVINDVT,
CDR-L-2:
(SEQ ID NO: 47)
YASIRYS,
and
CDR-L-3:
(SEQ ID NO: 48)
QQDYASPFT;
j) 8D3-VH-Lv2/8D3-VL-H:
CDR-H-1:
(SEQ ID NO: 40)
SSFIH,
CDR-H-2:
(SEQ ID NO: 41)
RIDPLQGATEYNPVFQG,
CDR-H-3:
(SEQ ID NO: 42)
YHYAQSHFDA,
CDR-L-1:
(SEQ ID NO: 25)
KSSQSVTNDLT,
CDR-L-2:
(SEQ ID NO: 26)
YASQRYT,
and
CDR-L-3:
(SEQ ID NO: 27)
QQDYASPFT;
k) 8D3-VH-N/8D3-VL-Ev2:
CDR-H-1:
(SEQ ID NO: 43)
SSFIH,
CDR-H-2:
(SEQ ID NO: 44)
YIDPLQGGTEYNPVFQG,
CDR-H-3:
(SEQ ID NO: 45)
YYYAQSHFDA,
CDR-L-1:
(SEQ ID NO: 34)
KSSQSVINDVT,
CDR-L-2:
(SEQ ID NO: 35)
YASQRYS,
and
CDR-L-3:
(SEQ ID NO: 36)
QQDYASPFT;
l) 8D3-VH-518/8D3-VL-A:
CDR-H-1:
(SEQ ID NO: 52)
DSFIH,
CDR-H-2:
(SEQ ID NO: 53)
YIDPLQGNTEYDPVFQG,
CDR-H-3:
(SEQ ID NO: 54)
YYYARSHFDA,
CDR-L-1:
(SEQ ID NO: 55)
KASQSVINDVT,
CDR-L-2:
(SEQ ID NO: 56)
YASNRYT,
and
CDR-L-3:
(SEQ ID NO: 57)
QQDYSSPFT;
m) 8D3-VH-518/8D3-VL-Ev2:
CDR-H-1:
(SEQ ID NO: 52)
DSFIH,
CDR-H-2:
(SEQ ID NO: 53)
YIDPLQGNTEYDPVFQG,
CDR-H-3:
(SEQ ID NO: 54)
YYYARSHFDA,
CDR-L-1:
(SEQ ID NO: 34)
KSSQSVINDVT,
CDR-L-2:
(SEQ ID NO: 35)
YASQRYS,
and
CDR-L-3:
(SEQ ID NO: 36)
QQDYASPFT;
and
8D3-VH-518H/8D3-VL-A:
CDR-H-1:
(SEQ ID NO: 58)
SSFIH,
CDR-H-2:
(SEQ ID NO: 59)
YIDPLQGGTEYNPVFQG,
CDR-H-3:
(SEQ ID NO: 60)
YHYATSHFDA,
CDR-L-1:
(SEQ ID NO: 55)
KASQSVINDVT,
CDR-L-2:
(SEQ ID NO: 56)
YASNRYT,
and
CDR-L-3:
(SEQ ID NO: 57)
QQDYSSPFT.

10. The anti-cIL-31RA monoclonal antibody, antigen-binding fragment or antigen-binding derivative according to claim 9, which comprises one of the following amino acid sequences sets a) to n):
a1) 8D3-VHL/8D3-VLH:
a variable region of the heavy chain (VH) with at least 80% identity with SEQ ID NO: 3, and/or
a variable region of the light chain (VL) with at least 80% identity with SEQ ID NO: 4;
a2) 8D3-VHL/8D3-VLHλcap:
a variable region of the heavy chain (VH) with at least 80% identity with SEQ ID NO: 3, and/or
a variable region of the light chain (VL) with at least 80% identity with SEQ ID NO: 113;
b) 8D3-clone7v2-VH/8D3-clone 7-VL:
a variable region of the heavy chain (VH) with at least 80% identity with SEQ ID NO: 61, and/or
a variable region of the light chain (VL) with at least 80% identity with SEQ ID NO: 67;
c) 8D3-VH-L/8D3-VL-Ev2:
a variable region of the heavy chain (VH) with at least 80% identity with SEQ ID NO: 3, and/or
a variable region of the light chain (VL) with at least 80% identity with SEQ ID NO: 68;
d) 8D3-VH-L/8D3-VL-G:
a variable region of the heavy chain (VH) with at least 80% identity with SEQ ID NO: 3, and/or
a variable region of the light chain (VL) with at least 80% identity with SEQ ID NO: 69;
e) 8D3-VH-Lv2/8D3-VL-Ev2:
a variable region of the heavy chain (VH) with at least 80% identity with SEQ ID NO: 62, and/or
a variable region of the light chain (VL) with at least 80% identity with SEQ ID NO: 68;
f) 8D3-VH-Lv2/8D3-VL-G:
a variable region of the heavy chain (VH) with at least 80% identity with SEQ ID NO: 62, and/or
a variable region of the light chain (VL) with at least 80% identity with SEQ ID NO: 69;
g) 8D3-VH-N/8D3-VL-E:
a variable region of the heavy chain (VH) with at least 80% identity with SEQ ID NO: 63, and/or
a variable region of the light chain (VL) with at least 80% identity with SEQ ID NO: 70;
h) 8D3-VH-H/8D3-VL-Ev2:
a variable region of the heavy chain (VH) with at least 80% identity with SEQ ID NO: 64, and/or
a variable region of the light chain (VL) with at least 80% identity with SEQ ID NO: 68;
i) 8D3-VH-Lv2/8D3-VL-E:
a variable region of the heavy chain (VH) with at least 80% identity with SEQ ID NO: 62, and/or
a variable region of the light chain (VL) with at least 80% identity with SEQ ID NO: 70;
j) 8D3-VH-Lv2/8D3-VL-H:
a variable region of the heavy chain (VH) with at least 80% identity with SEQ ID NO: 62, and/or
a variable region of the light chain (VL) with at least 80% identity with SEQ ID NO: 4;
k) 8D3-VH-N/8D3-VL-Ev2:
a variable region of the heavy chain (VH) with at least 80% identity with SEQ ID NO: 63, and/or
a variable region of the light chain (VL) with at least 80% identity with SEQ ID NO: 68;
l) 8D3-VH-518/8D3-VL-A:
a variable region of the heavy chain (VH) with at least 80% identity with SEQ ID NO: 65, and/or
a variable region of the light chain (VL) with at least 80% identity with SEQ ID NO: 71;
m) 8D3-VH-518/8D3-VL-Ev2:
a variable region of the heavy chain (VH) with at least 80% identity with SEQ ID NO: 65, and/or
a variable region of the light chain (VL) with at least 80% identity with SEQ ID NO: 68; and
n) 8D3-VH-518H/8D3-VL-A:
a variable region of the heavy chain (VH) with at least 80% identity with SEQ ID NO: 66, and/or
a variable region of the light chain (VL) with at least 80% identity with SEQ ID NO: 71.

11. An antigen-binding derivative according to claim 1, which comprises any one of the amino acid sequences SEQ ID NO: 106 to SEQ ID NO: 112, fused in C-terminal with an Fc fragment.

12. A bispecific antibody comprising a first antigen-binding fragment or antigen-binding derivative according to claim 1 and a second antigen-binding fragment or antigen-binding derivative directed to one other target relevant for treating atopic dermatitis.

13. A nucleic acid or a combination of two nucleic acids encoding the antibody, antigen-binding fragment or antigen-binding derivative according to claim 1 or a bispecific antibody comprising a first antigen-binding fragment or antigen-binding derivative according to claim 1 and a second antigen-binding fragment or antigen-binding derivative directed to one other target relevant for treating atopic dermatitis.

14. A vector or a combination of two vectors comprising the nucleic acid(s) according to claim 13.

15. A host cell comprising the nucleic acid(s) according to claim 13 or a vector or a combination of two vectors comprising it.

16. (canceled)

17. A method for the treatment and/or prevention of itch and/or inflammatory skin due to atopic dermatitis and/or allergies in dogs, comprising administering to dogs an effective amount of the anti-cIL-31RA monoclonal antibody, antigen-binding fragment or antigen-binding derivative according to claim 1 or a bispecific antibody comprising a first antigen-binding fragment or antigen-binding derivative according to claim 1 and a second antigen-binding fragment or antigen-binding derivative directed to one other target relevant for treating atopic dermatitis.

18. The anti-cIL-31RA monoclonal antibody, antigen-binding fragment or antigen-binding derivative according to claim 2, wherein the mammalian cells expressing STAT3, a STAT3-inducible secreted embryonic alkaline phosphatase (SEAP), and canine IL-31RA and OSMRα are cells deposited at CNCM on Dec. 8, 2021 under number I-5792.

19. The anti-cIL-31RA monoclonal antibody, antigen-binding fragment or antigen-binding derivative according to claim 8, wherein
the heavy chain variable region of said antibody, antigen-binding fragment or antigen-binding derivative further comprises:
an amino acid selected from F, I, and L at position H67 according to Kabat numbering,
an amino acid selected from A and T at position H74 according to Kabat numbering,
an amino acid selected from A and V at position H78 according to Kabat numbering, and
an amino acid selected from S and T at position H87 according to Kabat numbering; and
the light chain variable region of said antibody further comprises:
an amino acid selected from G and V at position L13 according to Kabat numbering,
an amino acid selected from A and V at position L15 according to Kabat numbering,
an amino acid selected from W and Q at position L38 according to Kabat numbering,
an amino acid selected from R and A at position L43 according to Kabat numbering,
an amino acid selected from T and H at position L49 according to Kabat numbering,
an amino acid selected from S and Y at position L67 according to Kabat numbering,
an amino acid selected from F and L at position L73 according to Kabat numbering, and
an amino acid selected from D and V at position L85 according to Kabat numbering.

20. The antigen-binding derivative according to claim 11, which is selected from the amino acid sequences SEQ ID NO:97 to SEQ ID NO: 103.

21. The method of claim 17 for the treatment of canine atopic dermatitis.


SEQUENCE LISTING (121 sequences)

SEQ ID NO: 1 - protein, 122 aa, synthetic construct
  REGION 1..122: note = 28F12 variable region of the heavy chain (VH)
EVQLVESDGGLAQPGRSLKLSCAASGFTFSDYYMAWVRQAPTKGLEWVATISYDGSSTYYRDSVRGRFTISRDNAKSTLYLQMDSLRSEDTATYYCARGPLTDWAPNWFAYWGQGTLVTVSS

SEQ ID NO: 2 - protein, 107 aa, synthetic construct
  REGION 1..107: note = 28F12 variable region of the light chain (VL)
DIQMTQSPASLSASLGETVTIQCQTSEDIYSGLAWYQQKPGKSPQFLIYGASRLEDGVPSRFSGSGSGTQYSLKISSMQTEDEGVYFCQQGLKYPNTFGAGTKLELK

SEQ ID NO: 3 - protein, 119 aa, synthetic construct
  REGION 1..119: note = Can8D3-VHL variable region of the heavy chain (VH)
EVTLQESGPGLVKPSQTLSLTCVASGFSIKSSFIHWLRQRPGRGLEWIGRIDPAFGATEYNPAFQGRFSITADTAKNQASLQLSSMTTEDSAVYYCARYHYAASHFDAWGQGTLVTVSS

SEQ ID NO: 4 - protein, 107 aa, synthetic construct
  REGION 1..107: note = Can8D3-VLH variable region of the light chain (VL)
RIVMTQSPGSLAGSVGESVSINCKSSQSVTNDLTWYQQKPGEAPKVLITYASQRYTGVPARFSGSGYGTDFTLTINNLQAEDVGDYFCQQDYASPFTFGQGTKLEIK

SEQ ID NO: 5 - protein, 5 aa, synthetic construct
  REGION 1..5: note = Chim+Can CDR-H-1
  VARIANT 1: note = X is selected from S or D
XSFIH

SEQ ID NO: 6 - protein, 17 aa, synthetic construct
  REGION 1..17: note = Chim+Can CDR-H-2
  VARIANT 1: note = X is selected from R and Y
  VARIANT 5: note = X is selected from A and L
  VARIANT 6: note = X is selected from F, N and Q
  VARIANT 8: note = X is selected from A, N and G
  VARIANT 12: note = X is selected from D and N
  VARIANT 13: note = X is selected from A and P
  VARIANT 14: note = X is selected from A, N and V
XIDPXXGXTEYXXXFQG

SEQ ID NO: 7 - protein, 10 aa, synthetic construct
  REGION 1..10: note = Chim+Can CDR-H-3
  VARIANT 2: note = X is selected from H and Y
  VARIANT 4: note = X is selected from A and G
  VARIANT 5: note = X is selected from A, R, N, T and Q
  VARIANT 10: note = X is selected from A and C
YXYXXSHFDX

SEQ ID NO: 8 - protein, 11 aa, synthetic construct
  REGION 1..11: note = Chim+CanCDR-L-1 / Can CDR-L-1
  VARIANT 2: note = X is selected from S and A
  VARIANT 10: note = X is selected from L and V
KXSQSVTNDXT

SEQ ID NO: 9 - protein, 7 aa, synthetic construct
  REGION 1..7: note = Chim+CanCDR-L-2 / Can CDR-L-2
  VARIANT 4: note = X is selected from Q, N and I
  VARIANT 7: note = X is selected from T, S and P
YASXRYX

SEQ ID NO: 10 - protein, 9 aa, synthetic construct
  REGION 1..9: note = Chim+Can CDR-L-3 / Can CDR-L-3
  VARIANT 5: note = X is selected from A and S
QQDYXSPFT

SEQ ID NO: 11 - protein, 5 aa, synthetic construct
  REGION 1..5: note = Chim CDR-H-1
DSFIH

SEQ ID NO: 12 - protein, 17 aa, synthetic construct
  REGION 1..17: note = Chim CDR-H-2
RIDPANGNTEYDPNFQG

SEQ ID NO: 13 - protein, 10 aa, synthetic construct
  REGION 1..10: note = Chim CDR-H-3
YYYGNSHFDC

SEQ ID NO: 14 - protein, 11 aa, synthetic construct
  REGION 1..11: note = Chim CDR-L-1
KASQSVTNDVT

SEQ ID NO: 15 - protein, 7 aa, synthetic construct
  REGION 1..7: note = Chim CDR-L-2
YASNRYT

SEQ ID NO: 16 - protein, 9 aa, synthetic construct
  REGION 1..9: note = Chim CDR-L-3
QQDYSSPFT

SEQ ID NO: 17 - protein, 119 aa, synthetic construct
  REGION 1..119: note = Chim VH
EVQLQQSGAELVKPGASVKLSCTASGFNIKDSFIHWLKQRPEQGLEWIGRIDPANGNTEYDPNFQGKDTITADTSSNTAYLQVSSLTSEDTAVYYCARYYYGNSHFDCWGQGTTLTVSS

SEQ ID NO: 18 - protein, 107 aa, synthetic construct
  REGION 1..107: note = Chim VL
RIVMNQTPKFLPISAGDRVIITCKASQSVTNDVTWYQQKPGQSPKVLIHYASNRYTGVPDRFTGSGYGTDFTFTISTVQAEDLAVYFCQQDYSSPFTFGSGTKLEIK

SEQ ID NO: 19 - protein, 5 aa, synthetic construct
  REGION 1..5: note = Can CDR-H-1
SSFIH

SEQ ID NO: 20 - protein, 17 aa, synthetic construct
  REGION 1..17: note = Can CDR-H-2 (consensus)
  VARIANT 1: note = X is selected from R and Y
  VARIANT 5: note = X is selected from A and L
  VARIANT 6: note = X is selected from F and Q
  VARIANT 8: note = X is selected from A, N and G
  VARIANT 12: note = X is selected from D and N
  VARIANT 13: note = X is selected from A and P
  VARIANT 14: note = X is selected from A, N and V
XIDPXXGXTEYXXXFQG

SEQ ID NO: 21 - protein, 10 aa, synthetic construct
  REGION 1..10: note = Can CDR-H-3 (consensus)
  VARIANT 2: note = X is selected from H and Y
  VARIANT 4: note = X is selected from A and G
  VARIANT 5: note = X is selected from A, R, T and Q
YXYXXSHFDA

SEQ ID NO: 22 - protein, 5 aa, synthetic construct
  REGION 1..5: note = 8D3-VHL- CDR-H-1
SSFIH

SEQ ID NO: 23 - protein, 17 aa, synthetic construct
  REGION 1..17: note = 8D3-VHL- CDR-H-2
RIDPAFGATEYNPAFQG

SEQ ID NO: 24 - protein, 10 aa, synthetic construct
  REGION 1..10: note = 8D3-VHL- CDR-H-3
YHYAASHFDA

SEQ ID NO: 25 - protein, 11 aa, synthetic construct
  REGION 1..11: note = 8D3-VHL- CDR-L-1
KSSQSVTNDLT

SEQ ID NO: 26 - protein, 7 aa, synthetic construct
  REGION 1..7: note = 8D3-VHL- CDR-L-2
YASQRYT

SEQ ID NO: 27 - protein, 9 aa, synthetic construct
  REGION 1..9: note = 8D3-VHL- CDR-L-3
QQDYASPFT

SEQ ID NO: 28 - protein, 5 aa, synthetic construct
  REGION 1..5: note = 8D3-clone7v2-VH-CDR-H-1
SSFIH

SEQ ID NO: 29 - protein, 17 aa, synthetic construct
  REGION 1..17: note = 8D3-clone7v2-VH-CDR-H-2
RIDPLQGGTEYNPVFQG

SEQ ID NO: 30 - protein, 10 aa, synthetic construct
  REGION 1..10: note = 8D3-clone7v2-VH-CDR-H-3
YYYAQSHFDA

SEQ ID NO: 31 - protein, 11 aa, synthetic construct
  REGION 1..11: note = 8D3-clone 7-VL-CDR-L-1
KSSQSVTNDLT

SEQ ID NO: 32 - protein, 7 aa, synthetic construct
  REGION 1..7: note = 8D3-clone 7-VL-CDR-L-2
YASQRYT

SEQ ID NO: 33 - protein, 9 aa, synthetic construct
  REGION 1..9: note = 8D3-clone 7-VL-CDR-L-3
QQDYSSPFT

SEQ ID NO: 34 - protein, 11 aa, synthetic construct
  REGION 1..11: note = 8D3-VL-Ev2-CDR-L-1
KSSQSVTNDVT

SEQ ID NO: 35 - protein, 7 aa, synthetic construct
  REGION 1..7: note = 8D3-VL-Ev2-CDR-L-2
YASQRYS

SEQ ID NO: 36 - protein, 9 aa, synthetic construct
  REGION 1..9: note = 8D3-VL-Ev2-CDR-L-3
QQDYASPFT

SEQ ID NO: 37 - protein, 11 aa, synthetic construct
  REGION 1..11: note = 8D3-VL-G-CDR-L-1
KSSQSVTNDLT

SEQ ID NO: 38 - protein, 7 aa, synthetic construct
  REGION 1..7: note = 8D3-VL-G-CDR-L-2
YASQRYP

SEQ ID NO: 39 - protein, 9 aa, synthetic construct
  REGION 1..9: note = 8D3-VL-G-CDR-L-3
QQDYASPFT

SEQ ID NO: 40 - protein, 5 aa, synthetic construct
  REGION 1..5: note = 8D3-VH-Lv2-CDR-H-1
SSFIH

SEQ ID NO: 41 - protein, 17 aa, synthetic construct
  REGION 1..17: note = 8D3-VH-Lv2-CDR-H-2
RIDPLQGATEYNPVFQG

SEQ ID NO: 42 - protein, 10 aa, synthetic construct
  REGION 1..10: note = 8D3-VH-Lv2-CDR-H-3
YHYAQSHFDA

SEQ ID NO: 43 - protein, 5 aa, synthetic construct
  REGION 1..5: note = 8D3-VH-N-CDR-H-1
SSFIH

SEQ ID NO: 44 - protein, 17 aa, synthetic construct
  REGION 1..17: note = 8D3-VH-N-CDR-H-2
YIDPLQGGTEYNPVFQG

SEQ ID NO: 45 - protein, 10 aa, synthetic construct
  REGION 1..10: note = 8D3-VH-N-CDR-H-3
YYYAQSHFDA

SEQ ID NO: 46 - protein, 11 aa, synthetic construct
  REGION 1..11: note = 8D3-VL-E-CDR-L-1
KSSQSVTNDVT

SEQ ID NO: 47 - protein, 7 aa, synthetic construct
  REGION 1..7: note = 8D3-VL-E-CDR-L-2
YASIRYS

SEQ ID NO: 48 - protein, 9 aa, synthetic construct
  REGION 1..9: note = 8D3-VL-E-CDR-L-3
QQDYASPFT

SEQ ID NO: 49 - protein, 5 aa, synthetic construct
  REGION 1..5: note = 8D3-VH-H-CDR-H-1
DSFIH

SEQ ID NO: 50 - protein, 17 aa, synthetic construct
  REGION 1..17: note = 8D3-VH-H-CDR-H-2
RIDPAQGATEYDANFQG

SEQ ID NO: 51 - protein, 10 aa, synthetic construct
  REGION 1..10: note = 8D3-VH-H-CDR-H-3
YYYGASHFDA

SEQ ID NO: 52 - protein, 5 aa, synthetic construct
  REGION 1..5: note = 8D3-VH-518-CDR-H-1
DSFIH

SEQ ID NO: 53 - protein, 17 aa, synthetic construct
  REGION 1..17: note = 8D3-VH-518-CDR-H-2
YIDPLQGNTEYDPVFQG

SEQ ID NO: 54 - protein, 10 aa, synthetic construct
  REGION 1..10: note = 8D3-VH-518-CDR-H-3
YYYARSHFDA

SEQ ID NO: 55 - protein, 11 aa, synthetic construct
  REGION 1..11: note = 8D3-VL-A-CDR-L-1
KASQSVTNDVT

SEQ ID NO: 56 - protein, 7 aa, synthetic construct
  REGION 1..7: note = 8D3-VL-A-CDR-L-2
YASNRYT

SEQ ID NO: 57 - protein, 9 aa, synthetic construct
  REGION 1..9: note = 8D3-VL-A-CDR-L-3
QQDYSSPFT

SEQ ID NO: 58 - protein, 5 aa, synthetic construct
  REGION 1..5: note = 8D3-VH-518H-CDR-H-1
SSFIH

SEQ ID NO: 59 - protein, 17 aa, synthetic construct
  REGION 1..17: note = 8D3-VH-518H-CDR-H-2
YIDPLQGGTEYNPVFQG

SEQ ID NO: 60 - protein, 10 aa, synthetic construct
  REGION 1..10: note = 8D3-VH-518H-CDR-H-3
YHYATSHFDA

SEQ ID NO: 61 - protein, 119 aa, synthetic construct
  REGION 1..119: note = 8D3-clone7v2-VH
EVTLQESGPGLVKPSQTLSLTCVASGFSIKSSFIHWLRQRPGRGLEWIGRIDPLQGGTEYNPVFQGRISITADTTKNQVSLQLSSMTTEDTAVYYCARYYYAQSHFDAWGQGTLVTVSS

SEQ ID NO: 62 - protein, 119 aa, synthetic construct
  REGION 1..119: note = 8D3-VH-Lv2
EVTLQESGPGLVKPSQTLSLTCVASGFSIKSSFIHWLRQRPGRGLEWIGRIDPLQGATEYNPVFQGRLSITADTAKNQVSLQLSSMTTEDTAVYYCARYHYAQSHFDAWGQGTLVTVSS

SEQ ID NO: 63 - protein, 119 aa, synthetic construct
  REGION 1..119: note = 8D3-VH-N
EVTLQESGPGLVKPSQTLSLTCVASGFSIKSSFIHWLRQRPGRGLEWIGYIDPLQGGTEYNPVFQGRISITADTAKNQVSLQLSSMTTEDTAVYYCARYYYAQSHFDAWGQGTLVTVSS

SEQ ID NO: 64 - protein, 119 aa, synthetic construct
  REGION 1..119: note = 8D3-VH-H
EVTLQESGPGLVKPSQTLSLTCVASGFSIKDSFIHWLRQRPGRGLEWIGRIDPAQGATEYDANFQGRISITADTAKNQASLQLSSMTTEDTAVYYCARYYYGASHFDAWGQGTLVTVSS

SEQ ID NO: 65 - protein, 118 aa, synthetic construct
  REGION 1..118: note = 8D3-VH-518
VTLQESGPGLVKPSQTLSLTCVASGFSIKDSFIHWLRQRPGRGLEWIGYIDPLQGNTEYDPVFQGRLSITADTAKNQVSLQLSSMTTEDTAVYYCARYYYARSHFDAWGQGTLVTVSS

SEQ ID NO: 66 - protein, 119 aa, synthetic construct
  REGION 1..119: note = 8D3-VH-518H
EVTLQESGPGLVKPSQTLSLTCVASGFSIKSSFIHWLRQRPGRGLEWIGYIDPLQGGTEYNPVFQGRLSITADTAKNQVSLQLSSMTTEDTAVYYCARYHYATSHFDAWGQGTLVTVSS

SEQ ID NO: 67 - protein, 107 aa, synthetic construct
  REGION 1..107: note = 8D3-clone 7-VL
RIVMTQSPGSLAGSAGESVSINCKSSQSVTNDLTWYQQKPGEAPKVLIHYASQRYTGVPARFSGSGYGTDFTLTINNLQAEDVGVYFCQQDYSSPFTFGQGTKLEIK

SEQ ID NO: 68 - protein, 107 aa, synthetic construct
  REGION 1..107: note = 8D3-VL-Ev2
RIVMTQSPGSLAGSVGESVSINCKSSQSVTNDVTWYQQKPGEAPKVLIHYASQRYSGVPARFSGSGYGTDFTLTINNLQAEDVGDYFCQQDYASPFTFGQGTKLEIK

SEQ ID NO: 69 - protein, 107 aa, synthetic construct
  REGION 1..107: note = 8D3-VL-G
RIVMTQSPGSLAGSVGESVSINCKSSQSVTNDLTWYQQKPGEAPKVLIHYASQRYPGVPARFSGSGYGTDFTLTINNLQAEDVGDYFCQQDYASPFTFGQGTKLEIK

SEQ ID NO: 70 - protein, 107 aa, synthetic construct
  REGION 1..107: note = 8D3-VL-E
RIVMTQSPGSLAVSVGESVSINCKSSQSVTNDVTWYQWKPGEAPKVLITYASIRYSGVPARFSGSGYGTDFTLTINNLQAEDVGDYFCQQDYASPFTFGQGTKLEIK

SEQ ID NO: 71 - protein, 107 aa, synthetic construct
  REGION 1..107: note = 8D3-VL-A
RIVMTQSPGSLAGSAGESVSINCKASQSVTNDVTWYQQKPGERPKVLIHYASNRYTGVPARFSGSGSGTDFTFTINNLQAEDVGDYFCQQDYSSPFTFGQGTKLEIK

SEQ ID NO: 72 - protein, 334 aa, synthetic construct
  REGION 1..334: note = wild type canine IgGB constant region
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPG

SEQ ID NO: 73 - protein, 334 aa, synthetic construct
  REGION 1..334: note = can-IgGB-M234A-L235A-P329G
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQTAKTQPREEQFNGTYRVVSVLPIGHQDWLKGKQFTCKVNNKALGSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPG

SEQ ID NO: 74 - protein, 334 aa, synthetic construct
  REGION 1..334: note = can-IgGB-M234A-L235A-P329A
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQTAKTQPREEQFNGTYRVVSVLPIGHQDWLKGKQFTCKVNNKALASPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPG

SEQ ID NO: 75 - protein, 334 aa, synthetic construct
  REGION 1..334: note = can-IgGB-K228P-M234A-L235A-P329G
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPPCPAPEAAGGPSVFIFPPKPKDTLLIARTPEVTCVVVDLDPEDPEVQISWFVDGKQMQTAKTQPREEQFNGTYRVVSVLPIGHQDWLKGKQFTCKVNNKALGSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPG

SEQ ID NO: 76 - protein, 334 aa, synthetic construct
  REGION 1..334: note = can-IgGB-K228P-M234A-L235A-P329A
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPPCPAPEAAGGPSVFIFPPKPKDTLLIARTPEVTCVVVDLDPEDPEVQISWFVDGKQMQTAKTQPREEQFNGTYRVVSVLPIGHQDWLKGKQFTCKVNNKALASPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPG

SEQ ID NO: 77 - protein, 334 aa, synthetic construct
  REGION 1..334: note = can-IgGB-N297G-P329G
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFGGTYRVVSVLPIGHQDWLKGKQFTCKVNNKALGSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPG

SEQ ID NO: 78 - protein, 334 aa, synthetic construct
  REGION 1..334: note = can-IgGB-N297G-P329A
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFGGTYRVVSVLPIGHQDWLKGKQFTCKVNNKALASPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPG

SEQ ID NO: 79 - protein, 334 aa, synthetic construct
  REGION 1..334: note = can-IgGB-N297A-P329G
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFAGTYRVVSVLPIGHQDWLKGKQFTCKVNNKALGSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPG

SEQ ID NO: 80 - protein, 334 aa, synthetic construct
  REGION 1..334: note = canIgGB-N297A-P329A
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFAGTYRVVSVLPIGHQDWLKGKQFTCKVNNKALASPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPG

SEQ ID NO: 81 - protein, 334 aa, synthetic construct
  REGION 1..334: note = can-IgGB-K228P-N297G
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPPCPAPEMLGGPSVFIFPPKPKDTLLIARTPEVTCVVVDLDPEDPEVQISWFVDGKQMQTAKTQPREEQFGGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPG

SEQ ID NO: 82 - protein, 334 aa, synthetic construct
  REGION 1..334: note = can-IgGB-K228P-N297A
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPPCPAPEMLGGPSVFIFPPKPKDTLLIARTPEVTCVVVDLDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPG

SEQ ID NO: 83 - protein, 334 aa, synthetic construct
  REGION 1..334: note = canIgGB-D265A-N297G
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVALDPEDPEVQISWFVDGKQMQTAKTQPREEQFGGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPG

SEQ ID NO: 84 - protein, 334 aa, synthetic construct
  REGION 1..334: note = canIgGB-D265A-N297A
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPG

SEQ ID NO: 85 - protein, 334 aa, synthetic construct
  REGION 1..334: note = canIgGB-N297G
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFGGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPG

SEQ ID NO: 86 - protein, 334 aa, synthetic construct
  REGION 1..334: note = canIgGB-N297A
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPG

SEQ ID NO: 87 - protein, 106 aa, synthetic construct
  REGION 1..106: note = lambda type constant region of the light chain
GQPKASPSVTLFPPSSEELGANKATLVCLISDFYPSGVTVAWKADGSPVTQGVETTKPSKQSNNKYAASSYLSLTPDKWKSHSSFSCLVTHECSTVEKKVAPAECS

SEQ ID NO: 88 - protein, 106 aa, synthetic construct
  REGION 1..106: note = kappa type constant region of the light chain
RNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSEC

SEQ ID NO: 89 - protein, 515 aa, synthetic construct
  REGION 1..515: note = cECD-IL31RA
VLPAKPENISCIFYYEENFTCTWSPEKEASYTWYKVKRTYSYGYKSDICSTDNSTRGNHASCSFLPPTITNPDNYTIQVEAQNADGIMKSDITYWNLDAIMKIEPPEIFSVKSVLGIKRMLQIKWIRPVLAPHSSTLKYTLRFRTINSAYWMEVNFTKEDIDRDETYNLTELQAFTEYVMTLRCAPAESMFWSGWSQEKVGTTEEEAPYGLDLWRVLKPAMVDGRRPVQLMWKKATGAPVLEKALGYNIWYFPENNTNLTETVNTTNQTHELYLGGKTYWVYVVSYNSLGESPVATLRIPALNEKTFQCIEAMQACLTQDQLVVEWQSSAPEVDTWMVEWFPDVDSEPSSFSWESVSQARNWTIQKDELKPLWCYNISVYPVLRDRVGQPYSTQAYVQEGIPSAGPVTQADSIGVKTVTITWKEIPKSKRNGFIKNYTIFYQAEDGKEFSKTVNSNILQYRLESLTRRTSYSLQVMASTNAGGTNGTKINFKTLSISVLEGGGGSHHHHHHHHHH

SEQ ID NO: 90 - protein, 119 aa, synthetic construct
  REGION 1..119: note = Can 8D3-VH-B
EVTLQESGPGLVKPSQTLSLTCVASGFSIKDSFIHWLRQRPGRGLEWIGRIDPAQGNTEYDPNLQGRISITADTAKNQASLQLSSMTTEDTAVYYCARYYYGNAHFDSWGQGTLVTVSS

SEQ ID NO: 91 - protein, 119 aa, synthetic construct
  REGION 1..119: note = Can 8D3-VH-G
EVTLQESGPGLVKPSQTLSLTCVASGFSIKDSFIHWLRQRPGRGLEWIGRIDPAQGNTEYDPNLQGRISITADTAKNQASLQLSSMTTEDTAVYYCARYYYGASHFDSWGQGTLVTVSS

SEQ ID NO: 92 - DNA, 13 bp, synthetic construct
  misc_feature 1..13: note = Kozak consensus sequence
  source 1..13: mol_type = other DNA
gccgccrccatgg

SEQ ID NO: 93 - protein, 10 aa, synthetic construct
  REGION 1..10: note = Chim CDR-H-3 alternative
YYYGNSHFDA

SEQ ID NO: 94 - protein, 119 aa, synthetic construct
  REGION 1..119: note = VH with Chim CDR-H-3 alternative
EVQLQQSGAELVKPGASVKLSCTASGFNIKDSFIHWLKQRPEQGLEWIGRIDPANGNTEYDPNFQGKVTITADTSSNTAYLQLSSLTSEDTAVYYCARYYYGNSHFDAWGQGTTLTVSS

SEQ ID NO: 95 - protein, 18 aa, synthetic construct
  REGION 1..18: note = GS18 linker
GSTSGGGSGGGSGGGGSS

SEQ ID NO: 96 - protein, 49 aa, synthetic construct
  REGION 1..49: note = GS50 linker
GSSGSGSGSTGTSSSGTGTSAGTTGTSASTSGSGSGGGGGSGGGGSAGG

SEQ ID NO: 97 - protein, 477 aa, synthetic construct
  REGION 1..477: note = Derivate Format 1
EVQLQQSGAELVKPGASVKLSCTASGFNIKDSFIHWLKQRPEQGLEWIGRIDPANGNTEYDPNFQGKVTITADTSSNTAYLQLSSLTSEDTAVYYCARYYYGNSHFDAWGQGTTLTVSSGSTSGGGSGGGSGGGGSSRIVMNQTPKFLPISAGDRVIITCKASQSVTNDVTWYQQKPGQSPKVLIHYASNRYTGVPDRFTGSGYGTDFTFTISTVQAEDLAVYFCQQDYSSPFTFGSGTKLTVLGPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 98 - protein, 477 aa, synthetic construct
  REGION 1..477: note = Derivate Format 2
EVQLQQSGAELVKPGASVKLSCTASGFNIKSSFIHWLKQRPEQGLEWIGRIDPAFGATEYNPAFQGKVTITADTSSNTAYLQLSSLTSEDTAVYYCARYHYAASHFDAWGQGTTLTVSSGSTSGGGSGGGSGGGGSSRIVMNQTPKFLPISAGDRVIITCKSSQSVTNDLTWYQQKPGQSPKVLIHYASQRYTGVPDRFTGSGYGTDFTFTISTVQAEDLAVYFCQQDYASPFTFGSGTKLTVLGPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 99 - protein, 481 aa, synthetic construct
  REGION 1..481: note = Derivate Format 3
EVTLQESGPGLVKPSQTLSLTCVASGFSIKSSFIHWLRQRPGRGLEWIGRIDPAFGATEYNPAFQGRFSITADTAKNQASLQLSSMTTEDSAVYYCARYHYAASHFDAWGQGTLVTVSSGSTSGGGSGGGSGGGGSSRIVMTQSPGSLAGSVGESVSINCKSSQSVTNDLTWYQQKPGEAPKVLITYASQRYTGVPARFSGSGYGTDFTLTINNLQAEDVGDYFCQQDYASPFTFGGGTKLTVLGPKRENGRVPRPPDCPKCPAPEAAGGPSVFIFPPKPKDTLLIARTPEVTCVVVDLDPEDPEVQISWFVDGKQMQTAKTQPREEQFNGTYRVVSVLPIGHQDWLKGKQFTCKVNNKALGSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPG

SEQ ID NO: 100 - protein, 480 aa, synthetic construct
  REGION 1..480: note = Derivate Format 4
EVTLQESGPGLVKPSQTLSLTCVASGFSIKSSFIHWLRQRPGRGLEWIGRIDPAFGATEYNPAFQGRFSITADTAKNQASLQLSSMTTEDSAVYYCARYHYAASHFDAWGQGTLVTVSSGSTSGGGSGGGSGGGGSSRIVMTQSPGSLAGSVGESVSINCKSSQSVTNDLTWYQQKPGEAPKVLITYASQRYTGVPARFSGSGYGTDFTLTINNLQAEDVGDYFCQQDYASPFTFGQGTKLEIKPKRENGRVPRPPDCPKCPAPEAAGGPSVFIFPPKPKDTLLIARTPEVTCVVVDLDPEDPEVQISWFVDGKQMQTAKTQPREEQFNGTYRVVSVLPIGHQDWLKGKQFTCKVNNKALGSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPG

SEQ ID NO: 101 - protein, 716 aa, synthetic construct
  REGION 1..716: note = Derivate Format 5
RIVMTQSPGSLAGSVGESVSINCKSSQSVTNDLTWYQQKPGEAPKVLITYASQRYTGVPARFSGSGYGTDFTLTINNLQAEDVGDYFCQQDYASPFTFGQGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECGGSSGSGSGSTGTSSSGTGTSAGTTGTSASTSGSGSGGGGGSGGGGSAGGEVTLQESGPGLVKPSQTLSLTCVASGFSIKSSFIHWLRQRPGRGLEWIGRIDPAFGATEYNPAFQGRFSITADTAKNQASLQLSSMTTEDSAVYYCARYHYAASHFDAWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEAAGGPSVFIFPPKPKDTLLIARTPEVTCVVVDLDPEDPEVQISWFVDGKQMQTAKTQPREEQFNGTYRVVSVLPIGHQDWLKGKQFTCKVNNKALGSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPG

SEQ ID NO: 102 - protein, 480 aa, synthetic construct
  REGION 1..480: note = Derivate Format 6
RIVMTQSPGSLAGSVGESVSINCKSSQSVTNDLTWYQQKPGEAPKVLITYASQRYTGVPARFSGSGYGTDFTLTINNLQAEDVGDYFCQQDYASPFTFGQGTKLEIKGSTSGGGSGGGSGGGGSSEVTLQESGPGLVKPSQTLSLTCVASGFSIKSSFIHWLRQRPGRGLEWIGRIDPAFGATEYNPAFQGRFSITADTAKNQASLQLSSMTTEDSAVYYCARYHYAASHFDAWGQGTLVTVSSPKRENGRVPRPPDCPKCPAPEAAGGPSVFIFPPKPKDTLLIARTPEVTCVVVDLDPEDPEVQISWFVDGKQMQTAKTQPREEQFNGTYRVVSVLPIGHQDWLKGKQFTCKVNNKALGSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPG

SEQ ID NO: 103 - protein, 481 aa, synthetic construct
  REGION 1..481: note = Derivate Format 7
RIVMTQSPGSLAGSVGESVSINCKSSQSVTNDLTWYQQKPGEAPKVLITYASQRYTGVPARFSGSGYGTDFTLTINNLQAEDVGDYFCQQDYASPFTFGGGTKLTVLGGSTSGGGSGGGSGGGGSSEVTLQESGPGLVKPSQTLSLTCVASGFSIKSSFIHWLRQRPGRGLEWIGRIDPAFGATEYNPAFQGRFSITADTAKNQASLQLSSMTTEDSAVYYCARYHYAASHFDAWGQGTLVTVSSPKRENGRVPRPPDCPKCPAPEAAGGPSVFIFPPKPKDTLLIARTPEVTCVVVDLDPEDPEVQISWFVDGKQMQTAKTQPREEQFNGTYRVVSVLPIGHQDWLKGKQFTCKVNNKALGSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPG

SEQ ID NO: 104 - protein, 11 aa, synthetic construct
  REGION 1..11: note = VL C-terminal region corresponding to mouse lambda J gene
FGSGTKLTVLG

SEQ ID NO: 105 - protein, 11 aa, synthetic construct
  REGION 1..11: note = VL C-terminal region corresponding to canine lambda J gene
FGGGTKLTVLG

SEQ ID NO: 106 - protein, 245 aa, synthetic construct
  REGION 1..245: note = Derivate Format 1 without Fc
EVQLQQSGAELVKPGASVKLSCTASGFNIKDSFIHWLKQRPEQGLEWIGRIDPANGNTEYDPNFQGKVTITADTSSNTAYLQLSSLTSEDTAVYYCARYYYGNSHFDAWGQGTTLTVSSGSTSGGGSGGGSGGGGSSRIVMNQTPKFLPISAGDRVIITCKASQSVTNDVTWYQQKPGQSPKVLIHYASNRYTGVPDRFTGSGYGTDFTFTISTVQAEDLAVYFCQQDYSSPFTFGSGTKLTVLG

SEQ ID NO: 107 - protein, 245 aa, synthetic construct
  REGION 1..245: note = Derivate Format 2 without Fc
EVQLQQSGAELVKPGASVKLSCTASGFNIKSSFIHWLKQRPEQGLEWIGRIDPAFGATEYNPAFQGKVTITADTSSNTAYLQLSSLTSEDTAVYYCARYHYAASHFDAWGQGTTLTVSSGSTSGGGSGGGSGGGGSSRIVMNQTPKFLPISAGDRVIITCKSSQSVTNDLTWYQQKPGQSPKVLIHYASQRYTGVPDRFTGSGYGTDFTFTISTVQAEDLAVYFCQQDYASPFTFGSGTKLTVLG

SEQ ID NO: 108 - protein, 245 aa, synthetic construct
  REGION 1..245: note = Derivate Format 3 without Fc
EVTLQESGPGLVKPSQTLSLTCVASGFSIKSSFIHWLRQRPGRGLEWIGRIDPAFGATEYNPAFQGRFSITADTAKNQASLQLSSMTTEDSAVYYCARYHYAASHFDAWGQGTLVTVSSGSTSGGGSGGGSGGGGSSRIVMTQSPGSLAGSVGESVSINCKSSQSVTNDLTWYQQKPGEAPKVLITYASQRYTGVPARFSGSGYGTDFTLTINNLQAEDVGDYFCQQDYASPFTFGGGTKLTVLG

SEQ ID NO: 109 - protein, 244 aa, synthetic construct
  REGION 1..244: note = Derivate Format 4 without Fc
EVTLQESGPGLVKPSQTLSLTCVASGFSIKSSFIHWLRQRPGRGLEWIGRIDPAFGATEYNPAFQGRFSITADTAKNQASLQLSSMTTEDSAVYYCARYHYAASHFDAWGQGTLVTVSSGSTSGGGSGGGSGGGGSSRIVMTQSPGSLAGSVGESVSINCKSSQSVTNDLTWYQQKPGEAPKVLITYASQRYTGVPARFSGSGYGTDFTLTINNLQAEDVGDYFCQQDYASPFTFGQGTKLEIK

SEQ ID NO: 110 - protein, 480 aa, synthetic construct
  REGION 1..480: note = Derivate Format 5 without Fc
RIVMTQSPGSLAGSVGESVSINCKSSQSVTNDLTWYQQKPGEAPKVLITYASQRYTGVPARFSGSGYGTDFTLTINNLQAEDVGDYFCQQDYASPFTFGQGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECGGSSGSGSGSTGTSSSGTGTSAGTTGTSASTSGSGSGGGGGSGGGGSAGGEVTLQESGPGLVKPSQTLSLTCVASGFSIKSSFIHWLRQRPGRGLEWIGRIDPAFGATEYNPAFQGRFSITADTAKNQASLQLSSMTTEDSAVYYCARYHYAASHFDAWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPV

SEQ ID NO: 111 - protein, 244 aa, synthetic construct
  REGION 1..244: note = Derivate Format 6 without Fc
RIVMTQSPGSLAGSVGESVSINCKSSQSVTNDLTWYQQKPGEAPKVLITYASQRYTGVPARFSGSGYGTDFTLTINNLQAEDVGDYFCQQDYASPFTFGQGTKLEIKGSTSGGGSGGGSGGGGSSEVTLQESGPGLVKPSQTLSLTCVASGFSIKSSFIHWLRQRPGRGLEWIGRIDPAFGATEYNPAFQGRFSITADTAKNQASLQLSSMTTEDSAVYYCARYHYAASHFDAWGQGTLVTVSS

SEQ ID NO: 112 - protein, 245 aa, synthetic construct
  REGION 1..245: note = Derivate Format 7 without Fc
RIVMTQSPGSLAGSVGESVSINCKSSQSVTNDLTWYQQKPGEAPKVLITYASQRYTGVPARFSGSGYGTDFTLTINNLQAEDVGDYFCQQDYASPFTFGGGTKLTVLGGSTSGGGSGGGSGGGGSSEVTLQESGPGLVKPSQTLSLTCVASGFSIKSSFIHWLRQRPGRGLEWIGRIDPAFGATEYNPAFQGRFSITADTAKNQASLQLSSMTTEDSAVYYCARYHYAASHFDAWGQGTLVTVSS

SEQ ID NO: 113 - protein, 108 aa, synthetic construct
  REGION 1..108: note = VL-H canine lambda J gene
RIVMTQSPGSLAGSVGESVSINCKSSQSVTNDLTWYQQKPGEAPKVLITYASQRYTGVPARFSGSGYGTDFTLTINNLQAEDVGDYFCQQDYASPFTFGGGTKLTVLG

SEQ ID NO: 114 - protein, 4 aa, synthetic construct
  REGION 1..4: note = peptide linker for derivatives
GGGS

SEQ ID NO: 115 - protein, 5 aa, synthetic construct
  REGION 1..5: note = peptide linker for derivatives
GGGGS

SEQ ID NO: 116 - protein, 5 aa, synthetic construct
  REGION 1..5: note = peptide linker for derivatives
GSGSG

SEQ ID NO: 117 - protein, 5 aa, synthetic construct
  REGION 1..5: note = peptide linker for derivatives
SGSGS

SEQ ID NO: 118 - protein, 15 aa, synthetic construct
  REGION 1..15: note = peptide linker for derivatives
GGGGSGGGGSGGGGS

SEQ ID NO: 119 - protein, 10 aa, synthetic construct
  REGION 1..10: note = peptide linker for derivatives
GSGSGSGSGS

SEQ ID NO: 120 - protein, 5 aa, synthetic construct
  REGION 1..5: note = peptide linker for derivatives
GSTSG

SEQ ID NO: 121 - protein, 5 aa, synthetic construct
  REGION 1..5: note = peptide linker for derivatives
SGTGS